(19) United States
(12) Patent Application Publication
(10) Pub. No.: US 2015/0157584 A1
(43) Pub. Date: Jun. 11, 2015

(54) INHIBITORS OF HIPPO-YAP SIGNALING PATHWAY

(71) Applicants: The Regents of the University of California, Oakland, CA (US); The J. David Gladstone Institutes, San Francisco, CA (US)

(72) Inventors: Kun-Liang Guan, San Diego, CA (US); Faxing Yu, San Diego, CA (US); Sheng Ding, San Diego, CA (US)

(73) Assignees: The J. David Gladstone Institutes, San Francisco, CA (US); The Regents of the University of California, Oakland, CA (US)

(21) Appl. No.: 14/406,749

(22) PCT Filed: May 31, 2013
(86) PCT No.: PCT/US2013/043752

§ 371 Date: Dec. 9, 2014

Related U.S. Application Data
(60) Provisional application No. 61/658,796, filed on Jun. 12, 2012.
(60) Provisional application No. 61/658,084, filed on Jun. 11, 2012.

(51) Int. Cl. A61K 31/15; A61K 31/44; A61K 31/277; A61K 31/451; A61K 31/437; A61K 39/395; A61K 31/522; A61K 31/661; A61K 38/48; A61K 38/26; A61K 31/138; A61K 31/4453; A61K 31/4015; A61K 31/404

(52) U.S. Cl. CPC A61K 31/15; A61K 31/4015; A61K 31/44; A61K 31/277; A61K 31/451; A61K 31/437; A61K 31/404; A61K 31/522; A61K 31/661; A61K 38/4833; A61K 38/26; A61K 31/138; A61K 31/4453; A61K 39/39558

(57) ABSTRACT

This invention provides methods of preventing, reducing, delaying or inhibiting the proliferation, growth, migration and/or metastasis of cancer by administering an effective amount of an inhibitor of the Hippo-YAP signaling pathway.

### CROSS-REFERENCE TO RELATED APPLICATIONS

This application is a U.S. national phase filing under 35 U.S.C. §371 of Intl. Appl. No. PCT/US2013/043752, filed on May 31, 2013, which claims the benefit under 35 U.S.C. §119(e) of U.S. Provisional Application No. 61/658,084, filed on Jun. 11, 2012, and U.S. Provisional Application No. 61/658,796, filed on Jun. 12, 2012, which are hereby incorporated herein in their entireties for all purposes.

### STATEMENT OF GOVERNMENTAL SUPPORT

This invention was made with government support under Grant No. 5R01CA132809, awarded by the National Institutes of Health. The government has certain rights in the invention.

### FIELD OF THE INVENTION

The present invention relates to preventing, reducing, delaying or inhibiting the proliferation, growth, migration and/or metastasis of cancer by administering an effective amount of an inhibitor of the HIPPO-YAP signaling pathway. In particular, agents that inhibit the activity of TAZ/YAP are provided.

### BACKGROUND OF THE INVENTION

How organ size is controlled in multicellular organisms is a fundamental question in biology. It has been proposed that the mammalian target of rapamycin (mTOR) pathway and the Hippo-YAP pathway control organ size by affecting cell size and cell number, respectively (reviewed in Lee et al., Annu Rev Pharmacol Toxicol (2007) 47:443-467; Zhao et al., Genes Dev (2010) 24:862-874). The Hippo-YAP pathway was initially defined by genetic studies in Drosophila, in which mosaic mutation of the Hippo-YAP pathway genes resulted in tissue overgrowth (reviewed in Pan, Genes Dev (2007) 21:886-897). Function of the Hippo-YAP pathway in organ size regulation is conserved in mammals as demonstrated by studies using genetically modified mouse models (Camargo et al., Curr Biol (2007) 17, 2054-2060; Dong et al., Cell (2007) 130:1120-1133; Heallen et al., Science (2011) 332:458-461; Lee et al., Proc Natl Acad Sci USA (2010) 107:8248-8253; Lu et al., Proc Natl Acad Sci USA (2010) 107:1437-1442; Song et al., Proc Natl Acad Sci USA (2010) 107:1431-1436; Xin et al., Sci Signal (2011) 4:ra70; Zhou et al., Cancer Cell (2009) 16:425-438).

Core components of the mammalian Hippo-YAP pathway include a kinase cascade of Mst1/2 and Lats1/2. Mst1/2, in complex with its regulatory protein salvador (Sav1), phosphorylates and activates Lats1/2 kinases, which also form a complex with its regulatory protein Mob1 (reviewed in Zhao et al., Genes Dev (2010) 24:862-874). The Yes-associated protein (YAP) is a transcription co-activator and a major downstream effector of the Hippo-YAP pathway (Dong et al., Cell (2007) 130:1120-1133). Lats1/2 inhibit YAP by direct phosphorylation, which results in YAP binding to 14-3-3 and cytoplasmic sequestration (Dong et al., 2007, supra; Hao et al., J Biol Chem (2008) 283:5496-5509; Zhao et al., Genes Dev (2007) 21:2747-2761). The unphosphorylated YAP localizes in the nucleus and acts mainly through TEAD family transcription factors to stimulate expression of genes that promote proliferation and inhibit apoptosis (Zhao et al., Genes Dev (2008) 22:1962-1971). Phosphorylation of YAP by Lats1/2 kinases can also promote its ubiquitination-dependent degradation (Zhao et al., Genes Dev (2010) 24:72-85). TAZ is a YAP paralog in mammals and is also regulated by the Hippo-YAP pathway through both cytoplasmic retention and proteasome degradation (Lei et al., Mol Cell Biol (2008) 28:2426-2436).

In transgenic mice, Yap promotes liver enlargement in a reversible manner (Camargo et al., Curr Biol (2007) 17:2054-2060; Dong et al., Cell (2007) 130:1120-1133), suggesting organ size control relies on tight regulation of Hippo-YAP pathway activity. Sustained YAP expression results in hyperplasia or tumor development (Dong et al., 2007, supra), and genetic ablation of Hippo-YAP pathway components in mice also leads to tumor formation (Benhamouche et al., Genes Dev (2010) 24:1718-1730; Cai et al., Genes Dev (2010) 24, 2383-2388; Lee et al., Proc Natl Acad Sci USA (2010) 107:8248-8253; Lu et al., Proc Natl Acad Sci USA (2010) 107:1437-1442; Xu et al., Cancer (2009) 115:4576-4585; Zhang et al., Dev Cell (2010) 19:27-38; Zhou et al., Cancer Cell (2009) 16:425-438). Moreover, abnormal activation of YAP and TAZ has been associated with human cancers (Overholtzer et al., Proc Natl Acad Sci USA (2006) 103:12405-12410; Steinhardt et al., Hum Pathol (2008) 39:1582-1589; Zender et al., Cell (2006) 125:1253-1267; Zhao et al., Genes Dev (2007) 21:2747-2761), suggesting an important role for the Hippo-YAP pathway in tumorigenesis.

Despite extensive studies that have identified many components upstream of the Hippo-YAP pathway such as the NF2 tumor-suppressor (Benhamouche et al., 2010, supra; Hamaratoglu et al., Nat Cell Biol (2006) 8:27-36; Zhang et al., Dev Cell (2010) 19:27-38), no extracellular ligand and cell surface receptor have been identified to modulate the mammalian Hippo-YAP pathway. In this study, we report the identification of various GPCRs and their agonists as Hippo-YAP pathway regulators. Activation of the Gs coupled receptors, such as stimulation by epinephrine or glucagon, increases Lats1/2 kinase activity and thus inhibits YAP function. In contrast, activation of G12/13 or Gq/11 coupled receptors, such as treatment with lysophosphatidic acid (LPA) or sphingosine 1-phosphate (S1P), inhibits Lats1/2 kinases and results in a subsequent YAP activation. Our study demonstrates an important role of the Hippo-YAP pathway in mediating the physiological function of GPCRs and their corresponding extracellular ligands.

The Hippo-YAP signaling pathway was originally discovered in Drosophila, functioning to restrict tissue and organ size. Growth regulation is achieved through inhibition of cell proliferation and promotion of apoptosis (Huang et al. Cell (2005) 122:421-434). Mutation of Hippo-YAP pathway components results in tissue overgrowth and tumor formation (Zhang et al. Cell (2008) 14:377-87; Goulev et al. Curr Biol. (2008) 18:435-41; Neto-Silva et al. Dev Cell (2010) 4:507-520). Components of the Hippo-YAP pathway are highly conserved, and mutation of the Hippo-YAP pathway members, such as NF2, have been associated with human cancer (Zhao et al. Genes Dev (2010) 24:862-874). The core components of the mammalian Hippo-YAP pathway include the Mst kinase and its regulator Sav, Lats kinase and its regulator Mob, as well as the downstream effector Yes associated protein (YAP) and TAZ (Wei et al. EMBO J (2007) 26:1772-81; Saucedo and Edgar, Nat. Rev. Mol. Cell Biol. (2007) 8:613-21). YAP is a transcriptional co-activator that itself has no DNA binding domain but can enhance transcription by binding to specific transcription factors, while TAZ is a YAP paralog in mammals which is also regulated by the Hippo-YAP pathway. The mechanism of Hippo-YAP pathway regulation through its core components has been established recently. Functioning as a kinase cascade, Mst phosphorylates and activates Lats that in turn phosphorylates serine residues in five consensus HXRXXS motifs of YAP inhibiting its transcriptional activation to modulate gene expression (Lin et al. Dev Cell (2011) 21:896-906). Phosphorylation of YAP by Lats has dual inhibitory effects. Lats phosphorylation on S127 of YAP increases 14-3-3 binding and thereby accumulates YAP in the cytoplasm (Ren et al. Dev Biol (2009) 337:303-312). Secondly, phosphorylation of YAP at S381 by Lats promotes YAP ubiquitination and degradation (Zhao et al. Genes Dev (2010) 24:72-85). TAZ is similarly regulated by the Hippo-YAP pathway.

YAP is a known oncogene located in the human amplicon 11q12 (Overholtzer et al. Proc. Natl. Acad. Sci. USA (2006) 103:12405-12410; Zender et al. Cell (2006) 125:1253-1267). Ectopic expression of YAP not only enhances cell growth but also induces oncogenic transformation in vitro. Moreover, YAP can promote epithelial-mesenchymal transition (EMT), a characteristic commonly associated with cancer metastasis. In vivo studies showed that transgenic expression of YAP in mouse liver resulted in a dramatic increase of tissue size/mass, and eventually leads to tumor formation. Similarly, YAP overexpression in intestine and skin results in hyperplasia. YAP gene amplification has been found in human cancers, whereas mutation of the YAP target transcription factor TEAD causes atrophy (Fossdal et al. Hum Mol Genet (2004) 1:975-981), further supporting a role of YAP in tissue growth regulation. YAP activation has been shown in human meduloblastomas, esophageal squamous cell carcinoma, epithelial, ovarian, hepatic and several other types of cancers in recent years (Fernandez et al. Genes Dev (2009) 23:2729-41; Kawano and Inazawa, Carcinogenesis (2011) 32(3):389-398; Zhang et al. Oncogene (2011) 30:2810-2822; Liu et al. Expert Opin Ther Targets. (2010) 8:855-68). In addition, YAP was determined to be an independent prognostic marker for overall survival and disease-free survival for HCC patients (Xu et al. Cancer (2009) 115:19 4576-4585).

While mechanisms governing the upstream regulation of Hippo-YAP pathway are not fully understood, a recent study by our group showed that YAP inactivation after cell detachment induced anoikis, a type of apoptosis that is repressed in cancer cells to promote cell survival and metastasis (Zhao et al. Genes Dev. (2012) 26:54-68). Cell contact signals inhibits YAP, possibly mediated by tight and adheren junctions. In fact, both the tight junction complex protein angiomotin and the adheren junction protein alpha catenin have been shown to directly interact with and inhibit YAP. This mechanism of YAP regulation may be important for the function of YAP in cell contact inhibition. Compelling evidence supports an important physiological role for the Hippo-YAP cascade in normal organ size control and its dysregulation in tumorigenesis (Camargo et al. Curr Biol (2007) 17:2054-2060; Dong et al. Cell (2007) 130:1120-1133). Previously, we and others (Lian et al. Genes Dev (2010) 24:1106-1118; Tamm et al. J. of Cell Sci. (2011) 124:1136-1144) have shown that presence of YAP is required for the maintenance of pluripotency in mouse embryonic stem (ES) cells. Recently, TAZ has been reported to sustain self-renewal and tumor-initiation capacities in breast Cancer Stem Cells (CSCs) (Cordenonsi et al. Cell (2011) 147(4):759-72). Interestingly, several studies have shown that CSCs or cells expressing stem cell markers from multiple cancer types exhibit resistance to conventional cancer therapies (Rich et al. Cell Stem Cell (2007) 1(4):353-5). In line with those observations, elevated YAP expression has also been established as a marker for ovarian cancer and confers resistance to chemotherapeutic agents (Zhang et al. Oncogene (2011) 30:2810-2822). Accumulating evidence suggests tumor growth and recurrence is dependent on CSCs, which represents a specific subset of cellular population in a variety of human tumors. Parallels between ES and cancer cells include shared similarities in transcriptome signatures (Kim et al. Cell (2010) 143(2):313-24), indefinite proliferation, and an undifferentiated state. It is possible that YAP may play critical roles contributing to the phenotypes shared by ES, cancer, and even CSC, making Hippo-YAP pathway an attractive target for oncology study.

High-throughput screenings (HTS) using a reporter assay in mammalian cell culture system is becoming a common approach to identify novel therapeutic candidates in the pharmaceutical industry (Martis et al., Journal of Applied Pharmaceutical Science (2011) 01(01):02-10). Knowledge-based approaches for HTS targeting a specific molecule or pathway have been widely adapted as popular strategy for drug discovery. In this report, we isolated chemical inhibitors based on a YAP dependent transcription assay. The C108 compound potently inhibits YAP by promoting YAP ubiquitination and degradation. Moreover, C108 inhibits cell growth in with high background YAP activity and reduces tumor cell growth in a xenograft mouse model, demonstrating the value of targeting this pathway for cancer therapy.

### SUMMARY OF THE INVENTION

In one aspect, the invention provides methods for preventing, reducing, delaying or inhibiting the proliferation, growth, migration and/or metastasis of a cancer cell or tumor, comprising contacting the cancer cell or tumor with an effective amount of an inhibitor of transcriptional coactivator with PDZ binding motif (TAZ)/Yes-associated protein (YAP) transcription co-activator. In some embodiments, the inhibitor of TAZ/YAP is an adenylyl cyclase (AC) activator and/or a phosphodiesterase (PDE) inhibitor. In some embodiments, the inhibitor of TAZ/YAP comprises a 9H-Fluoren-9-one, oxime pharmacophore of Formula II:

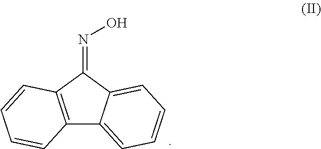 [SMILES: ON=C1c2ccccc2-c2ccccc21]

In another aspect, the invention provides methods of preventing, reducing, delaying or inhibiting the proliferation, growth, migration and/or metastasis of a cancer or tumor mediated, at least in part, by activation of transcriptional coactivator with PDZ binding motif (TAZ)/Yes-associated protein (YAP) transcription co-activator in a subject in need thereof. In some embodiments, the methods comprise administering to the subject an effective amount of an adenylyl cyclase (AC) activator and/or a phosphodiesterase (PDE) inhibitor. In some embodiments, the methods comprise administering to the subject an effective amount of an inhibitor of TAZ/YAP, wherein the inhibitor of TAZ/YAP comprises a 9H-Fluoren-9-one, oxime pharmacophore of Formula II:

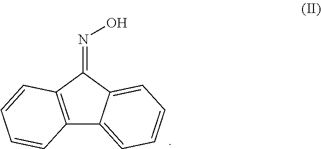 [SMILES: ON=C1c2ccccc2-c2ccccc21]

In a further embodiment, the invention provides methods of preventing, reducing and/or inhibiting the dephosphorylation of transcriptional coactivator with PDZ binding motif (TAZ)/Yes-associated protein (YAP) transcription co-activator and/or promoting and/or increasing the ubiquitination and/or degradation of TAZ/YAP in a cancer cell or tumor, comprising contacting the cancer cell or tumor with an effective amount of an inhibitor of TAZ/YAP. In some embodiments, the inhibitor of TAZ/YAP is an adenylyl cyclase (AC) activator and/or a phosphodiesterase (PDE) inhibitor. In some embodiments, the inhibitor of TAZ/YAP comprises a 9H-Fluoren-9-one, oxime pharmacophore of Formula II:

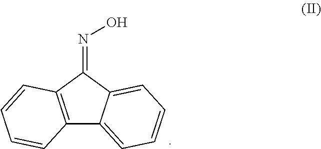 [SMILES: ON=C1c2ccccc2-c2ccccc21]

In various embodiments, the 9H-Fluoren-9-one, oxime pharmacophore is substituted or unsubstituted. In some embodiments, the inhibitor of TAZ/YAP comprises an oxime derivative of 9-fluorenone bearing one or two piperidinylsulfonyl groups. In various embodiments, the one or two piperidinylsulfonyl groups are attached at carbon positions 1, 2, 3, 4, 5, 6, 7 and/or 8 of the 9H-Fluoren-9-one, oxime pharmacophore. In various embodiments, a first piperidinylsulfonyl group is attached at carbon position 1, 2, 3 or 4 of the 9H-Fluoren-9-one, oxime pharmacophore and a second piperidinylsulfonyl group is attached at carbon positions 5, 6, 7 or 8 of the 9H-Fluoren-9-one, oxime pharmacophore. In various embodiments, a first piperidinylsulfonyl group is attached at carbon position 2 of the 9H-Fluoren-9-one, oxime pharmacophore and a second piperidinylsulfonyl group is attached at carbon positions 7 of the 9H-Fluoren-9-one, oxime pharmacophore.

In some embodiments, the inhibitor of TAZ/YAP comprises

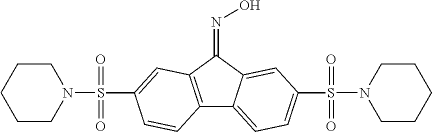 [SMILES: O=S(=O)(c1ccc2c(c1)C(=NO)c1cc(S(=O)(=O)N3CCCCC3)ccc1-2)N1CCCCC1]

### 2,7-bis(piperidin-1-yl-sulfonyl)-9H-fluoren-9-one oxime

In some embodiments, the proliferation and/or growth of the cancer cell is mediated, at least in part, by unphosphorylated TAZ/YAP. In some embodiments, the inhibitor of TAZ/YAP prevents, reduces or inhibits the dephosphorylation and/or nuclear translocation and/or localization of TAZ/YAP. In some embodiments, the inhibitor of TAZ/YAP promotes and/or increases the phosphorylation and/or degradation, e.g., ubiquitination-dependent degradation, of TAZ/YAP.

In some embodiments, the inhibitor of TAZ/YAP is administered orally, intravenously, inhalationally, transdermally, subcutaneously, intratumorally or intramuscularly.

In a further aspect, the invention provides methods of preventing, reducing, delaying or inhibiting the proliferation, growth, migration and/or metastasis of a cancer cell or tumor. In some embodiments, the methods comprise contacting the cancer cell or tumor with an effective amount of an inhibitor of a Gα-protein selected from the group consisting of G12, G13, Gq, G11, Gi and Go or an antagonist of a G-protein-coupled receptor (GPCR) coupled to a G protein selected from the group consisting of G12, G13, Gq, G11, Gi and Go. In another aspect, the invention provides methods of preventing, reducing, delaying or inhibiting the proliferation, growth, migration and/or metastasis of a cancer or tumor mediated, at least in part, by activation of TAZ/YAP in a subject in need thereof. In some embodiments, the methods comprise administering to the subject an effective amount an inhibitor of a Gα-protein selected from the group consisting of G12, G13, Gq, G11, Gi and Go or an inhibitor of a G-protein-coupled receptor (GPCR) coupled to a Gα-protein selected from the group consisting of G12, G13, Gq, G11, Gi and Go. In some embodiments, antagonist binding and signaling through a G protein-coupled receptor (GPCR) selected from the group consisting of lysophosphatidic acid receptor 1-5 (LPAR1-5), sphingosine 1-phosphate receptors, coagulation factor II (thrombin) receptors, estrogen receptor 1 (GPR30), frizzled homolog D4, bombesin-like receptor 3, adrenergic receptor alpha 1B, purinergic receptor 1, purinergic receptor type A, 5-hydroxytryptamine receptor 4, muscarinic acetylcholine receptor M1, adenosine receptor A1A, angiotensin II receptor, free fatty acid receptor 1, platelet-activating factor receptor, thromboxane receptor A2, complement component 3a receptor 1, glutamate receptor metabotropic 2, opioid receptor delta 1, secretin receptor, thyroid stimulating hormone receptor, gastrin-releasing peptide receptor, melanocortin receptor 1, somatostatin receptor 1 and prostaglandin E receptor 2 inhibits TAZ/YAP. In some embodiments, antagonist binding and signaling through a G protein-coupled receptor (GPCR) selected from the group consisting of lysophosphatidic acid receptor 1-5 (LPAR1-5), sphingosine 1-phosphate receptors, coagulation factor II (thrombin) receptors, estrogen receptor 1 (GPR30) and frizzled homolog D4 inhibits TAZ/YAP.

In another aspect, the invention provides methods of preventing, reducing, delaying or inhibiting the proliferation, growth, migration and/or metastasis of a cancer cell or tumor. In some embodiments, the methods comprise contacting the cancer cell or tumor with an effective amount of an activator of a Gs Gα-protein or an agonist of a G-protein-coupled receptor (GPCR) coupled to a Gs Gα-protein. In some embodiments, the invention provides methods of preventing, reducing, delaying or inhibiting the proliferation, growth, migration and/or metastasis of a cancer or tumor mediated, at least in part, by activation of TAZ/YAP in a subject in need thereof. In some embodiments, the methods comprise administering to the subject an effective amount of an activator of a Gs Gα-protein or an agonist of a G-protein-coupled receptor (GPCR) coupled to a Gs Gα-protein. In some embodiments, agonist activation through a G-protein-coupled receptor (GPCR) selected from the group consisting of endothelin receptor type A, chemokine (C—X—C motif) receptor 4, CXCR2, adrenergic receptor beta 2, dopamine receptor D1, glucagon receptor, and epinephrine receptor inhibits TAZ/YAP.

In another aspect, the invention provides methods of preventing, reducing, delaying or inhibiting the proliferation, growth, migration and/or metastasis of a cancer cell or tumor. In some embodiments, the methods comprise contacting the cancer cell or tumor with an effective amount of an activator of adenylyl cyclase (AC) and/or an inhibitor of phosphodiesterase (PDE). In a further aspect, the invention provides methods of preventing, reducing, delaying or inhibiting the proliferation, growth, migration and/or metastasis of a cancer or tumor mediated by activation of TAZ/YAP in a subject in need thereof. In some embodiments, the methods comprise administering to the subject an effective amount of an activator of adenylyl cyclase (AC) and/or an inhibitor of phosphodiesterase (PDE). In some embodiments, the inhibitor of phosphodiesterase is an inhibitor of PDE4. In some embodiments, the inhibitor of PDE4 is selected from the group consisting of rolipram, roflumilast, cilomilast, ariflo, HT0712, ibudilast, mesembrine, pentoxifylline, piclamilast, and combinations thereof. In some embodiments, the inhibitor of phosphodiesterase is an inhibitor of PDE5.

In some embodiments, the cancer is selected from the group consisting of melanoma, uveal melanoma, breast cancer, liver cancer, hepatocellular carcinoma, lung adenocarcinoma, glioma, colon cancer, colorectal cancer, mesothelioma, gastric cancer, medulloblastoma, ovarian cancer, esophageal cancer, esophageal squamous cell carcinoma, sarcoma, Ewing sarcoma, head and neck cancer, prostate cancer, and meningioma. In some embodiments, the cancer is selected from the group consisting of melanoma, uveal melanoma, meningioma, angioma, glioma, hepatocellular carcinoma, breast cancer, ovarian cancer and lung cancer (e.g., lung adenocarcinoma). In some embodiments, the cancer is selected from the group consisting of uveal melanoma, meningioma and breast cancer.

In some embodiments, the cancer cell or tumor is in vivo. In some embodiments, the cancer cell or tumor is in vitro.

In some embodiments, the cancer cell or tumor is in a human subject.

In another aspect, the invention provides methods of preventing, reducing and/or inhibiting signaling through the HIPPO-YAP/TAZ cell signaling pathway and/or preventing, reducing and/or inhibiting YAP/TAZ activation and/or dephosphorylation in a cell, comprising contacting the cell with an antagonist of a G protein-coupled receptor (GPCR) selected from the group consisting of lysophosphatidic acid receptor 1-5 (LPAR1-5), sphingosine 1-phosphate receptors, coagulation factor II (thrombin) receptors, estrogen receptor 1 (GPR30), frizzled homolog D4, bombesin-like receptor 3, adrenergic receptor alpha 1B, purinergic receptor 1, purinergic receptor type A, 5-hydroxytryptamine receptor 4, muscarinic acetylcholine receptor M1, adenosine receptor A1A, angiotensin II receptor, free fatty acid receptor 1, platelet-activating factor receptor, thromboxane receptor A2, complement component 3a receptor 1, glutamate receptor metabotropic 2, opioid receptor delta 1, secretin receptor, thyroid stimulating hormone receptor, gastrin-releasing peptide receptor, melanocortin receptor 1, somatostatin receptor 1 and prostaglandin E receptor 2. In some embodiments, the G protein-coupled receptor (GPCR) selected from the group consisting of lysophosphatidic acid receptor 1-5 (LPAR1-5), sphingosine 1-phosphate receptors, coagulation factor II (thrombin) receptors, estrogen receptor 1 (GPR30) and frizzled homolog D4.

In another aspect, the invention provides methods of preventing, reducing and/or inhibiting signaling through the HIPPO-YAP/TAZ cell signaling pathway and/or preventing, reducing and/or inhibiting YAP/TAZ activation and/or dephosphorylation in a cell, comprising contacting the cell with an agonist of a G protein-coupled receptor (GPCR) selected from the group consisting of endothelin receptor type A, chemokine (C—X—C motif) receptor 4 (CXCR4), CXCR2, adrenergic receptor beta 2, dopamine receptor D1, glucagon receptor, and epinephrine receptor.

In another aspect, the invention provides methods of preventing, reducing and/or inhibiting signaling through the HIPPO-YAP/TAZ cell signaling pathway and/or preventing, reducing and/or inhibiting YAP/TAZ activation and/or dephosphorylation in a cell, comprising contacting the cell with an actin disrupting agent.

In a further aspect, the invention provides methods of preventing, reducing and/or inhibiting signaling through the HIPPO-YAP/TAZ cell signaling pathway and/or preventing, reducing and/or inhibiting YAP/TAZ activation and/or dephosphorylation in a cell. In some embodiments, the methods comprise contacting the cell with an activator of adenylyl cyclase (AC) and/or an inhibitor of phosphodiesterase (PDE). In some embodiments, the inhibitor of phosphodiesterase is an inhibitor of PDE4. In varying embodiments, the inhibitor of PDE4 is selected from the group consisting of rolipram, roflumilast, cilomilast, ariflo, HT0712, ibudilast, mesembrine, pentoxifylline, piclamilast, and combinations thereof. In some embodiments, the inhibitor of phosphodiesterase is an inhibitor of PDE5.

In various embodiments, the cell is a cancer cell.

In another aspect, the invention provides methods of a preventing, reducing and/or inhibiting signaling through the HIPPO-YAP/TAZ cell signaling pathway and/or preventing, reducing and/or inhibiting signaling through the HIPPO-YAP/TAZ cell signaling pathway in a cell, comprising contacting the cell with a ligand selected from the group consisting of glucagon, epinephrine and a dopamine receptor agonist.

In another aspect, the invention provides methods of activating, promoting and/or increasing signaling through the HIPPO-YAP/TAZ cell signaling pathway and/or activating, promoting and/or increasing signaling through the HIPPO-YAP/TAZ cell signaling pathway in a cell, comprising contacting the cell with an agonist of a G protein-coupled receptor (GPCR) selected from the group consisting of lysophosphatidic acid receptor 1-5 (LPAR1-5), sphingosine 1-phosphate receptors, coagulation factor II (thrombin) receptors, estrogen receptor 1 (GPR30), frizzled homolog D4, bombesin-like receptor 3, adrenergic receptor alpha 1B, a lysophosphatidic acid receptor, purinergic receptor 1, purinergic receptor type A, 5-hydroxytryptamine receptor 4, muscarinic acetylcholine receptor M1, adenosine receptor A1A, angiotensin II receptor, free fatty acid receptor 1, platelet-activating factor receptor, thromboxane receptor A2, complement component 3a receptor 1, glutamate receptor metabotropic 2, opioid receptor delta 1, secretin receptor, thyroid stimulating hormone receptor, gastrin-releasing peptide receptor, melanocortin receptor 1, somatostatin receptor 1 and prostaglandin E receptor 2. In some embodiments, the G protein-coupled receptor (GPCR) selected from the group consisting of lysophosphatidic acid receptor 1-5 (LPAR1-5), sphingosine 1-phosphate receptors, coagulation factor II (thrombin) receptors, estrogen receptor 1 (GPR30), frizzled homolog D4, CXCR2 and CXCR4.

In another aspect, the invention provides methods of activating, promoting and/or increasing signaling through the HIPPO-YAP/TAZ cell signaling pathway and/or activating, promoting and/or increasing signaling through the HIPPO-YAP/TAZ cell signaling pathway in a cell, comprising contacting the cell with an antagonist of a G protein-coupled receptor (GPCR) selected from the group consisting of endothelin receptor type A, chemokine (C—X—C motif) receptor 4 (CXCR4), CXCR2, adrenergic receptor beta 2, dopamine receptor D1, glucagon receptor, and epinephrine receptor.

In another aspect, the invention provides methods of activating, promoting and/or increasing signaling through the HIPPO-YAP/TAZ cell signaling pathway and/or activating, promoting and/or increasing YAP/TAZ activation and/or dephosphorylation in a cell, comprising contacting the cell with a ligand selected from the group consisting of lysophosphatidic acid, sphingosine 1-phosphate (S1P) and thrombin.

In another aspect, the invention provides methods of a activating, promoting and/or increasing signaling through the HIPPO-YAP/TAZ cell signaling pathway and/or activating, promoting and/or increasing signaling through the HIPPO-YAP/TAZ cell signaling pathway in a cell, comprising contacting the cell with a ligand selected from the group consisting of glucagon, epinephrine and a dopamine receptor antagonist.

In some embodiments, the cell is in vivo. In some embodiments, the cell is in vitro.

In another aspect, the invention provides methods of reducing or inhibiting the proliferation, growth, invasiveness and/or migration of a cell, comprising contacting the cell with an effective amount of an inhibitor of TAZ/YAP. In some embodiments, the inhibitor of TAZ/YAP is an adenylyl cyclase (AC) activator and/or a phosphodiesterase (PDE) inhibitor. In some embodiments, the inhibitor of TAZ/YAP comprises a 9H-Fluoren-9-one, oxime pharmacophore of Formula II:

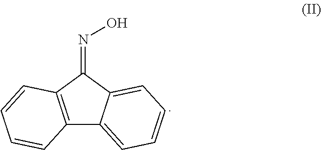 [SMILES: ON=C1c2ccccc2-c2ccccc21]

In another aspect, the invention provides methods of preventing, reducing and/or inhibiting the dephosphorylation of transcriptional coactivator with PDZ binding motif (TAZ)/Yes-associated protein (YAP) transcription co-activator and/or promoting and/or increasing the ubiquitination and/or degradation of TAZ/YAP in a cell, comprising contacting the cell with an effective amount of an inhibitor of TAZ/YAP. In some embodiments, the inhibitor of TAZ/YAP is an adenylyl cyclase (AC) activator and/or a phosphodiesterase (PDE) inhibitor. In some embodiments, the inhibitor of TAZ/YAP comprises a 9H-Fluoren-9-one, oxime pharmacophore of Formula II:

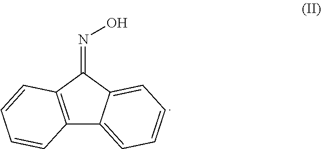 [SMILES: ON=C1c2ccccc2-c2ccccc21]

In various embodiments, the 9H-Fluoren-9-one, oxime pharmacophore is substituted or unsubstituted. In some embodiments, the inhibitor of TAZ/YAP comprises an oxime derivative of 9-fluorenone bearing one or two piperidinylsulfonyl groups. In various embodiments, the one or two piperidinylsulfonyl groups are attached at carbon positions 1, 2, 3, 4, 5, 6, 7 and/or 8 of the 9H-Fluoren-9-one, oxime pharmacophore. In various embodiments, a first piperidinylsulfonyl group is attached at carbon position 1, 2, 3 or 4 of the 9H-Fluoren-9-one, oxime pharmacophore and a second piperidinylsulfonyl group is attached at carbon positions 5, 6, 7 or 8 of the 9H-Fluoren-9-one, oxime pharmacophore. In various embodiments, a first piperidinylsulfonyl group is attached at carbon position 2 of the 9H-Fluoren-9-one, oxime pharmacophore and a second piperidinylsulfonyl group is attached at carbon positions 7 of the 9H-Fluoren-9-one, oxime pharmacophore.

In some embodiments, the inhibitor of TAZ/YAP comprises

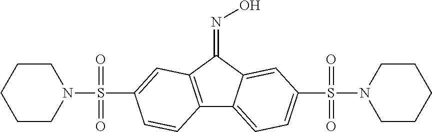 [SMILES: O=S(=O)(c1ccc2c(c1)C(=NO)c1cc(S(=O)(=O)N3CCCCC3)ccc1-2)N1CCCCC1]

### 2,7-bis(piperidin-1-yl-sulfonyl)-9H-fluoren-9-one oxime

In some embodiments, the inhibitor of TAZ/YAP prevents, reduces or inhibits YAP/TAZ protein levels and/or the dephosphorylation and/or nuclear localization of TAZ/YAP.

In some embodiments, the cell is in vivo. In some embodiments, the cell is in vitro. In some embodiments, the cell is in a human subject.

In some embodiments, the inhibitor of TAZ/YAP is administered orally, intravenously, inhalationally, transdermally, subcutaneously or intramuscularly.

### DEFINITIONS

The “Yes-associated protein (YAP) transcription co-activator” refers to nucleic acids and polypeptide polymorphic variants, alleles, mutants, and interspecies homologs that: (1) have an amino acid sequence that has greater than about 90% amino acid sequence identity, for example, 91%, 92%, 93%, 94%, 95%, 96%, 97%, 98% or 99% or greater amino acid sequence identity, preferably over a region of at least about 25, 50, 100, 200, 400, or more amino acids, or over the full-length, to an amino acid sequence encoded by a YAP nucleic acid (see, e.g., GenBank Accession No. NM_{—}001130145.2→NP_{—}001123617.1 yorkie homolog isoform 1; NM_{—}006106.4→NP_{—}006097.2 yorkie homolog isoform 2; NM_{—}001195044.1→NP_{—}001181973.1 yorkie homolog isoform 3; 3.NM_{—}001195045.1→NP_{—}001181974.1 yorkie homolog isoform 4); (2) bind to antibodies, e.g., polyclonal antibodies, raised against an immunogen comprising an amino acid sequence of a YAP polypeptide; or an amino acid sequence encoded by a YAP nucleic acid, and conservatively modified variants thereof; (3) specifically hybridize under stringent hybridization conditions to an anti-sense strand corresponding to a nucleic acid sequence encoding a YAP protein, and conservatively modified variants thereof; (4) have a nucleic acid sequence that has greater than about 90%, preferably greater than about 91%, 92%, 93%, 94%, 95%, 96%, 97%, 98%, 99%, or higher nucleotide sequence identity, preferably over a region of at least about 25, 50, 100, 200, 500, 1000, 2000 or more nucleotides, or over the full-length, to a YAP nucleic acid (e.g., described above). Based on the knowledge of YAP homologs, those of skill can readily determine residue positions that are more tolerant to substitution. For example, amino acid residues conserved amongst species are less tolerant of substitution or deletion. Similarly, amino acid residues that are not conserved amongst species are more tolerant of substitution or deletion, while retaining the function of the YAP protein. YAP is the human ortholog of chicken YAP protein which binds to the SH3 domain of the Yes proto-oncogene product. This protein contains a WW domain that is found in various structural, regulatory and signaling molecules in yeast, nematode, and mammals, and may be involved in protein-protein interaction. As discussed above, functionally, YAP is a transcription co-activator and a major downstream effector of the Hippo-YAP pathway (Dong et al., 2007). Lats1/2 inhibit YAP by direct phosphorylation, which results in YAP binding to 14-3-3 and cytoplasmic sequestration (Dong et al., 2007; Hao et al., 2008; Zhao et al., 2007). The unphosphorylated YAP localizes in the nucleus and acts mainly through TEAD family transcription factors to stimulate expression of genes that promote proliferation and inhibit apoptosis (Zhao et al., 2008). Phosphorylation of YAP by Lats1/2 kinases can also promote its ubiquitination-dependent degradation (Zhao et al., 2010b).

The terms “WW domain containing transcription regulator 1 (WWTR1)” and “transcriptional coactivator with PDZ binding motif (TAZ)” refers to a YAP paralog in mammals and is also regulated by the Hippo-YAP pathway through both cytoplasmic retention and proteasome degradation (Lei et al., 2008). TAZ is a WW domain containing a transcription coactivator that modulates mesenchymal differentiation and development of multiple organs. TAZ refers to nucleic acids and polypeptide polymorphic variants, alleles, mutants, and interspecies homologs that: (1) have an amino acid sequence that has greater than about 90% amino acid sequence identity, for example, 91%, 92%, 93%, 94%, 95%, 96%, 97%, 98% or 99% or greater amino acid sequence identity, preferably over a region of at least about 25, 50, 100, 200, 400, or more amino acids, or over the full-length, to an amino acid sequence encoded by a TAZ nucleic acid (see, e.g., GenBank Accession Nos. NM_{—}001168278.1→NP_{—}001161750.1; 2.NM_{—}001168280.1→NP_{—}001161752.1; NM_{—}015472.4→NP_{—}056287.1; see also, Kanai, et al., The EMBO Journal (2000) 19(24):6778-6791); (2) bind to antibodies, e.g., polyclonal antibodies, raised against an immunogen comprising an amino acid sequence of a TAZ polypeptide; or an amino acid sequence encoded by a TAZ nucleic acid, and conservatively modified variants thereof; (3) specifically hybridize under stringent hybridization conditions to an anti-sense strand corresponding to a nucleic acid sequence encoding a TAZ protein, and conservatively modified variants thereof; (4) have a nucleic acid sequence that has greater than about 90%, preferably greater than about 91%, 92%, 93%, 94%, 95%, 96%, 97%, 98%, 99%, or higher nucleotide sequence identity, preferably over a region of at least about 25, 50, 100, 200, 500, 1000, 2000 or more nucleotides, or over the full-length, to a TAZ nucleic acid (e.g., described above). Based on the knowledge of TAZ homologs, those of skill can readily determine residue positions that are more tolerant to substitution. For example, amino acid residues conserved amongst species are less tolerant of substitution or deletion. Similarly, amino acid residues that are not conserved amongst species are more tolerant of substitution or deletion, while retaining the function of the TAZ protein.

As used herein, “administering” refers to local and systemic administration, e.g., including enteral, parenteral, pulmonary, and topical/transdermal administration. Routes of administration for an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) that find use in the methods described herein include, e.g., oral (per os (P.O.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intratumoral administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intra-arterial, intradermal, subcutaneous, intraperitoneal, intraventricular, ionophoretic and intracranial. Other modes of delivery include, but are not limited to, the use of liposomal formulations, intravenous infusion, transdermal patches, etc.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “co-administering” or “concurrent administration”, when used, for example with respect to the agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) and/or analogs thereof and another active agent (e.g., a cognition enhancer), refers to administration of the compound and/or analogs and the active agent such that both can simultaneously achieve a physiological effect. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the body (e.g., in the plasma) at a significant fraction (e.g., 20% or greater, preferably 30% or 40% or greater, more preferably 50% or 60% or greater, most preferably 70% or 80% or 90% or greater) of their maximum serum concentration for any given dose.

The term “effective amount” or “pharmaceutically effective amount” refer to the amount and/or dosage, and/or dosage regime of one or more compounds necessary to bring about the desired result e.g., an amount sufficient to mitigating in a mammal one or more symptoms associated with cancer (e.g., therapeutically effective amounts), an amount sufficient to reduce the risk or delaying the onset, and/or reduce the ultimate severity of a cancer in a mammal (e.g., prophylactically effective amounts).

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

As used herein, the terms “treating” and “treatment” refer to delaying the onset of, retarding or reversing the progress of, reducing the severity of, or alleviating or preventing either the disease or condition to which the term applies, or one or more symptoms of such disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease. In certain embodiments, the reduction or elimination of one or more symptoms of pathology or disease can include, but is not limited to, reduction or elimination of tumor burden and/or metastasis.

The terms “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child) under the care of a physician or other healthworker in a hospital, psychiatric care facility, as an outpatient, or other clinical context. In certain embodiments the subject may not be under the care or prescription of a physician or other healthworker.

The term “agonist” refers to moieties or agents that interact with (e.g., bind to) and activate a receptor, e.g., a G-protein-coupled receptor, and initiate a physiological or pharmacological response characteristic of that receptor, for example, moieties that activate the intracellular response upon binding to the receptor.

The term “antagonist” refers to moieties that interact with (e.g., bind to) and inhibit a receptor, e.g., a G-protein-coupled receptor, and reduce, prevent or inhibit a physiological or pharmacological response characteristic of that receptor, for example, moieties or agents that reduce, prevent or inhibit the intracellular response upon binding to the receptor.

The term “pharmacophore” refers to molecular features for molecular recognition or binding of a ligand or agent by a biological macromolecule. In various embodiments, “pharmacophore” refers to an ensemble of steric and electronic features for supramolecular interactions with a specific biological target and to trigger or block its biological response (e.g., prevents, reduces and/or inhibits the activity (e.g., the dephosphorylation and/or nuclear translocation and/or localization) of TAZ/YAP).

“Alkyl” in its broadest sense is intended to include linear, branched, or cyclic hydrocarbon structures, and combinations thereof. Alkyl groups can be fully saturated or with one or more units of unsaturation, but not aromatic. Generally alkyl groups are defined by a subscript, either a fixed integer or a range of integers. For example, “C₈alkyl” includes n-octyl, iso-octyl, 3-octynyl, cyclohexenylethyl, cyclohexylethyl, and the like; where the subscript “8” designates that all groups defined by this term have a fixed carbon number of eight. In another example, the term “C₁₋₆alkyl” refers to alkyl groups having from one to six carbon atoms and, depending on any unsaturation, branches and/or rings, the requisite number of hydrogens. Examples of C₁₋₆alkyl groups include methyl, ethyl, vinyl, propyl, isopropyl, butyl, s-butyl, t-butyl, isobutyl, isobutenyl, pentyl, pentynyl, hexyl, cyclohexyl, hexenyl, and the like. When an alkyl residue having a specific number of carbons is named generically, all geometric isomers having that number of carbons are intended to be encompassed. For example, either “propyl” or “C₃alkyl” each include n-propyl, c-propyl, propenyl, propynyl, and isopropyl. Cycloalkyl is a subset of alkyl and includes cyclic hydrocarbon groups of from three to thirteen carbon atoms. Examples of cycloalkyl groups include c-propyl, c-butyl, c-pentyl, norbornyl, norbornenyl, c-hexenyl, adamantyl and the like. As mentioned, alkyl refers to alkanyl, alkenyl, and alkynyl residues (and combinations thereof)—it is intended to include, e.g., cyclohexylmethyl, vinyl, allyl, isoprenyl, and the like. An alkyl with a particular number of carbons can be named using a more specific but still generic geometrical constraint, e.g. “C₃₋₆cycloalkyl” which means only cycloalkyls having between 3 and 6 carbons are meant to be included in that particular definition. Unless specified otherwise, alkyl groups, whether alone or part of another group, e.g. —C(O)alkyl, have from one to twenty carbons, that is C₁₋₂₀alkyl. In the example “—C(O)alkyl,” where there were no carbon count limitations defined, the carbonyl of the —C(O)alkyl group is not included in the carbon count, since “alkyl” is designated generically. But where a specific carbon limitation is given, e.g. in the term “optionally substituted C₁₋₂₀alkyl,” where the optional substitution includes “oxo” the carbon of any carbonyls formed by such “oxo” substitution are included in the carbon count since they were part of the original carbon count limitation. However, again referring to “optionally substituted C₁₋₂₀alkyl,” if optional substitution includes carbon-containing groups, e.g. CH₂CO₂H, the two carbons in this group are not included in the C₁₋₂₀alkyl carbon limitation.

When a carbon number limit is given at the beginning of a term which itself comprises two terms, the carbon number limitation is understood as inclusive for both terms. For example, for the term “C₇₋₁₄arylalkyl,” both the “aryl” and the “alkyl” portions of the term are included the carbon count, a maximum of 14 in this example, but additional substituent groups thereon are not included in the atom count unless they incorporate a carbon from the group's designated carbon count, as in the “oxo” example above. Likewise when an atom number limit is given, for example “6-14 membered heteroarylalkyl,” both the “heteroaryl” and the “alkyl” portion are included the atom count limitation, but additional substituent groups thereon are not included in the atom count unless they incorporate a carbon from the group's designated carbon count. In another example, “C₄₋₁₀cycloalkylalkyl” means a cycloalkyl bonded to the parent structure via an alkylene, alkylidene or alkylidyne; in this example the group is limited to 10 carbons inclusive of the alkylene, alkylidene or alkylidyne subunit. As another example, the “alkyl” portion of, e.g. “C₇₋₁₄arylalkyl” is meant to include alkylene, alkylidene or alkylidyne, unless stated otherwise, e.g. as in the terms “C₇₋₁₄arylalkylene” or “C₆₋₁₀aryl-CH₂CH₂—.”

“Alkylene” refers to straight, branched and cyclic (and combinations thereof) divalent radical consisting solely of carbon and hydrogen atoms, containing no unsaturation and having from one to ten carbon atoms, for example, methylene, ethylene, propylene, n-butylene and the like. Alkylene is like alkyl, referring to the same residues as alkyl, but having two points of attachment and, specifically, fully saturated. Examples of alkylene include ethylene (—CH₂CH₂—), propylene (—CH₂CH₂CH₂—), dimethylpropylene (—CH₂C(CH₃)₂CH₂—), cyclohexan-1,4-diyl and the like.

“Alkylidene” refers to straight, branched and cyclic (and combinations thereof) unsaturated divalent radical consisting solely of carbon and hydrogen atoms, having from two to ten carbon atoms, for example, ethylidene, propylidene, n-butylidene, and the like. Alkylidene is like alkyl, referring to the same residues as alkyl, but having two points of attachment and, specifically, at least one unit of double bond unsaturation. Examples of alkylidene include vinylidene (—CH═CH—), cyclohexylvinylidene (—CH═C(C₆H₁₃)—), cyclohexen-1,4-diyl and the like.

“Alkylidyne” refers to straight, branched and cyclic (and combinations thereof) unsaturated divalent radical consisting solely of carbon and hydrogen atoms having from two to ten carbon atoms, for example, propylid-2-ynyl, n-butylid-1-ynyl, and the like. Alkylidyne is like alkyl, referring to the same residues as alkyl, but having two points of attachment and, specifically, at least one unit of triple bond unsaturation.

Any of the above radicals “alkylene,” “alkylidene” and “alkylidyne,” when optionally substituted, can contain alkyl substitution which itself can contain unsaturation. For example, 2-(2-phenylethynyl-but-3-enyl)-naphthalene (IUPAC name) contains an n-butylid-3-ynyl radical with a vinyl substituent at the 2-position of the radical. Combinations of alkyls and carbon-containing substitutions thereon are limited to thirty carbon atoms.

“Alkoxy” refers to the group —O-alkyl, where alkyl is as defined herein. Alkoxy includes, by way of example, methoxy, ethoxy, n-propoxy, isopropoxy, n-butoxy, t-butoxy, sec-butoxy, n-pentoxy, cyclohexyloxy, cyclohexenyloxy, cyclopropylmethyloxy, and the like.

“Haloalkyloxy” refers to the group —O-alkyl, where alkyl is as defined herein, and further, alkyl is substituted with one or more halogens. By way of example, a “haloC₁₋₃alkyloxy” group includes —OCF₃, —OCF₂H, —OCHF₂, —OCH₂CH₂Br, —OCH₂CH₂CH₂I, —OC(CH₃)2Br, —OCH₂Cl and the like.

“Acyl” refers to the groups —C(O)H, —C(O)alkyl, —C(O)aryl and C(O)heterocyclyl.

“α-Amino Acids” refer to naturally occurring and commercially available α-amino acids and optical isomers thereof. Typical natural and commercially available α-amino acids are glycine, alanine, serine, homoserine, threonine, valine, norvaline, leucine, isoleucine, norleucine, aspartic acid, glutamic acid, lysine, ornithine, histidine, arginine, cysteine, homocysteine, methionine, phenylalanine, homophenylalanine, phenylglycine, ortho-tyrosine, meta-tyrosine, para-tyrosine, tryptophan, glutamine, asparagine, proline and hydroxyproline. A “side chain of an α-amino acid” refers to the radical found on the α-carbon of an α-amino acid as defined above, for example, hydrogen (for glycine), methyl (for alanine), benzyl (for phenylalanine), etc.

“Amino” refers to the group NH₂.

“Amide” refers to the group C(O)NH₂ or —N(H)acyl.

“Aryl” (sometimes referred to as “Ar”) refers to a monovalent aromatic carbocyclic group of, unless specified otherwise, from 6 to 15 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, 9,10-dihydrophenanthrenyl, indanyl, tetralinyl, and fluorenyl and the like), provided that the point of attachment is through an atom of an aromatic portion of the aryl group and the aromatic portion at the point of attachment contains only carbons in the aromatic ring. If any aromatic ring portion contains a heteroatom, the group is a heteroaryl and not an aryl. Aryl groups are monocyclic, bicyclic, tricyclic or tetracyclic.

“Arylene” refers to an aryl that has at least two groups attached thereto. For a more specific example, “phenylene” refers to a divalent phenyl ring radical. A phenylene, thus can have more than two groups attached, but is defined by a minimum of two non-hydrogen groups attached thereto.

“Arylalkyl” refers to a residue in which an aryl moiety is attached to a parent structure via one of an alkylene, alkylidene, or alkylidyne radical. Examples include benzyl, phenethyl, phenylvinyl, phenylallyl and the like. When specified as “optionally substituted,” both the aryl, and the corresponding alkylene, alkylidene, or alkylidyne portion of an arylalkyl group can be optionally substituted. By way of example, “C₇₋₁₁arylalkyl” refers to an arylalkyl limited to a total of eleven carbons, e.g., a phenylethyl, a phenylvinyl, a phenylpentyl and a naphthylmethyl are all examples of a “C₇₋₁₁arylalkyl” group.

“Aryloxy” refers to the group —O-aryl, where aryl is as defined herein, including, by way of example, phenoxy, naphthoxy, and the like.

“Carboxyl,” “carboxy” or “carboxylate” refers to CO₂H or salts thereof.

“Carboxyl ester” or “carboxy ester” or “ester” refers to the group —CO₂alkyl, —CO₂aryl or —CO₂heterocyclyl.

“Carbonate” refers to the group —OCO₂alkyl, —OCO₂aryl or —OCO₂heterocyclyl.

“Carbamate” refers to the group —OC(O)NH₂, —N(H)carboxyl or —N(H)carboxyl ester.

“Cyano” or “nitrile” refers to the group —CN.

“Formyl” refers to the specific acyl group —C(O)H.

“Halo” or “halogen” refers to fluoro, chloro, bromo and iodo.

“Haloalkyl” and “haloaryl” refer generically to alkyl and aryl radicals that are substituted with one or more halogens, respectively. By way of example “dihaloaryl,” “dihaloalkyl,” “trihaloaryl” etc. refer to aryl and alkyl substituted with a plurality of halogens, but not necessarily a plurality of the same halogen; thus 4-chloro-3-fluorophenyl is a dihaloaryl group.

“Heteroalkyl” refers to an alkyl where one or more, but not all, carbons are replaced with a heteroatom. A heteroalkyl group has either linear or branched geometry. By way of example, a “2-6 membered heteroalkyl” is a group that can contain no more than 5 carbon atoms, because at least one of the maximum 6 atoms must be a heteroatom, and the group is linear or branched. Also, for the purposes of this invention, a heteroalkyl group always starts with a carbon atom, that is, although a heteroalkyl may contain one or more heteroatoms, the point of attachment to the parent molecule is not a heteroatom. A 2-6 membered heteroalkyl group includes, for example, —CH₂XCH₃, —CH₂CH₂XCH₃, —CH₂CH₂XCH₂CH₃, C(CH₂)₂XCH₂CH₃ and the like, where X is O, NH, NC₁₋₆alkyl and S(O)₀₋₂, for example.

“Perhalo” as a modifier means that the group so modified has all its available hydrogens replaced with halogens. An example would be “perhaloalkyl.” Perhaloalkyls include —CF₃, —CF₂CF₃, perchloroethyl and the like.

“Hydroxy” or “hydroxyl” refers to the group —OH.

“Heteroatom” refers to O, S, N, or P.

“Heterocyclyl” in the broadest sense includes aromatic and non-aromatic ring systems and more specifically refers to a stable three- to fifteen-membered ring radical that consists of carbon atoms and from one to five heteroatoms. For purposes of this description, the heterocyclyl radical can be a monocyclic, bicyclic or tricyclic ring system, which can include fused or bridged ring systems as well as spirocyclic systems; and the nitrogen, phosphorus, carbon or sulfur atoms in the heterocyclyl radical can be optionally oxidized to various oxidation states. In a specific example, the group —S(O)₀₋₂—, refers to —S— (sulfide), —S(O)— (sulfoxide), and —SO₂— (sulfone) linkages. For convenience, nitrogens, particularly but not exclusively, those defined as annular aromatic nitrogens, are meant to include their corresponding N-oxide form, although not explicitly defined as such in a particular example. Thus, for a compound having, for example, a pyridyl ring; the corresponding pyridyl-N-oxide is meant to be included in the presently disclosed compounds. In addition, annular nitrogen atoms can be optionally quaternized. “Heterocycle” includes heteroaryl and heteroalicyclyl, that is a heterocyclic ring can be partially or fully saturated or aromatic. Thus a term such as “heterocyclylalkyl” includes heteroalicyclylalkyls and heteroarylalkyls. Examples of heterocyclyl radicals include, but are not limited to, azetidinyl, acridinyl, benzodioxolyl, benzodioxanyl, benzofuranyl, carbazoyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, quinolinyl, isoquinolinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, dihydropyridinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzoxazolyl, furyl, diazabicycloheptane, diazapane, diazepine, tetrahydrofuryl, tetrahydropyranyl, thienyl, benzothieliyl, thiamorpholinyl, thiamorpholinyl sulfoxide, thiamorpholinyl sulfone, dioxaphospholanyl, and oxadiazolyl.

“Heteroaryl” refers to an aromatic group having from 1 to 10 annular carbon atoms and 1 to 4 annular heteroatoms. Heteroaryl groups have at least one aromatic ring component, but heteroaryls can be fully unsaturated or partially unsaturated. If any aromatic ring in the group has a heteroatom, then the group is a heteroaryl, even, for example, if other aromatic rings in the group have no heteroatoms. For example, 2H-pyrido[3,2-b][1,4]oxazin-3(4H)-one-7-yl, indolyl and benzimidazolyl are “heteroaryls.” Heteroaryl groups can have a single ring (e.g., pyridinyl, imidazolyl or furyl) or multiple condensed rings (e.g., indolizinyl, quinolinyl, benzimidazolyl or benzothienyl), where the condensed rings may or may not be aromatic and/or contain a heteroatom, provided that the point of attachment to the parent molecule is through an atom of the aromatic portion of the heteroaryl group. In one embodiment, the nitrogen and/or sulfur ring atom(s) of the heteroaryl group are optionally oxidized to provide for the N-oxide (N→O), sulfinyl, or sulfonyl moieties. Compounds described herein containing phosphorous, in a heterocyclic ring or not, include the oxidized forms of phosphorous. Heteroaryl groups are monocyclic, bicyclic, tricyclic or tetracyclic.

“Heteroaryloxy” refers to O-heteroaryl.

“Heteroarylene” generically refers to any heteroaryl that has at least two groups attached thereto. For a more specific example, “pyridylene” refers to a divalent pyridyl ring radical. A pyridylene, thus can have more than two groups attached, but is defined by a minimum of two non-hydrogen groups attached thereto.

“Heteroalicyclic” refers specifically to a non-aromatic heterocyclyl radical. A heteroalicyclic may contain unsaturation, but is not aromatic. As mentioned, aryls and heteroaryls are attached to the parent structure via an aromatic ring. So, e.g., 2H-1,4-benzoxazin-3(4H)-one-4-yl is a heteroalicyclic, while 2H-1,4-benzoxazin-3(4H)-one-7-yl is an aryl. In another example, 2H-pyrido[3,2-b][1,4]oxazin-3(4H)-one-4-yl is a heteroalicyclic, while 2H-pyrido[3,2-b][1,4]oxazin-3(4H)-one-7-yl is a heteroaryl.

“Heterocyclylalkyl” refers to a heterocyclyl group linked to the parent structure via e.g an alkylene linker, for example (tetrahydrofuran-3-yl)methyl- or (pyridin-4-yl)methyl

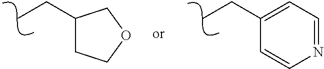

“Heterocyclyloxy” refers to the group —O-heterocycyl.

“Nitro” refers to the group —NO₂.

“Oxo” refers to a double bond oxygen radical, ═O.

“Oxy” refers to —O. radical (also designated as →O), that is, a single bond oxygen radical. By way of example, N-oxides are nitrogens bearing an oxy radical.

When a group with its bonding structure is denoted as being bonded to two partners; that is, a divalent radical, for example, —OCH₂—, then it is understood that either of the two partners can be bound to the particular group at one end, and the other partner is necessarily bound to the other end of the divalent group, unless stated explicitly otherwise. Stated another way, divalent radicals are not to be construed as limited to the depicted orientation, for example “—OCH2-” is meant to mean not only “—OCH₂—” as drawn, but also “—CH₂O—.”

When a group with its bonding structure is denoted as being bonded to two partners; that is, a divalent radical, for example, —OCH₂—, then it is understood that either of the two partners can be bound to the particular group at one end, and the other partner is necessarily bound to the other end of the divalent group, unless stated explicitly otherwise. Stated another way, divalent radicals are not to be construed as limited to the depicted orientation, for example “—OCH₂-” is meant to mean not only “—OCH₂-” as drawn, but also “—CH₂O—.”

“Optional” or “optionally” means that the subsequently described event or circumstance may or may not occur, and that the description includes instances where said event or circumstance occurs and instances in which it does not. One of ordinary skill in the art would understand that, with respect to any molecule described as containing one or more optional substituents, that only synthetically feasible compounds are meant to be included. “Optionally substituted” refers to all subsequent modifiers in a term, for example in the term “optionally substituted arylC₁₋₈alkyl,” optional substitution may occur on both the “C₁₋₈alkyl” portion and the “aryl” portion of the arylC₁₋₈alkyl group. Also by way of example, optionally substituted alkyl includes optionally substituted cycloalkyl groups. The term “substituted,” when used to modify a specified group or radical, means that one or more hydrogen atoms of the specified group or radical are each, independently of one another, replaced with the same or different substituent groups as defined below. Thus, when a group is defined as “optionally substituted” the definition is meant to encompass when the groups is substituted with one or more of the radicals defined below, and when it is not so substituted.

Substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —Se, —N(R⁸⁰)₂, perhaloalkyl, —CN, —OCN, —SCN, —NO, —NO₂, ═N₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —P(O)(O⁻)₂(M⁺)₂, —P(O)(O⁻)₂M²⁺, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)N(R⁸⁰)₂, —C(NR⁷⁰)(R⁸⁰)₂, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)N(R⁸⁰)₂, —NR⁷⁰C(NR⁷⁰)R⁷⁰ and —NR⁷⁰C(NR⁷⁰)N(R⁸⁰)₂, where R⁶⁰ is C₁₋₆alkyl, 3 to 10-membered heterocyclyl, 3 to 10-membered heterocyclylC₁₋₆alkyl, C₆₋₁₀ aryl or C₆₋₁₀ arylC₁₋₆alkyl; each R⁷⁰ is independently for each occurrence hydrogen or R⁶⁰; each R⁸⁰ is independently for each occurrence R⁷⁰ or alternatively, two R⁸⁰'s, taken together with the nitrogen atom to which they are bonded, form a 3 to 7-membered heteroalicyclyl which optionally includes from 1 to 4 of the same or different additional heteroatoms selected from O, N and S, of which N optionally has H or C₁-C₃alkyl substitution; and each M⁺ is a counter ion with a net single positive charge. Each M⁺ is independently for each occurrence, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (a “subscript 0.5” means e.g. that one of the counter ions for such divalent alkali earth ions can be an ionized form of a compound described herein and the other a typical counter ion such as chloride, or two ionized compounds can serve as counter ions for such divalent alkali earth ions, or a doubly ionized compound can serve as the counter ion for such divalent alkali earth ions). As specific examples, —N(R⁸⁰)₂ is meant to include —NH₂, —NH-alkyl, —NH-pyrrolidin-3-yl, N-pyrrolidinyl, N-piperazinyl, 4N-methyl-piperazin-1-yl, N-morpholinyl and the like.

Substituent groups for replacing hydrogens on unsaturated carbon atoms in groups containing unsaturated carbons are, unless otherwise specified, —R⁶⁰, halo, —O⁻M⁺, —OR⁷⁰, —SR⁷⁰, —S⁻M⁺, —N(R⁸⁰)₂, perhaloalkyl, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —PO₃⁻²M²⁺, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)N(R⁸⁰)₂, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)N(R⁸⁰)₂, —NR⁷⁰C(NR⁷⁰)R⁷⁰ and —NR⁷⁰C(NR⁷⁰)N(R⁸⁰)₂, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

Substituent groups for replacing hydrogens on nitrogen atoms in groups containing such nitrogen atoms are, unless otherwise specified, —R⁶⁰, —O⁻M⁺, —OR⁷⁰, —SR⁷⁰, —S⁻M⁺, —N(R⁸⁰)₂, perhaloalkyl, —CN, —NO, —NO₂, —S(O)₂R⁷⁰, —SO₃⁻M⁺, —SO₃R⁷⁰, —OS(O)₂R⁷⁰, —OSO₃⁻M⁺, —OSO₃R⁷⁰, —PO₃²⁻(M⁺)₂, —PO₃²⁻M²⁺, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —CO₂R⁷⁰, —C(S)OR⁷⁰, —C(O)NR⁸⁰R⁸⁰, —C(NR⁷⁰)NR⁸⁰R⁸⁰, OC(O)R⁷⁰, —OC(S)R⁷⁰, —OCO₂R⁷⁰, —OC(S)OR⁷⁰, —NR⁷⁰C(O)R⁷⁰, —NR⁷⁰C(S)R⁷⁰, —NR⁷⁰CO₂R⁷⁰, —NR⁷⁰C(S)OR⁷⁰, —NR⁷⁰C(O)N(R⁸⁰)₂, —NR⁷⁰C(NR⁷⁰)R⁷⁰ and —NR⁷⁰C(NR⁷⁰)N(R⁸⁰)₂, where R⁶⁰, R⁷⁰, R⁸⁰ and M⁺ are as previously defined.

In one embodiment, a group that is substituted has 1, 2, 3, or 4 substituents, 1, 2, or 3 substituents, 1 or 2 substituents, or 1 substituent.

It is understood that in all substituted groups, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such case that the language permits such multiple substitutions, the maximum number of such iterations of substitution is three.

“Sulfonamide” refers to the group —SO₂NH₂, —N(H)SO₂H, —N(H)SO₂alkyl, —N(H)SO₂aryl, or —N(H)SO₂heterocyclyl.

“Sulfonyl” refers to the group —SO₂H, —SO₂alkyl, —SO₂aryl, or —SO₂heterocyclyl.

“Sulfanyl” refers to the group: —SH, —S-alkyl, —S-aryl, or —S-heterocyclyl.

“Sulfinyl” refers to the group: —S(O)H, —S(O)alkyl, —S(O)aryl or —S(O)heterocyclyl.

“Suitable leaving group” is defined as the term would be understood by one of ordinary skill in the art; that is, a group on a carbon, where upon reaction a new bond is to be formed, the carbon loses the group upon formation of the new bond. A typical example employing a suitable leaving group is a nucleophilic substitution reaction, e.g., on a sp³ hybridized carbon (SN₂ or SN₁), e.g. where the leaving group is a halide, such as a bromide, the reactant might be benzyl bromide. Another typical example of such a reaction is a nucleophilic aromatic substitution reaction (SNAr). Another example is an insertion reaction (for example by a transition metal) into the bond between an aromatic reaction partner bearing a leaving group followed by reductive coupling. “Suitable leaving group” is not limited to such mechanistic restrictions. Examples of suitable leaving groups include halogens, optionally substituted aryl or alkyl sulfonates, phosphonates, azides and —S(O)₀₋₂R where R is, for example optionally substituted alkyl, optionally substituted aryl, or optionally substituted heteroaryl. Those of skill in the art of organic synthesis will readily identify suitable leaving groups to perform a desired reaction under different reaction.

“Stereoisomer” and “stereoisomers” refer to compounds that have the same atomic connectivity but different atomic arrangement in space. Stereoisomers include cis-trans isomers, E and Z isomers, enantiomers and diastereomers. Compounds described herein, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers can be prepared using chiral synthons, chiral reagents, or resolved using conventional techniques, such as by: formation of diastereoisomeric salts or complexes which can be separated, for example, by crystallization; via formation of diastereoisomeric derivatives which can be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer can be synthesized by asymmetric synthesis using optically active reagents, substrates, catalysts or solvents, or by converting on enantiomer to the other by asymmetric transformation. For a mixture of enantiomers, enriched in a particular enantiomer, the major component enantiomer can be further enriched (with concomitant loss in yield) by recrystallization.

When the compounds described herein contain olefinic double bonds or other centers of geometric asymmetry, and unless specified otherwise, it is intended that the compounds include both E and Z geometric isomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible and contemplated herein.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. Pharmaceutically acceptable acid addition salts are those salts that retain the biological effectiveness of the free bases while formed by acid partners that are not biologically or otherwise undesirable, e.g., inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Pharmaceutically acceptable base addition salts include those derived from inorganic bases such as sodium, potassium, lithium, ammonium, calcium, magnesium, iron, zinc, copper, manganese, aluminum salts and the like. Illustrative salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Illustrative organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.).

“Prodrug” refers to compounds that are transformed in vivo to yield the parent compound, for example, by hydrolysis in the gut or enzymatic conversion in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) where the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention can be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Metabolite” refers to the break-down or end product of a compound or its salt produced by metabolism or biotransformation in the animal or human body; for example, biotransformation to a more polar molecule such as by oxidation, reduction, or hydrolysis, or to a conjugate (see Goodman and Gilman, “The Pharmacological Basis of Therapeutics” 8^{th} Ed., Pergamon Press, Gilman et al. (eds), 1990 which is herein incorporated by reference). The metabolite of a compound described herein or its salt can itself be a biologically active compound in the body. While a prodrug described herein would meet this criteria, that is, form a described biologically active parent compound in vivo, “metabolite” is meant to encompass those compounds not contemplated to have lost a progroup, but rather all other compounds that are formed in vivo upon administration of a compound described herein which retain the biological activities described herein. Thus one aspect of the invention is a metabolite of a compound described herein. For example, a biologically active metabolite is discovered serendipitously, that is, no prodrug design per se was undertaken. Stated another way, biologically active compounds inherently formed as a result of practicing methods of the invention, are contemplated and disclosed herein. “Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. The compounds described herein can exist in unsolvated as well as solvated forms with solvents, pharmaceutically acceptable or not, such as water, ethanol, and the like. Solvated forms of the presently disclosed compounds are contemplated herein and are encompassed by the invention, at least in generic terms.

It is understood that the above definitions are not intended to include impermissible substitution patterns (e.g., methyl substituted with 5 fluoro groups). Such impermissible substitution patterns are easily recognized by a person having ordinary skill in the art.

### BRIEF DESCRIPTION OF THE DRAWINGS

FIGS. 1A-D illustrate serum induces dephosphorylation of YAP and TAZ. (A, B) Serum induces YAP and TAZ dephosphorylation. HEK293A cells were starved in serum free medium for 12 h and then stimulated with 10% FBS for indicated time (A) or with different concentrations of FBS for 1 h (B). Cell lysates were subjected to immunoblotting with indicated antibodies. Gels containing phos-tag was employed in curtain occasion for better assessment of YAP phosphorylation status (A, the bottom panel). l.e. denotes long exposure of Western blots. (C) Serum reversibly affects YAP/TAZ phosphorylation. HEK293A cells were serum-starved and treated with 10% FBS for 1 or 2 h as indicated. In the last three lanes, after 1 h stimulation FBS was removed for ¼, ½ or 1 h as indicated by upwards arrows. (D) Serum induces YAP nuclear localization in HEK293A and MCF10A cells. YAP subcellular localization was determined by immunofluorescence staining for endogenous YAP (green) along with DAPI for DNA (blue). Serum stimulation (10% FBS, 1 h) is indicated. The data presented in this figure and all the subsequent figures are representative of at least three independent experiments.

FIGS. 2A-H illustrate effect of FBS, growth factors, and kinase inhibitors on YAP/TAZ phosphorylation. Cell lysates prepared from different cells lines were used for immunoblotting to assess phosphorylation of YAP/TAZ and other proteins. Unless indicated, serum starved cells were stimulated with 10% FBS or for 1 h. (A) HeLa cells. (B) HeLa cells (phos-tag). (C) RC3, SK-Mel-28 and SF268 cells. (D) U2OS and MCF10A cells (lower panel, phos-tag). (E) and (F), HEK293A cells were treated with EGF (50 ng/ml), PDGF (50 ng/ml), insulin (200 nM), IGF (50, 250 ng/ml) or FGF (10 ng/ml). (G) and (H), HEK293A cells were pre-treated with different inhibitors (1 μM Torin1, 10 μM U0126, 10 μM SB253580, 10 nM Wartmannin (Wart)) for 30 min, and then one group of cells was stimulated with 10% FBS for 1 h as indicated.

FIGS. 3A-F illustrates characterization of serum factor(s) responsible for YAP/TAZ dephosphorylation. (A) Serum contains YAP activating activity. HEK293A cells were treated with 10% of different brands of serum: FBS (from Omega Scientific or Hyclone (HC)), fetal calf serum (FCS), horse serum (HS), or 10% mTesr1. Total cell lysates were subjected to immunoblotting. (B) The YAP-activating activity in serum is protease-resistant. HEK293A cells were treated with FBS that were pre-treated with pronase E or heat inactivated pronase E (HI). The effectiveness of pronase E was demonstrated by Coomassie Blue staining (left panel). Cells were stimulated with control or pronase E treated FBS. (C) YAP activating activity in BSA. Different BSA preparations (from Sigma Aldrich) were used to treat HEK293A cells. A3294 was prepared by heat shock, A7073 Fraction V (FV) and A6003 (fatty acid (FA)-free) were prepared by ethanol precipitation, and A2058 was prepared by chromatography. Protein contents of different BSA preparations were similar as indicated by Coomassie Blue staining (not shown). Serum-starved HEK293A cells were treated with 1 or 10 mg/ml BSA for 1 h before harvest. (D) Charcoal treatment depletes the YAP-activating activity in serum. 10% or 1% of regular or charcoal stripped (Ch) FBS were used to stimulate serum-starved HEK293A cells for 1 h. (E) The YAP-activating activity in FBS is sensitive to organic extraction under acidic conditions. FBS was extracted using chloroform, methanol, or different ratio of chloroform and methanol mixture (CM, in the presence of HCl or NaOH); organic solvent was evaporated and materials extracted were dissolved in 2 mg/ml fatty acid-free BSA (FAF) and used to treat cells. (F) LPA induces YAP dephosphorylation. HEK293A cells were treated with 100 μM of various lipids. Lipids used are phosphatidylserine (PS), phosphatidylcholine (PC), diacylglycerol (DAG), sphingomyelin (SPH), phosphatidylinositol (PI), cardiolipin (CL), phosphatidylethanolamine (PE), phosphatidic acid (PA), phosphatidylglycerol (PG), phosphatidylinositol (3,4)-bisphosphate (PI3,4P) and phosphatidylinositol 3-phosphate (PI3P).

FIGS. 4A-D illustrate LPA and S1P induce YAP/TAZ dephosphorylation. (A) Dose-dependent effect of LPA on YAP/TAZ dephosphorylation. Cells were treated with different concentrations of LPA for 1 h. (B) YAP dephosphorylation induced by different LPA with varying acyl groups (length and saturation). (C) and (D) cells were treated with different doses of LPA, S1P, LPC or PA for 1 h. Cell lysates were subjected to immunoblotting with indicated antibodies. LPA and S1P potently induced YAP dephosphorylation whereas much higher concentrations of PA were needed to induce YAP dephosphorylation. LPC had no effect on YAP phosphorylation. HEK293A cells were used in all experiments.

FIGS. 5A-D illustrate LPA and S1P activate YAP/TAZ by dephosphorylation. HEK293A cells were treated with 1 μM LPA (A) or S1P (B) for indicated times. Cell lysates were subjected to immunoblotting with indicated antibody. (C) Serum and LPA stimulate YAP interaction with TEAD1 but inhibit YAP interaction with 14-3-3. Cells were treated with LPA or serum as indicated. Cell lysates were subjected to immunoprecipitation (IP) with control IgG or YAP antibody. The co-precipitated TEAD1 and 14-3-3 were detected by immunoblotting. (D) LPA treatment (1 μM, for 1 h) induces YAP nuclear localization in HEK293A and MCF10A cells.

FIGS. 6A-B illustrate LPA induces YAP activity. (A) Phosphorylation of YAP at S381 and S384 is inhibited by LPA treatment. HEK293A cells transfected with GFP-YAP was untreated or treated with LPA for 1 h, then phosphorylation of GFP-YAP at S127 and S381/384 was assessed by immunoblotting. (B) LPA induces YAP nuclear localization in a reversible manner. MCF10A cells was serum-starved for 16 h, and then treated with LPA for 1 h. Cells were washed with serum-free medium once and incubated in serum-free medium for indicated time. YAP subcellular localization and actin cytoskeleton was determined by immunofluoresence.

FIGS. 7A-G illustrate YAP/TAZ mediate LPA's cellular functions. (A, B) Over-expression of ATX and LPA1 induce expression of YAP/TAZ targeting genes. HEK293A cells were infected with lentivirus produced using control pLVX-puro vector or Flag-ATX and Flag-LPAR1 expression plasmids. Infected cells were selected with puromycin. Expression of YAP target gene mRNA (panel A) and protein levels of CTGF and Cyr61 (panel B) are shown. (C) Knockdown of YAP/TAZ by shRNA in HEK293A cells. (D) Knockdown of YAP/TAZ by siRNA in MCF10A cells. (E) YAP/TAZ is important for LPA-induced cell migration. Confluent monolayer HEK293A cells (expressing control shRNA or YAP/TAZ shRNA) were serum starved for 24 h after which a uniform wound was made by scratch using a yellow pipette tip. Floating cells were washed away and cells were then incubated in serum-free medium with or without 1 μM LPA. The migration of cells around the wound was assessed under microscope after 12 or 24 h. (F) LPA receptor transgenic expression promotes TAZ nuclear localization. Mammary glands from control (WT) and LPA1 or LPA2 transgenic mice were isolated and fixed. Tissue sections were stained using a TAZ antibody. The cell nuclei were visualized by DAPI staining Regions highlighted by rectangles were enlarged and shown in FIG. 4E. (G) YAP/TAZ and CTGF accumulation in LPA2 tumors. Protein lysates were prepared from mammary glands from three control mice or five tumors from LPA2 transgenic mice, and protein levels of YAP, TAZ and CTGF were assessed by immunoblotting.

FIGS. 8A-F illustrate YAP/TAZ is required for LPA function and is regulated by LPA signaling. (A) YAP/TAZ is required for LPA to induce gene expression. mRNA levels of indicated genes were measured by quantitative PCR. LPA (1 μM) treatment was for 1 h. HEK293A cells with stable knockdown of YAP/TAZ or control cells were used. (B) Knockdown of YAP/TAZ blocks LPA-induced cell migration. Migration of MCF10A cells transfected with control siRNA or YAP/TAZ siRNA was assessed by transwell cell migration assays. (C) YAP/TAZ is required for LPA to stimulate cell proliferation. Control and YAP/TAZ knockdown HEK293A cells were cultured in the absence of FBS and treated with or without 10 μM LPA for 0, 1, 2 or 3 day as indicated, LPA was replenished every day. Cell numbers were then counted. (D) Hyperplasia caused by transgenic LPA1 and LPA2 expression. H & E staining of mammary tissue of wild type and LPA receptor transgenic mice. (E) LPA receptor transgenic expression induces TAZ nuclear localization. Immunofluorescence staining for TAZ (red) and DNA (blue). (F) LPA receptor transgenic expression decreases YAP/TAZ phosphorylation. Sample in each lane was from an individual mouse.

FIGS. 9A-C illustrate the effect of serum and LPA on Hippo kinase activity. (A) Mst kinase activity is not affected by LPA or serum. Endogenous Mst1 protein was immunoprecipitated from FBS (1%) or LPA (1 μM) treated HEK293A cells and in vitro kinase activity was measured using GST-Mob as a substrate. The bottom panel shows YAP phosphorylation in the cell lysates. (B) MST2 phosphorylation is not modulated by LPA. HEK293A cells were transfected with Flag-MST2, after serum-starved for 16 h, cells were untreated or treated with LPA (0.2 or 1 μM) for 1 h. The phosphorylation of MST and YAP were assessed by immunoblotting. (C) Serum and LPA inhibits Lats1 kinase activity. HEK293A cells were treated with FBS or LPA with various durations and concentrations as indicated. Endogenous Lats1 protein was immunoprecipitated and in vitro kinase activity was measured using purified GST-YAP as a substrate and detected by phospho-YAP antibody (top panel). Phosphorylation of endogenous YAP and TAZ in cell lysates was assessed by immunoblotting.

FIGS. 10A-D illustrate LPA and S1P repress Lats kinase activity. (A) MST1/2 are not required for LPA induced YAP dephosphorylation and CTGF induction in MEF cells. WT or knockout MEF cells at similar density were untreated or treated with 1 μM LPA for 1 h, YAP phosphorylation was assessed by immunoblotting in the presence of phos-tag. CTGF expression was also determined. (B) Lats kinase activity is inhibited by LPA. Endogenous Lats1 was immunoprecipitated from HEK293A cells that had been treated with LPA for various time and doses of LPA, and Lats1 kinase activity was determined using GTS-YAP as a substrate. (C) Lats phosphorylation is repressed by LPA. Cell lysates from control or LPA treated (1 μM for 1 h) cells were divided into two parts, one for IgG IP and the other for Lats1 IP. Endogenous Lats1 was immunoprecipitated and probed with phospho-specific antibodies. (D) Lats overexpression suppresses the effect of LPA on YAP phosphorylation. HEK293A cells were co-transfected with Flag-YAP and HA-Lats2 or HA-Mob. 24 h after transfection, cells were serum starved for 24 h, and then treated with 1 μM LPA for 1 h. Cell lysates were prepared for immunoblotting.

FIGS. 11A-I illustrate LPA receptor, G12/13 and Rho GTPase mediate LPA induced YAP activity. (A) Expression of LPA receptors in HEK293A and MCF10A cells. The mRNA level of LPAR1-5 was determined by real-time PCR. (B) Knockdown of LPA1 and LPA3 in HEK293A cells suppresses LPA induced YAP dephosphorylation. Stable cells infected with lentivirus containing expression control shRNA or shRNAs targeting LPA1 and LPA3 were established with puromycin selection. Cells were then treated with 0.04 or 0.2 μM LPA for 5 min, and YAP phosphorylation was determined by immunoblotting. When co-transfected with LPA receptors, the shRNA was able to down-regulate ectopic LPA receptors (upper panel). (C) LPA and S1P receptor expression promote YAP dephosphorylation. Cells were transfected with HA-tagged LPA1-4, S1P1 or S1P2. After 16 h serum-starvation, YAP and TAZ phosphorylation were assessed by immunoblotting. The expression of LPA or S1P receptors was demonstrated by immunoblotting using HA antibody. Protein glycosylation might contribute to heterogenous migration of receptors on SDS-PAGE. (D) The effect of Gα or RhoA overexpression on YAP phosphorylation. HEK293A cells were cotransfected with FLAG-YAP and plasmids expressing constitutively active Gα or Rho (WT or mutant), cells were then incubated in serum free medium (−) or medium with 10% serum (+) for 24 h. YAP phosphorylation were assessed by differential migration on phos-tag containing gels. (E) G12/13 and Rho inhibit Lats kinase activity. HA-Lats2 was co-transfected together with vector, active G12QL, G13QL, and RhoA-L63 in HEK293A cells as indicated. HA-Lats2 was immunoprecipitated and kinase activity was measured using GST-YAP as a substrate. (F) The LPA receptor antagonist Ki16425 blocks the effect of LPA on Lats kinase inhibition. Endogenous Lats1 was immunoprecipitated from cells that had been treated with LPA in the presence or absence of Ki16425 (10 μM for 30 min) and Lats1 kinase activity was determined using GTS-YAP as a substrate. (G) Rho is required for Lats inhibition by LPA, S1P, and serum. HEK293A cells were pre-treated with or without C3 (2 μg/ml C3 for 4 h) before stimulation with LPA, S1P, or serum as indicated. Endogenous Lats1 was immunoprecipitated and kinase activity was measured using GST-YAP as a substrate. (H) LPA induces YAP nuclear localization and stress fibers formation in MCF10A cells. (I) Disruption of actin cytoskeleton blocks the S1P induced YAP nuclear localization. MCF10A cells were pretreated with latrunculin B (Lat B) before stimulation with S1P as indicated.

FIG. 12A-E illustrate LPA and S1P modulate YAP/TAZ through their membrane receptors and Rho GTPases. (A) LPA1/3 antagonist Ki16425 completely blocks LPA and partially blocks serum effect on YAP/TAZ phosphorylation. HEK293 cells were treated with Ki16425 (10 μM) or DMSO control for 30 min as indicated, then cells were stimulated with S1P, LPA or FBS for 1 h. (B). LPA and S1P receptor expression promote YAP nuclear localization. Cells were transfected with HA-tagged LPA1, LPA4, or S1P2 as indicated. The transfected receptors were detected by HA antibody (red) and endogenous YAP were detected by YAP antibody (green). Note the receptor expressing red cells have higher nuclear YAP. (C) Knockdown of G12 and G13 blocks the effect of LPA on YAP phosphorylation. HEK293A cells were transfected with control siRNA, a pool of siRNAs for G12 and G13, or a pool of siRNAs for Gq and G11, serum was removed at 48 h. Following 16 h serum starvation, cells were treated with 1 μM LPA for 1 h. (D) Inactivation of Rho by C3 toxin prevents YAP/TAZ dephosphorylation by LPA, S1P, and serum. HEK293A cells were pretreated with 2 μg/ml C3 for 4 h, then stimulated with LPA, S1P or FBS for 1 h. (E), Disruption of actin cytoskeleton prevents YAP/TAZ dephosphorylation by LPA or serum. HEK293A cells were pretreated with 1 μg/ml LatB for 30 min, then stimulated with LPA or serum for 1 h.

FIGS. 13A-G illustrate stimulation of Gs coupled GPCRs increases YAP phosphorylation. (A) Epinephrine stimulates YAP phosphorylation. MDA-MB-231 cells were treated with indicated concentrations of epinephrine for 1 h. Phosphorylation of CREB was determined by immunoblotting with phospho-CREB specific antibody (pCREB). B) Phosphorylation of YAP from heart of mice injected with epinephrine is increased. Samples from three representative pairs (from strong to weak induction of YAP phosphorylation) of mice were shown. Epinephrine is known to increase blood glucose levels, which are indicated underneath each sample. (C) Dopamine agonist dihydrexidine stimulates YAP phosphorylation. U2OS cells were treated with 10 μM dihydrexidine for 1 h. YAP phosphorylation status was assessed. (D) Glucagon stimulates YAP phosphorylation. Primary mouse hepatocytes were treated with 2 μM glucagon for 1 h, and YAP phosphorylation status was determined. (E) Forskolin induces YAP phosphorylation. MDA-MB-231 cells were treated with different concentrations of Forskolin for 1 h. (F) Forskolin induces Lats1 phosphorylation. Endogenous Lats1 was immunoprecipitated from control cells and Forskolin (Fsk) treated (10 μM for 1 h) HEK293A cells, and protein lysates were divided into two parts, one for IgG IP and the other for Lats1 IP. Proteins precipitated were probed with phospho-specific antibodies against S909 and T1079 of Lats1. (G) A proposed model for GPCRs and G-proteins in regulation of Lats and YAP/TAZ activity.

FIGS. 14A-F illustrate Gs signaling stimulates Lats kinase activity and YAP phosphorylation. (A) Forskolin (Fsk) induces YAP phosphorylation at S127 and S381/384. HEK293A cells transfected with GFP-YAP were treated with or without Forskolin for 1 h. pYAP S381/384 antibody recognizes the S381 and S384 doubly phosphorylated YAP. (B) PKA signaling induces YAP phosphorylation. HEK293A cells were treated with a PKA selective (6-Bnz-cAMP) or Epac selective (8-CPT-2′-Me-cAMP) activators for 1 h. (C) Epinephrine (Epi) and Forskorlin reduced YAP nuclear localization. MDA-MB-231 cells were treated with epinephrine or forskolin for 1 h, and cells were fixed and YAP localization was determined by immunofluoresence staining (D) Epinephrine and LPA antagonize each other on YAP phosphorylation. U2OS cells were serum-starved for 16 h, and cells were then treated with LPA, epinephrine or both for 1 h. (E) Forskolin does not increase MST2 phosphorylation. HEK293A cells transfected with FLAG-MST2 were treated with Forskolin (2 or 10 μM) for 1 h, protein phosphorylation was determined by phospho-specific antibodies. (F) Epinephrine induces Lats1 kinase activity. MDA-MB-231 cells were treated with epinephrine for 15 or 60 min. Endogenous Lats1 were immunoprecipitated and subjected to kinase assay using GST-YAP as substrate, and YAP phosphorylation was assessed by phospho-specific antibody.

FIG. 15 illustrates reporter and effector construct of the cell based luciferase assay. A luminescence assay system consists of UAS Luciferase reporter and Gal4-fused TEAD transcription factor. This reporter activity is strongly stimulated by YAP, which binds to and activates TEAD in transcription.

FIGS. 16A-I illustrate (A) Normalized Luciferase signals corresponding to YAP reporter activity treated by different concentrations of inhibitory small molecules. (B) Structure of Ser. No. 10/590,108 (C108). Western blots of transformed cells (C) BOCS, (D) HEK293A. as well as cancer cell lines (E) glioblastoma SF268, (F) Melanoma M14, (G) Melanoma SK-MEL-28 treated by increasing concentration of C108. (H) Dosage response curves of YAP levels in SF268, M14, and SK-MEL-28 cancer cell lines treated by C108. (I) Western blots of multi-cell line in temporal response to 1 μM of C108.

FIGS. 17A-E illustrate (A) mRNA level of YAP normalized to GAPDH determined by qPCR. (B) Western blot of endogenous YAP level in response to various dosage of C108 w/wo MG132 treatment (C) Endogenous YAP protein level in response to C108 w/wo cycloheximide treatment. (D) Endogenous YAP and (E) total ubiquitin proteins after immunoprecipitated by YAP in respond to C108 treatment.

FIG. 18 illustrates YAP expression in selected cell lines. Western blot of YAP, pYAP127, LAT1, TAZ and Tubulin of selected cell lines.

FIGS. 19A-D illustrate (A) cell migration rate (normalized distance vs. time) of M14 melanoma and (B) Time lapse images of M14 melanoma treated with C108 based on 24 hr scratch assay (C) Migration assay using 22 μM transwell were performed in M14 cells treated with 1, 2 and 3 μM of C108. Quantification of migrated cell population is plotted in (D).

FIGS. 20A-B illustrate that YAP inhibitor C108 retards migration of (A) SK-Mel-28 and (B) EKVX cancer cell lines. Images and migration rate of SK-Mel-28 (upper panels) and EKVX cancer cells (lower panels).

FIG. 21 illustrates Growth curve of M14 Melanoma. Growth curves of M14 Melanoma subjected to dosages of C108.

FIGS. 22A-G illustrate the average of (A) tumor weight and (C) body weight after completion of M14 tumor xenograft study. M14 tumor sizes are plotted (B) during 21 days of C108 treatment. (D) Western blot analysis of YAP and PARP in vehicle control and C108 treated tumor samples. Representation of tumors (E), preserved in Bouin's solution and (F) mice, as well as H & E staining of tumors (G) are shown.

FIGS. 23A-C illustrate that YAP inhibitor C108 retards xenograft tumor growth. (A) Western blot of YAP and Tubulin of tumor tissues (B) Progression of xenograft tumor size (C) Averaged tumor and body weight of EKVX xenograft from control and C108 treated mice.

FIG. 24 illustrates the cAMP signaling and pharmacological interventions used in this study. Stimulation of Gas-coupled receptors by epinephrine, glucagon or other ligands leads to activation of adenylyl cyclase (AC), which results in an increase of cAMP synthesis. The levels of cAMP are also controlled by phosphodiesterases (PDE). Epac and PKA are two major effectors of cAMP. Binding of cAMP to Epac results in activation of Epac and its downstream effector Rap proteins. Under basal conditions, regulatory (R) subunits, C subunits are released from the complex, resulting in PKA activation. Pharmacological inhibitors or activators of AC, PDE or PKA were shown in blue boxes.

FIGS. 25A-F illustrate cAMP signaling induces YAP phosphorylation and inactivation. (A, B) MDA-MB-231 cells were treated with 10 μM of epinephrine (A) or forskolin (B) for indicated durations, and cell lysates were subjected to immunoblotting using indicated antibodies. (C) Time course of YAP and CREB phosphorylation in response to epinephrine or forskolin (value for time zero was arbitrarily set). (D) MDA-MB-231 cells were treated with different PDE inhibitors, ibudilast (100 μM), IBMX (100 μM), rolipram (50 μM) or theophylline (1 mM) for 1 hr, and phosphorylation status of YAP was determined by phos-tag gels. (E) HEK293A, MCF10A, U2OS, or MEF cells were treated with or without 10 μM of forskolin for 1 hr, YAP phosphorylation was assessed using phos-tag gels; the same lysates were also used to blot for TAZ protein levels. (F) MCF10A cells were serum starved overnight and treated with 10 μM of forskolin for 1 or 4 hr, mRNA was extracted and the expression level of CTGF was determined using real-time RT-PCR.

FIGS. 26A-F illustrate cAMP signaling to YAP phosphorylation is mediated by PKA. (A) Flag-YAP was co-transfected with or without HA-tagged wild type or kinase dead PKA catalytic subunit, after 24 hr, cell lysates were prepared and phosphorylation of Flag-YAP was determined. (B) Similar to (A) except Flag-tagged wild type or constitutively active Rap1b was transfected. (C) HEK293A cells were transfected with mutant PKA regulatory subunits (PKARIα or PKARIIα), and after 16 hr cells were treated with or without 10 μM of forskolin for 1 hr, and YAP or CREB phosphorylation was assessed. (D) Stable cell lines (MDA-MB-231) expressing control shRNA or shRNAs targeting PKA catalytic subunit (a isoform) were established, and treated with or without 10 μM of epinephrine or forskolin for 1 hr. Cell lysates were subjected for immunoblotting to determine the level of YAP and CREB phosphorylation. (E) MDA-MB-231 cells were pretreated with or without PKA inhibitor KT5720 (5 μM) for 30 min, then stimulated with 10 μM of epinephrine or forskolin for 1 hr, YAP and CREB phosphorylation were then determined. (F) Similar to (E) except primary hepatocytes were used, and glucagon was used to induce PKA activity.

FIGS. 27A-E illustrate that PKA increases YAP phosphorylation by stimulating kinase activity of Lats1/2. (A) Myc-tagged wild type, S127A or 5SA mutant YAP were transfected into HEK293A cells, and after 16 hr cells were treated with or without 10 μM of forskolin for 1 hr, YAP phosphorylation was assessed by phos-tag gel. (B) MDA-MB-231 cells were transfected with control, MST1/2 or Lats1/2 siRNAs. Two days later, cells were treated with 10 μM of epinephrine or forskolin for 1 hr. Cell lysates were subjected to immunoblotting to assess knockdown efficiency and YAP phosphorylation. The arrowhead indicates MST2 position. (C, D) Flag-YAP was co-transfected into HEK293A cells with or without K/R mutants (kinase dead) of MST2 (C) or Lats2 (D), after 16 hr cells were stimulated with 10 μM of forskolin for 1 hr, phos-tag gels were used to determine phosphorylation status of Flag-YAP. (E) MDA-MB-231 cells were untreated or treated with 10 μM of forskolin for 1 hr, endogenous Lats1 was immunoprecipitated and subjected to kinase assay using GST-YAP as substrate, phosphorylation of GST-YAP by Lats1 was monitored by YAP phosphorylation at S127.

FIGS. 28A-C illustrate Rho GTPases mediate the effect of PKA on YAP phosphorylation. (A) MDA-MB-231 cells were treated with 10 μM of forskolin for 1 hr and cell lysates were subjected to immunoblotting. Phosphorylation of MLC2, CREB, and YAP were determined. (B) Flag-YAP was co-transfected into HEK293A cells with wild type or constitutively active RhoA, and after 16 hrs cells were stimulated with 10 μM of forskolin for 1 hr before Western blotting. (C) Flag-YAP was co-transfected into HEK293A cells with or without GFP-tagged RhoGDI, after 16 hr of incubation in serum free medium, cells were treated with or without KT5720 for 1 hr. Phosphorylation of Flag-YAP and endogenous TAZ was determined.

FIGS. 29A-G illustrate that YAP/TAZ mediate the effect of cAMP in adipogenesis. (A) 3T3-L1 cells were treated with 10 μM of forskolin or 100 μM of IBMX for 1 hr, or serum starved 3T3-L1 cells were treated with 5 μM KT5720 for 1 hr, and YAP phosphorylation and TAZ protein levels were determined. (B) 3T3-L1 cells were incubated under adipocyte differentiation conditions (Tro, troglitazone) with IBMX or KT5720. IBMX increased whereas KT5720 repressed adipogenesis as assessed by oil red staining (C-D) 3T3-L1 cells were transfected with control or YAP and TAZ siRNAs (siYT), and the knockdown efficiency was determined by immunoblotting (C), these cells are also subjected to adipogenesis (D). (E) 3T3-L1 cells were transfected with control or YAP and TAZ siRNAs. Cells were treated Tro and IBMX in the presence of vehicle (DMSO) or KT5720 as indicated. Adipocyte differentiation was measured by oil red staining (F) Overexpression of YAP abolished IBMX or forskolin induced adipogenesis. (G) Following differentiation (as in F), cells were lysed and the expression of adipogenesis marker genes was determined by real-time RT-PCR, the mRNA level was normalized to that of cells incubated in growth medium.

FIGS. 30A-G illustrate that PKA inhibits Yki in Drosophila. (A) In Drosophila S2R+ cells, knockdown of PKA-C1 by RNAi increased Yki/Sd reporter activity. (B) Relative transcript levels of ex, CycE and Diap1 genes in wild-type (blue), C5-Gal4/UAS-PKA-C1 RNAi (red), and C5-Gal4/UAS-PKA-C1 (green) larval wing discs. (C) Yki phosphorylation was increased in C5-Gal4/UAS-PKA-C1 larval wing discs. (D-G) show en-Gal4/UAS-PKA-C1 UAS-GFP larval wing discs exhibiting expression of GFP marker (D, green), Diap1 protein (E, red), Caspase3 (F, white), and merge of D-F (G).

FIG. 31 illustrates regulation of the Hippo-YAP pathway by cAMP-PKA signaling. Upon stimulation of Gas-coupled GPCR, activation of PKA by cAMP leads to inhibition of Rho GTPases, which indirectly inhibit Lats kinase activity. Stimulation of Gα12/13- or Gαq/11-coupled receptors antagonists the effect of cAMP or PKA on YAP phosphorylation by inducing Rho GTPases Inhibition of YAP and TAZ mediates functions of cAMP and PKA on adipogenesis, cell proliferation and apoptosis.

### DETAILED DESCRIPTION

### 1. Introduction

The Hippo-YAP pathway is important in organ size control and its dysregulation contributes to tumorigenesis. Prior to the present invention, upstream signals that regulate the mammalian Hippo-YAP pathway have not been identified. The present invention is based, in part, on the discovery that the Hippo-YAP pathway is regulated by G-protein coupled receptor (GPCR) signaling. Serum-borne lysophosphatidic acid (LPA) and sphingosine 1-phosphate (S1P) act through G12/13 coupled GPCRs to inhibit the Hippo-YAP pathway kinases Lats1/2, thereby activating YAP and TAZ transcription co-activators, which are oncoproteins repressed by Lats1/2. YAP is involved for LPA-induced gene expression, cell migration, and proliferation. In contrast, stimulation of Gs coupled receptors by glucagon or epinephrine activates Lats1/2 kinase activity via PKA, therefore inhibits YAP function. Thus, GPCR signaling can either activate or inhibit the Hippo-YAP pathway in a manner depending on the coupled G-proteins. Our study identifies the first extracellular diffusible signals that modulate the Hippo-YAP pathway activity and also establishes the Hippo-YAP pathway as a critical signaling branch downstream of GPCR.

The present invention is further based, in part, on the discovery of inhibitors of YAP dependent transcription, in particular, a potent inhibitor of YAP, compound C108. Mechanistically, C108 promotes YAP degradation by increasing ubiquitinylation. In addition, C108 inhibits cell proliferation in vitro and reduces growth of xenografted tumors in mice. The present invention identifies the first Hippo-YAP pathway inhibitor, and demonstrates a potential therapeutic value of targeting this pathway for cancer treatment. The YAP transcription co-activator is a downstream target of the Hippo tumor suppressor pathway and has been shown as an important oncogenic factor for multiple types of tumors. Elevated YAP activity increases organ size by stimulating cell proliferation and inhibiting apoptosis. YAP binds to the TEAD family transcription factor to induce gene expression.

### 2. Patients Who May Benefit from Prevention and/or Treatment

Administration of an agent that inhibits the activity of TAZ/YAP (e.g., a direct inhibitor of TAZ/YAP, an activator of PKA (e.g., an adenylyl cyclase (AC) activator and/or a phosphodiesterase (PDE) inhibitor), an inhibitor of a G-protein selected from the group consisting of G12, G13, Gq, G11, Gi and Go or an antagonist of a G-protein-coupled receptor (GPCR) coupled to a G protein selected from the group consisting of G12, G13, Gq, G11, Gi and Go, an activator of a Gs G-protein or an agonist of a G-protein-coupled receptor (GPCR) coupled to a Gs G protein, and mixtures thereof) finds use in preventing, reducing, delaying or inhibiting the proliferation, growth, migration and/or metastasis of a cancer cell or tumor. An agent that directly or indirectly prevents or inhibits the dephosphorylation of TAZ/YAP and/or promotes the phosphorylation and/or degradation of TAZ/YAP can be administered to a patient to effect the inhibition, reduction, retraction or prevention of proliferation, growth, migration and/or metastasis of a tumor or a cancer cell. In the context of effecting treatment, the patient has a cancer or a tumor burden, and administration of an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) can reverse, delay or inhibit progression of the disease. In the context of effecting prevention, the patient may be in remission, or may have undergone the removal of a primary tumor, and administration of an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) can reduce, inhibit or eliminate growth of metastasis. The subject may or may not already be undergoing a regime of a chemotherapeutic agent.

Illustrative cancers mediated by activated and/or unphosphorylated YAP/TAZ that can be treated or prevented by contacting with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) include without limitation melanoma, uveal melanoma (Van Raamsdonk, et al., N Engl J Med (2010) 363, 2191-2199), breast cancer (Yuan, et al., Cell Death Differ (2008) 15(11):1752-9; Tufail et al., Breast Cancer Res Treat. (2012) 131(3):743-50), liver cancer (Liu et al., Expert Opin Ther Targets. (2010) 14(8):855-68; Liu et al., Biochem Biophys Res Commun. (2010) 394(3):623-7; Liu et al., Expert Opin Ther Targets. (2012) 16(3):243-7), hepatocellular carcinoma (Xu et al., Oncogene. (2011) 30(10):1229-40), colon cancer (Avruch et al., Cell Cycle. (2012) 11(6):1090-6; Zhou et al., Proc Natl Acad Sci USA. (2011) 108(49):E1312-20), colorectal carcinoma (Konsavage et al., J Biol Chem. (2012) 287(15):11730-9), mesothelioma (Mizuno et al., Oncogene. (2012) Jan. 30. PMID:22286761), gastric cancer (Zhou et al., Hepatogastroenterology. (2011) 58(112):2156-61; Zhou et al., Mol Med Report. (2011) 4(6):1075-82), medulloblastoma (Fernandez, et al., Oncogene. (2012) 31(15):1923-37), ovarian cancer (Zhang et al., Oncogene. (2011) 30(25):2810-22; Hall et al., Cancer Res. (2010) 70(21):8517-25), esophageal cancer, esophageal squamous cell carcinoma (Muramatsu et al., Carcinogenesis. (2011) 32(3):389-98), sarcoma, Ewing sarcoma (Hsu et al., Oncogene. (2011) 30(17):2077-85), head and neck cancer (Ehsanian et al., Oncogene. (2010) 29(46):6160-71), prostate cancer (Zagurovskaya et al., Oncogene. (2009) 28(8):1121-31; Zhao et al., Genes Dev. (2007) 21(21):2747-61), and meningioma (Striedinger, et al., Neoplasia (2008) 10(11):1204-12).

Illustrative cancers that can be treated or prevented by contacting with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) include without limitation lymphoma, lung cancer, breast cancer, ovarian cancer, gastric and intestinal cancers (including colon cancer and rectal cancer), hepatic cancer, esophageal cancer, bladder cancer, renal cancer, head and neck cancers. In some embodiments, the cancer produces solid tumors. In some embodiments, the cancer is an epithelial cancer or a carcinoma, a sarcoma, or a hematological cancer.

Illustrative hematologic malignancies that can be treated or prevented by contacting with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) include without limitation lymphomas (such as but not limited to, non-Hodgkin's lymphoma, including Burkitt's lymphoma, and Hodgkin's lymphoma, as well as all subtypes associated with each), acute lymphocytic leukemia (ALL), chronic lymphocytic leukemia (CLL), acute myeloid leukemia (AML), chronic myeloid leukemia (CML), and adult T-cell leukemia lymphoma.

Illustrative lung cancers that can be treated or prevented by contacting with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) include without limitation adenocarcinoma, squamous carcinoma, bronchial carcinoma, broncoalveloar carcinoma, large cell carcinoma, small-cell carcinoma, non-small cell lung carcinoma and metastatic lung cancer refractory to conventional chemotherapy.

Illustrative hematological cancers that can be treated or prevented by contacting with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) include without limitation leukemia, multiple myeloma and plasmocytoma.

Illustrative sarcomas that can be treated or prevented by contacting with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) include without limitation rhabdomyosarcoma, osteosarcoma, chondrosarcoma, myosarcoma, liposarcoma, fibrosarcoma and Ewing's sarcoma.

Illustrative gastric, digestive and intestinal cancers that can be treated or prevented by contacting with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) include without limitation intestinal carcinoma, rectal carcinoma, colon carcinoma, familial adenomatous polyposis carcinoma, hereditary non-polyposis colorectal cancer, gastric carcinoma, craniopharyngioma, gall bladder carcinoma, esophageal carcinoma, pancreatic carcinoma and adenocarcinoma (including adenocarcinomas of the esophagus and stomach).

Illustrative cancers of the head and neck that can be treated or prevented by contacting with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) include without limitation larynx carcinoma, hypopharynx carcinoma, tongue carcinoma and salivary gland carcinoma.

Illustrative urogenital cancers that can be treated or prevented by contacting with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) include without limitation labial carcinoma, ovarian carcinoma, cervix carcinoma, uterine corpus carcinoma, endometrium carcinoma, chorion carcinoma, prostate carcinoma, testis carcinoma, seminoma, urinary carcinoma, kidney carcinoma, renal carcinoma, and adenocarcinoma (including adenocarcinomas of the vagina, cervix, prostate, and urachus).

Illustrative nervous and sensory system cancers that can be treated or prevented by contacting with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) include without limitation neuroblastoma, brain tumors, meningioma, ependymoma, medulloblastoma, peripheral neuroectodermal tumors, glioblastoma, astrocytoma, oligodendroglioma and retinoblastoma.

Illustrative endocrine and glandular tissue cancers that can be treated or prevented by contacting with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) include without limitation pancreatic carcinoma, medullary thyroid carcinoma, follicular thyroid carcinoma, anaplastic thyroid carcinoma, papillary thyroid carcinoma, pheochromocytoma, adrenal tumors and adenocarcinoma.

Illustrative hepatic cancers that can be treated or prevented by contacting with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) include without limitation hepatocellular carcinoma.

Illustrative skin cancers that can be treated or prevented by contacting with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) include without limitation melanoma, basal cell carcinoma, squamous cell carcinoma and choroids melanoma.

Additional cancers that can be treated or prevented by contacting with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) include without limitation teratomas.

In other embodiments, the subject has a disease or disorder mediated by overactivation of the Hippo-YAP signaling pathway, e.g., a cancer, an inflammatory disorder, a neuronal disorder. In other embodiments, the subject has a disease or disorder mediated by overactivation of LPA and/or S1P signaling, e.g., overactivation of cell signaling through receptors that bind LPA and/or SIP as ligands.

### 3. Modulators of Hippo-YAP Pathway

In various embodiments, the agent that inhibits the Hippo-YAP signaling pathway directly prevents, reduces and/or inhibits the activity (e.g., the phosphorylation and/or nuclear translocation and/or localization) of TAZ/YAP.

a. Oximes

In various embodiments, the inhibitor of TAZ/YAP comprises a compound having a structure of Formula I:

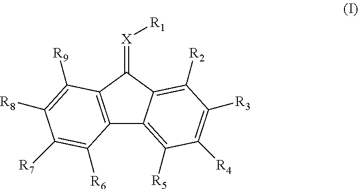

Wherein X is a heteroatom (e.g., O, S, N), and R1, R2, R3, R4, R5, R6, R7, R8, R9 each independently can be H, OH, alkyl, alkylene, alkylidene, alkylidyne, alkoxy, haloalkoxy, acyl, amino, amide, aryl, arylene, arylalkyl, aryloxy, carboxyl, carboxyl ester, carbonate, carbamate, cyano, formyl, halo, haloalkyl, haloaryl, heteroalkyl, perhalo, hydroxyl, heteroatom, heterocyclyl, heteroaryl, heteroaryloxy, heteroarylene, heteroalicyclic, heterocyclylalkyl, heterocyclyloxy, nitro, or oxy. In some embodiments, X is N, R1 is —OH, and one or two of R2, R3, R4, R5, R6, R7, R8, R9 independently comprise a piperidinylsulfonyl group. In some embodiments, X is N, R1 is —OH, and one R2, R3, R4, R5 comprise a piperidinylsulfonyl group and one of R6, R7, R8, R9 comprise a piperidinylsulfonyl group. In some embodiments, X is N, R1 is —OH, and R3 and R8 each comprise a piperidinylsulfonyl group.

In some embodiments, the inhibitor of TAZ/YAP comprises a 9H-Fluoren-9-one, oxime pharmacophore of Formula II:

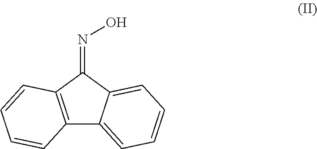 [SMILES: ON=C1c2ccccc2-c2ccccc21]

In various embodiments, the 9H-Fluoren-9-one, oxime can be substituted or unsubstituted, as described above.

In various embodiments, the inhibitor of TAZ/YAP comprises

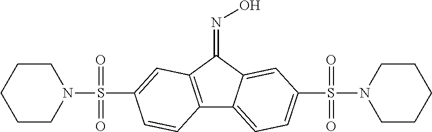 [SMILES: O=S(=O)(c1ccc2c(c1)C(=NO)c1cc(S(=O)(=O)N3CCCCC3)ccc1-2)N1CCCCC1]

### 2,7-bis(piperidin-1-yl-sulfonyl)-9H-fluoren-9-one oxime

b. Inhibitory Nucleic Acids that Hybridize to TAZ or YAP

Other means of inhibiting TAZ/YAP activity or gene expression can also be used in the methods of the invention. For example, a nucleic acid molecule complementary to at least a portion of a human TAZ/YAP encoding nucleic acid can be used to inhibit TAZ/YAP gene expression. Means for inhibiting gene expression using short RNA molecules, for example, are known. Among these are short interfering RNA (siRNA), small temporal RNAs (stRNAs), and micro-RNAs (miRNAs). Short interfering RNAs silence genes through a mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8(6):842-50 (2002); Morris et al., Science, 305(5688):1289-92 (2004); He and Hannon, Nat Rev Genet. 5(7):522-31 (2004).

“RNA interference,” a form of post-transcriptional gene silencing (“PTGS”), describes effects that result from the introduction of double-stranded RNA into cells (reviewed in Fire, A. Trends Genet 15:358-363 (1999); Sharp, P. Genes Dev 13:139-141 (1999); Hunter, C. Curr Biol 9:R440-R442 (1999); Baulcombe. D. Curr Biol 9:R599-R601 (1999); Vaucheret et al. Plant J 16: 651-659 (1998)). RNA interference, commonly referred to as RNAi, offers a way of specifically inactivating a cloned gene, and is a powerful tool for investigating gene function.

The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo.

In mammalian cells other than these, however, longer RNA duplexes provoked a response known as “sequence non-specific RNA interference,” characterized by the non-specific inhibition of protein synthesis.

Further studies showed this effect to be induced by dsRNA of greater than about 30 base pairs, apparently due to an interferon response. It is thought that dsRNA of greater than about 30 base pairs binds and activates the protein PKR and 2′,5′-oligonucleotide synthetase (2′,5′-AS). Activated PKR stalls translation by phosphorylation of the translation initiation factors eIF2α, and activated 2′,5′-AS causes mRNA degradation by 2′,5′-oligonucleotide-activated ribonuclease L. These responses are intrinsically sequence-nonspecific to the inducing dsRNA; they also frequently result in apoptosis, or cell death. Thus, most somatic mammalian cells undergo apoptosis when exposed to the concentrations of dsRNA that induce RNAi in lower eukaryotic cells.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes were too short to elicit sequence-nonspecific responses like apoptosis, yet they efficiently initiated RNAi. Many laboratories then tested the use of siRNA to knock out target genes in mammalian cells. The results demonstrated that siRNA works quite well in most instances.

For purposes of reducing the activity of TAZ/YAP, siRNAs to the gene encoding the TAZ/YAP can be specifically designed using computer programs. Illustrative nucleotide sequences encoding the amino acid sequences of the various YAP isoforms are known and published, e.g., in GenBank Accession Nos. NM_{—}001130145.2→NP_{—}001123617.1 yorkie homolog isoform 1; NM_{—}006106.4→NP_{—}006097.2 yorkie homolog isoform 2; NM_{—}001195044.1→NP_{—}001181973.1 yorkie homolog isoform 3; 3.NM_{—}001195045.1→NP_{—}001181974.1 yorkie homolog isoform 4. Furthermore, exemplary nucleotide sequences encoding the amino acid sequences of the various TAZ isoforms are known and published, e.g., in GenBank Accession Nos. NM_{—}001168278.1→NP_{—}001161750.1; 2.NM_{—}001168280.1→NP_{—}001161752.1; NM_{—}015472.4→NP_{—}056287.1; see also, Kanai, et al., The EMBO Journal (2000) 19(24):6778-6791.

Software programs for predicting siRNA sequences to inhibit the expression of a target protein are commercially available and find use. One program, siDESIGN from Dharmacon, Inc. (Lafayette, Colo.), permits predicting siRNAs for any nucleic acid sequence, and is available on the internet at dharmacon.com. Programs for designing siRNAs are also available from others, including Genscript (available on the internet at genscript.com/ssl-bin/app/rnai) and, to academic and non-profit researchers, from the Whitehead Institute for Biomedical Research found on the worldwide web at “jura.wi.mit.edu/pubint/http://iona.wi.mit.edu/siRNAext/.”

c. Inhibitors of Phosphodiesterase (PDE)

In various embodiments, the inhibitor of the Hippo-YAP signaling pathway is an inhibitor of a phosphodiesterase (PDE). The PDE inhibitor may or may not be selective, specific or preferential for cAMP. Illustrative PDEs that degrade cAMP include without limitation PDE3, PDE4, PDE7, PDE8 and PDE10. Illustrative cAMP selective hydrolases include PDE4, 7 and 8. Illustrative PDEs that hydrolyse both cAMP and cGMP include PDE1, 2, 3, 10 and 11. Isoenzymes and isoforms of PDEs are well known in the art. See, e.g., Boswell-Smith et al., Brit. J. Pharmacol. 147:S252-257 (2006), and Reneerkens, et al., Psychopharmacology (2009) 202:419-443, the contents of which are incorporated herein by reference.

In some embodiments, the PDE inhibitor is a non-selective inhibitor of PDE. Illustrative non-selective PDE inhibitors that find use include without limitation caffeine, theophylline, isobutylmethylxanthine, aminophylline, pentoxifylline, vasoactive intestinal peptide (VIP), secretin, adrenocorticotropic hormone, pilocarpine, alpha-melanocyte stimulating hormone (MSH), beta-MSH, gamma-MSH, the ionophore A23187, prostaglandin E1.

In some embodiments, the PDE inhibitor used specifically or preferentially inhibits PDE4. Illustrative inhibitors that selectively inhibit PDE4 include without limitation rolipram, roflumilast, cilomilast, ariflo, HT0712, ibudilast and mesembrine.

In some embodiments, the PDE inhibitor used specifically or preferentially inhibits a cAMP PDE, e.g., PDE4, PDE7 or PDE8. In some embodiments, the PDE inhibitor used inhibits a cAMP PDE, e.g., PDE1, PDE2, PDE3, PDE4, PDE7, PDE8, PDE10 or PDE11. Illustrative agents that inhibit a cAMP phosphodiesterase include without limitation rolipram, roflumilast, cilomilast, ariflo, HT0712, ibudilast, mesembrine, cilostamide, enoxamone, milrinone, siguazodan and BRL-50481.

In some embodiments, the PDE inhibitor used specifically inhibits PDE5. Illustrative inhibitors that selectively inhibit PDE5 include without limitation sildenafil, zaprinast, tadalafil, udenafil, avanafil and vardenafil.

Other means of inhibiting phosphodiesterase activity or gene expression can also be used in the methods of the invention. For example, a nucleic acid molecule complementary to at least a portion of a human phosphodiesterase gene (e.g., PDE3, PDE4, PDE7, PDE8 and PDE10) can be used to inhibit phosphodiesterase gene expression.

For purposes of reducing the activity of a phosphodiesterase enzyme, siRNAs to the gene encoding the phosphodiesterase can be specifically designed using computer programs. Illustrative nucleotide sequences encoding the amino acid sequences of the various phosphodiesterase isoforms are known and published, e.g., in GenBank Accession Nos., e.g., PDE1A (NM_{—}001003683.1→NP_{—}001003683.1 (isoform 2) and NM_{—}005019.3→NP_{—}005010.2 (isoform 1)); PDE1B (NM_{—}000924.3→NP_{—}000915.1 (isoform 1) and NM_{—}001165975.1→NP_{—}001159447.1 (isoform 2)); PDE2A (NM_{—}002599.3→NP_{—}002590.1 (isoform 1); NM_{—}001143839.2→NP_{—}001137311.1 (isoform 2) and NM_{—}001146209.1→NP_{—}001139681.1 (isoform 3)); PDE3A (NM_{—}000921.3→NP_{—}000912.3); PDE3B (NM_{—}000922.3→NP_{—}000913.2); PDE4A (NM_{—}001111307.1→NP_{—}001104777.1 (isoform 1); NM_{—}001111308.1→NP_{—}001104778.1 (isoform 2); NM_{—}001111309.1→NP_{—}001104779.1 (isoform 3); NM_{—}006202.2→NP_{—}006193.1 (isoform 4)); PDE4B (NM_{—}002600.3→NP_{—}002591.2 (isoform 1); NM_{—}001037341.1→NP_{—}001032418.1 (isoform 1); NM_{—}001037339.1→NP_{—}001032416.1 (isoform 2); NM_{—}001037340.1→NP_{—}001032417.1 (isoform 3)); PDE4C-1 (NM_{—}000923.3→NP_{—}000914.2); PDE4C-2 (NM_{—}001098819.1→NP_{—}001092289.1); PDE4C-3 (NM_{—}001098818.1→NP_{—}001092288.1); PDE4D1 (NM_{—}001197222.1→NP_{—}001184151.1); PDE4D2 (NM_{—}001197221.1→NP_{—}001184150.1); PDE4D3 (NM_{—}006203.4→NP_{—}006194.2); PDE4D4 (NM_{—}001104631.1→NP_{—}001098101.1); PDE4D5 (NM_{—}001197218.1→NP_{—}001184147.1); PDE4D6 (NM_{—}001197223.1→NP_{—}001184152.1); PDE4D7 (NM_{—}001165899.1→NP_{—}001159371.1); PDE4D8 (NM_{—}001197219.1→NP_{—}001184148.1); PDE5A (NM_{—}001083.3→NP_{—}001074.2 (isoform 1); NM_{—}033430.2→NP_{—}236914.2 (isoform 2); NM_{—}033437.3→NP_{—}246273.2 (isoform 3)); PDE7A (NM_{—}002603.2→NP_{—}002594.1 (isoform a); NM_{—}002604.2→NP_{—}002595.1 (isoform b)); PDE7B (NM_{—}018945.3→NP_{—}061818.1); PDE8A (NM_{—}002605.2→NP_{—}002596.1 (isoform 1); NM_{—}173454.1→NP_{—}775656.1 (isoform 2)); PDE8B (NM_{—}003719.3→NP_{—}003710.1 (isoform 1); NM_{—}001029854.2→NP_{—}001025025.1 (isoform 2); NM_{—}001029851.2→NP_{—}001025022.1 (isoform 3); NM_{—}001029853.2→NP_{—}001025024.1 (isoform 4); NM_{—}001029852.2→NP_{—}001025023.1 (isoform 5)).

d. Activators of Adenylyl Cyclase

In various embodiments, the inhibitor of the Hippo-YAP signaling pathway is an activator of adenylyl cyclase (AC). Activators of AC are known in the art and readily commercially available, e.g., from Sigma Aldrich (sigmaaldrich.com), EMD Millipore (emdmillipore.com), Merck Millipore (merckmillipore.com), and Tocris (tocris.com). Illustrative AC activators that can find use include without limitation forskolin and analogs thereof (e.g., forskolin, 6-Acetyl-7-deacetyl-forskolin, 7-Deacetyl-forskolin, 7-Deacetyl-6-(N-acetylglycyl)-forskolin, 7-Deacetyl-7-O-hemisuccinyl-forskolin, 7-Deacetyl-7-(O—N-methylpiperazino)-γ-butryl-Dihydrochloride forskolin); toxins that activate adenylate cyclase activity via ADP-ribosylation of G-proteins (e.g., pertussis toxin, cholera toxin, Pertussis Toxin A Protomer, Cholera Toxin A Subunit); NB001, NKH 477, pituitary adenylate cyclase activating polypeptide-38 (PACAP-38), pituitary adenylate cyclase activating polypeptide-27 (PACAP-27), ligands which activate adenylate cyclase activity via G-protein coupled receptors (e.g., Adenosine via A₂ receptors; Carbacyclin; Dopamine via D₁ receptors; Endothelin 1 via ET_{A} receptors; L-Epinephrine via β₁ and β₂ receptors; L-(−)-Epinephrine-(+)-bitartrate via β₁ and β₂ receptors; Glucagon; Isoproterenol; (±)-Octopamine; Parathyroid Hormone 1-34; Prostaglandin D₂; Prostaglandin E₁; Prostaglandin E₂ via EP₂ receptors; Prostaglandin I₂; and Vasopressin).

e. Modulators of G-Protein Coupled Receptors (GPCR)

In various embodiments, the inhibitor of the Hippo-YAP signaling pathway is an inhibitor of a Gα-protein selected from the group consisting of G12, G13, Gq, G11, Gi and Go or an antagonist of a G-protein-coupled receptor (GPCR) coupled to a Gα-protein selected from the group consisting of G12, G13, Gq, G11, Gi and Go. In some embodiments, the inhibitor of the Hippo-YAP signaling pathway is an activator of a Gs G-protein or an agonist of a G-protein-coupled receptor (GPCR) coupled to a Gs G protein. Inhibitors of G-proteins, including Gα-proteins, are known in the art and commercially available, e.g., from Tocris Bioscience (on the internet at tocris.com) and Novus Biological (on the internet at novusbio.com). G-protein inhibitors are also described, e.g., in Prevost, et al., Cancer Res (2006) 66:9227-9234 and Heximer, et al., Proc. Natl. Acad. Sci. USA (1997) 94:14389-14393. In various embodiments, the agent is an inhibitory nucleic acid (e.g., a small inhibitory RNA, a micro RNA, an antisense nucleic acid, a ribozyme) that inhibits the expression of Gα-protein selected from the group consisting of G12, G13, Gq, G11, Gi and Go.

G-protein-coupled receptors and the Gα-proteins to which they are coupled, are listed in Table 2. Agonists and antagonists of the listed G-protein-coupled receptors listed in Table 2 are well known. In various embodiments, the agonist or antagonist of the target G-protein-coupled receptor is an antibody or fragment thereof.

i. Antagonists of Lysophosphatidic Acid Receptor 1-5 (LPAR1-5)

In various embodiments, the inhibitor of the Hippo-YAP signaling pathway is an antagonist of lysophosphatidic acid receptor 1-5 (LPAR1-5). Antagonists of lysophosphatidic acid receptor 1-5 (LPAR1-5) are known in the art and find use. Illustrative antagonists of lysophosphatidic acid receptor 1-5 (LPAR1-5) include without limitation Ki16425, Ki16198, VPC 32183, N-P Serine PA, Anti-LPA Antibodies (e.g., Lpathomab), alpha-bromophosphonates (BrP-LPA) (Zhang, et al., Cancer Res. (2009) 69(13):5441-9); AM095 (sodium, {4′-[3-methyl-4-((R)-1-phenyl-ethoxycarbonylamino)-isoxazol-5-yl]-biphenyl-4-yl}-acetate) (Swaney, et al., J Pharmacol Exp Ther. (2011) 336(3):693-700); AM966 ((4′-{4-[(R)-1-(2-chloro-phenyl)-ethoxycarbonylamino]-3-methyl-isoxazol-5-yl}-biphenyl-4-yl)-acetic acid) (Swaney, et al., Br J Pharmacol. (2010) 160(7):1699-713); dual LPAR1/3 antagonist, VPC12249 (Gan, et al., Biochem Biophys Res Commun. (2011) 409(1):7-13); and those described in U.S. Patent Publication Nos. 20130072490, 20130072449, 20120289522, 20120258987, 20120196839, 20120015991, 20110301211, 20110301142, 20110301134, 20110196005, 20110082164; 20100311799, 20100152257 and 20090197835.

ii. Antagonists of Sphingosine 1-Phosphate (S1P) Receptors

In various embodiments, the inhibitor of the Hippo-YAP signaling pathway is an antagonist of sphingosine 1-phosphate (SIP) receptors. Antagonists of sphingosine 1-phosphate (SIP) receptors are known in the art and find use. Illustrative antagonists of sphingosine 1-phosphate (SIP) receptors include without limitation VPC 23019, Anti-SIP Antibodies (e.g., Sphingomab), TY-52156 (1-(4-chlorophenylhydrazono)-1-(4-chlorophenylamino)-3,3-dimethyl-2-butanone) (Murakami, et al., Mol Pharmacol. (2010) 77(4):704-13); W146 (Tarrasón, et al., Int Immunopharmacol. (2011) 11(11):1773-9); JTE-013 (Li, et al., J Neurophysiol. (2012) 108(5):1473-83); and those described in U.S. Patent Publication Nos. 20130109669, 20130079290, 20130065954, 20120190694, 20120220552, 20120142745, 20120142740, 20120142739, 20120142736, 20120142664, 20120142663, 20120142662, 20120142661, 20120142642, 20120142640, 20120142639, 20120129906, 20120129829 and 20120129814.

iii. Antagonists of Coagulation Factor II (Thrombin) Receptors

In various embodiments, the inhibitor of the Hippo-YAP signaling pathway is an antagonist of coagulation factor II (thrombin) receptors. Antagonists of coagulation factor II (thrombin) receptors are known in the art and find use. Illustrative antagonists of coagulation factor II (thrombin) receptors include without limitation Vorapaxar (Tricoci, et al., N Engl J Med. (2012) 366(1):20-33); FR 171113, FSLLRY-NH2, RWJ 56110, SCH-530348 (Oestreich, Curr Opin Investig Drugs. (2009) 10(9):988-96); and those described in U.S. Patent Publication Nos. 20120214845, 20120157403, 20120028976, 20110301112, 20110105490, 20090076088, 20090069383, 20080090830, 20080085923, 20080004318, 20070232635, 20070149518, 20060223808, 20060166897, 20060009396 and 20050267155.

iv. Antagonists of Estrogen Receptor 1 (GPR30)

In various embodiments, the inhibitor of the Hippo-YAP signaling pathway is an antagonist of estrogen receptor 1 (GPR30). Antagonists of estrogen receptor 1 (GPR30) are known in the art and find use. Illustrative antagonists of estrogen receptor 1 (GPR30) include without limitation G-15, G-36 (Tocris), Enclomiphene (Androxal) (Hill, et al., IDrugs. (2009) 12(2):109-19); Estriol (E3) (Lappano, et al., Mol Cell Endocrinol. (2010) 320(1-2):162-70); and those described in U.S. Patent Publication Nos. 20130137746, 20120015919, 20120108571, 20110237625, 20110201555, 20100168188, 20090326018, 20090111855, 20080286265, 20080221179, 20070142379, and 20060040917.

v. Antagonists of Frizzled Homolog D4

In various embodiments, the inhibitor of the Hippo-YAP signaling pathway is an antagonist of frizzled homolog D4. Antagonists of frizzled homolog D4 are known in the art and find use. Illustrative antagonists of frizzled homolog D4 include without limitation secreted Frizzled-related protein (von Marschall, et al., Biochem Biophys Res Commun. (2010) 400(3):299-304), Dickkopf proteins and those described in U.S. Patent Publication Nos. 20120202749, 20120027778, 20120014876, 20080086002 and 20080038272.

vi. Antagonists of Endothelin Receptors

In various embodiments, the promoter of the Hippo-YAP signaling pathway is an antagonist of endothelin receptors. Antagonists of endothelin receptors are known in the art and find use. Illustrative antagonists of endothelin receptors include without limitation sitaxentan, ambrisentan, atrasentan, BQ-123, zibotentan, bosentan, macitentan, tezosentan, clazosentan (Macdonald, et al., Lancet Neurol. (2011) 10(7):618-25); and those described in U.S. Patent Publication Nos. 20110263854, 20110082151, 20100093758, 20100063076, 20090263472, 20080004298, 20070173520, 20040077670, 20040063731 and 20040034076.

vii. Antagonists of CXCR2

In various embodiments, the promoter of the Hippo-YAP signaling pathway is an antagonist of CXCR2. Antagonists of CXCR2 are known in the art and find use. Illustrative antagonists of CXCR2 include without limitation BMS CCR2 22, INCB 3284 dimesylate, SB 265610, and those described in U.S. Patent Publication Nos. 20120046243, 20110184177, 20110009429, 20100210593, 20100152205, 20090258906, 20090215827 and 20070248594.

viii. Antagonists of CXCR4

In various embodiments, the promoter of the Hippo-YAP signaling pathway is an antagonist of CXCR4. Antagonists of CXCR4 are known in the art and find use. Illustrative antagonists of CXCR4 include without limitation AMD 3100 octahydrochloride, AMD 3465 hexahydrobromide, IT1t dihydrochloride, and those described in U.S. Patent Publication Nos. 20130035347, 20130029902, 20120294803, 20110294156, 20110269686, 20110250165, 20110086027, 20100130409, 20090099194 and 20080227799.

f. Actin Disrupting Agents

In various embodiments, the inhibitor of the Hippo-YAP signaling pathway is an actin disrupting agent. Actin disrupting agents are known in the art. Illustrative actin disrupting agents include without limitation Cytochalasin A, Cytochalasin B, Cytochalasin C, Cytochalasin D, Cytochalasin E, Cytochalasin F, Cytochalasin G, Cytochalasin H, Cytochalasin I, Cytochalasin J, Latrunculin A, Latrunculin B, Swinholide A, Misakinolide A, Bistheonelide A, Scytophycin A, Scytophycin B, Scytophycin D, Scytophycin E, 19-0-Demethylscytophycin C, 6 Hydroxyscytophycin B, 6-Hydroxy-7-o-methylscytophycin E and tolytoxin, Mycalolide A, Mycalolide B, Mycalolide C, secomycalolide A and 30-hydroxymycalolide A, Halichondramide, (19Z)-halichondramide, kabiramides B, kabiramides C, kabiramides D, kabiramides G, kabiramides J, kabiramides K, ulapualide A, jaspamide, Dihydrohalichondramide, Aplyronine A, Aplyronine B, Aplyronine C, Pectenotoxin 2, Pectenotoxin 6, and Migrastatin.

### 4. Formulation and Administration

In various embodiments, the compositions of the invention comprise an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof). The an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) can be formulated together (e.g., as a mixture) or separately, e.g., with the anti-inflammatory agent and/or the chemotherapeutic agent. The an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) can be prepared and administered in a wide variety of oral, parenteral and topical dosage forms. In preferred forms, compositions for use in the methods of the present invention can be administered orally, by injection, that is, intravenously, intramuscularly, intracutaneously, subcutaneously, intraduodenally, or intraperitoneally. The compositions can also be administered by inhalation, for example, intranasally. Additionally, the compositions can be administered transdermally. Accordingly, in some embodiments, the methods of the invention permit administration of compositions comprising a pharmaceutically acceptable carrier or excipient, an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof), or a pharmaceutically acceptable salt of the inhibitor.

Compositions are provided that contain therapeutically effective amounts of the compound. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

The agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) can be administered in the “native” form or, if desired, in the form of salts, esters, amides, prodrugs, derivatives, and the like, provided the salt, ester, amide, prodrug or derivative is suitable pharmacologically effective, e.g., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of formulating such derivatives are known to those of skill in the art. For example, the disulfide salts of a number of delivery agents are described in PCT Publication WO 2000/059863 which is incorporated herein by reference. Similarly, acid salts of therapeutic peptides, peptoids, or other mimetics, and can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, orotic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the active agents herein include halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. In certain embodiments basic salts include alkali metal salts, e.g., the sodium salt, and copper salts.

For the preparation of salt forms of basic drugs, the pKa of the counterion is preferably at least about 2 pH lower than the pKa of the drug. Similarly, for the preparation of salt forms of acidic drugs, the pKa of the counterion is preferably at least about 2 pH higher than the pKa of the drug. This permits the counterion to bring the solution's pH to a level lower than the pHmax to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (e.g., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

In various embodiments preparation of esters typically involves functionalization of hydroxyl and/or carboxyl groups that are present within the molecular structure of the active agent. In certain embodiments, the esters are typically acyl-substituted derivatives of free alcohol groups, e.g., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

About 1 to 1000 mg of an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) or a physiologically acceptable salt or ester is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 1-1000 mg, 2-800 mg, 5-500 mg, 10-400 mg, 50-200 mg, e.g., about 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 200 mg, 300 mg, 400 mg, 500 mg, 600 mg, 700 mg, 800 mg, 900 mg or 1000 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, the compound is mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween™, and dissolution in aqueous sodium bicarbonate. Derivatives of the compounds, such as salts or prodrugs may also be used in formulating effective pharmaceutical compositions.

The concentration of the compound is effective for delivery of an amount upon administration that lessens or ameliorates at least one symptom of the disorder for which the compound is administered and/or that is effective in a prophylactic context. Typically, the compositions are formulated for single dosage (e.g., daily) administration.

The compounds may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder. A therapeutically or prophylactically effective dose can be determined by first administering a low dose, and then incrementally increasing until a dose is reached that achieves the desired effect with minimal or no undesired side effects.

In various embodiments, the compounds and/or analogs thereof can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compounds. The containers are preferably adapted for the desired mode of administration, including, but not limited to tablets, gel capsules, sustained-release capsules, and the like for oral administration; depot products, pre-filled syringes, ampules, vials, and the like for parenteral administration; and patches, medipads, creams, and the like for topical administration.

The concentration and/or amount of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound can be provided in a formulation that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

In various embodiments, the tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

An agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) can be introduced into the bowel by use of a suppository. As is known in the art, suppositories are solid compositions of various sizes and shapes intended for introduction into body cavities. Typically, the suppository comprises a medication, which is released into the immediate area from the suppository. Typically, suppositories are made using a fatty base, such as cocoa butter, that melts at body temperature, or a water-soluble or miscible base, such as glycerinated gelatin or polyethylene glycol.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The dosage of the specific compounds depends on many factors that are well known to those skilled in the art. They include for example, the route of administration and the potency of the particular compound. An exemplary dose is from about 0.001 μg/kg to about 100 mg/kg body weight of the mammal. Doses of chemotherapeutic agents are known in the art, and can be found, e.g., in the published literature and in reference texts, e.g., the Physicians' Desk Reference, 65th Ed., 2011, Thomson Healthcare or Brunton, et al., Goodman & Gilman's The Pharmacological Basis of Therapeutics, 12th edition, 2010, McGraw-Hill Professional). Because of the cooperative action between An agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) and/or the chemotherapeutic agent, one or both of the co-administered agents can be administered at a sub-therapeutic dose.

Determination of an effective amount is well within the capability of those skilled in the art, especially in light of the detailed disclosure provided herein. Generally, an efficacious or effective amount of a combination of one or more polypeptides of the present invention is determined by first administering a low dose or small amount of a polypeptide or composition and then incrementally increasing the administered dose or dosages, adding a second or third medication as needed, until a desired effect of is observed in the treated subject with minimal or no toxic side effects. Applicable methods for determining an appropriate dose and dosing schedule for administration of a combination of the present invention are described, for example, in Goodman and Gilman's The Pharmacological Basis of Therapeutics, 12th Edition, 2010, supra; in a Physicians' Desk Reference (PDR), 65^{th} Edition, 2011; in Remington: The Science and Practice of Pharmacy, 21^{st} Ed., 2005, supra; and in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, each of which are hereby incorporated herein by reference.

### 5. Combination Therapies

a. Immunotherapy

An agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) and/or a chemotherapeutic agent can be further co-administered with one or more therapeutic antibodies as combination therapies. In various embodiments, an antibody or antibody fragment that binds to a surface tumor-associated antigen of a cancer cell can be used to target delivery of an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) to the cancer cell or the tumor. An agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) can be conjugated to the antibody using methods well known in the art and delivered to the cancer cell or tumor as “cargo.”

Examples of therapeutic antibodies that can be co-administered with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) include but are not limited to HERCEPTIN™ (Trastuzumab) (Genentech, CA) which is a humanized anti-HER2 monoclonal antibody for the treatment of patients with metastatic breast cancer; REOPRO™ (abciximab) (Centocor) which is an anti-glycoprotein IIb/IIIa receptor on the platelets for the prevention of clot formation; ZENAPAX™ (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope); IgG antibody (ImClone System); IMC-C225 which is a chimeric anti-EGFR IgG antibody; VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); ICM3 which is a humanized anti-ICAM3 antibody (ICOS Pharm); IDEC-114 which is a primate anti-CD80 antibody (IDEC Pharm/Mitsubishi); ZEVALIN™ which is a radiolabelled murine anti-CD20 antibody (IDEC/Schering AG); IDEC-131 which is a humanized anti-CD40L antibody (IDEC/Eisai); IDEC-151 which is a primatized anti-CD4 antibody (IDEC); IDEC-152 which is a primatized anti-CD23 antibody (IDEC/Seikagaku); SMART anti-CD3 which is a humanized anti-CD3 IgG (Protein Design Lab); 5G1.1 which is a humanized anti-complement factor 5 (CS) antibody (Alexion Pharm); D2E7 which is a humanized anti-TNF-α antibody (CATIBASF); CDP870 which is a humanized anti-TNF-α Fab fragment (Celltech); IDEC-151 which is a primatized anti-CD4 IgG1 antibody (IDEC Pharm/SmithKline Beecham); MDX-CD4 which is a human anti-CD4 IgG antibody (Medarex/Eisai/Genmab); CDP571 which is a humanized anti-TNF-α IgG4 antibody (Celltech); LDP-02 which is a humanized anti-α4,7 antibody (LeukoSite/Genentech); OrthoClone OKT4A which is a humanized anti-CD4 IgG antibody (Ortho Biotech); ANTOVA™ which is a humanized anti-CD40L IgG antibody (Biogen); ANTEGREN™ which is a humanized anti-VLA-4 IgG antibody (Elan); and CAT-152 which is a human anti-TGF-β₂ antibody (Cambridge Ab Tech).

Therapeutic antibodies that specifically bind to a tumor-associated antigen (“TAA”) find use. Examples of known TAAs include without limitation, melanoma associated antigens (MAGE-1, MAGE-3, TRP-2, melanosomal membrane glycoprotein gp100, gp75 and MUC-1 (mucin-1) associated with melanoma); CEA (carcinoembryonic antigen) which can be associated, e.g., with ovarian, melanoma or colon cancers; folate receptor alpha expressed by ovarian carcinoma; free human chorionic gonadotropin beta (hCGβ) subunit expressed by many different tumors, including but not limited to myeloma; HER-2/neu associated with breast cancer; encephalomyelitis antigen HuD associated with small-cell lung cancer; tyrosine hydroxylase associated with neuroblastoma; prostate-specific antigen (PSA) associated with prostate cancer; CA125 associated with ovarian cancer; and the idiotypic determinants of a B cell lymphoma can generate tumor-specific immunity (attributed to idiotype-specific humoral immune response). Moreover, antigens of human T cell leukemia virus type 1 have been shown to induce specific CTL responses and antitumor immunity against the virus-induced human adult T cell leukemia (ATL). See, e.g., Haupt, et al., Experimental Biology and Medicine (2002) 227:227-237; Ohashi, et al., Journal of Virology (2000) 74(20):9610-9616. Other TAAs are known and find use for co-administration with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof).

b. Radiation

An agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) can be administered in conjunction with radiological procedures. A variety of radiological procedures are available for disease treatments. Any of the procedures know by one of skill can be combined with the polypeptides of the present invention for treatment of a patient. Radiological procedures comprise treatment using radiation therapy to damage cellular DNA. The damage to the cellular DNA can be caused by a photon, electron, proton, neutron, or ion beam directly or indirectly ionizing the atoms which make up the DNA chain. Indirect ionization occurs due to the ionization of water, forming free radicals, notably hydroxyl radicals, which then subsequently damage the DNA. In the most common forms of radiation therapy, the majority of the radiation effect is through free radicals. Due to cellular DNA repair mechanisms, using agents that induce double-strand DNA breaks, such as radiation therapies, has proven to be a very effective technique for cancer therapy. Cancer cells are often undifferentiated and stem cell-like, such cells reproduce more rapidly and have a diminished ability to repair sub-lethal damage compared healthy and more differentiated cells. Further, DNA damage is inherited through cell division, leading to an accumulation of damage to the cancer cells, inducing slower reproduction and often death.

The amount of radiation used in radiation therapy procedure is measured in gray (Gy), and varies depending on the type and stage of cancer being treated and the general state of the patient's health. The dosage range can also be affected by cancer type, for example, the typical curative dosage for a solid epithelial tumor ranges from 60 to 80 Gy, while the dosage for lymphoma ranges from 20 to 40 Gy.

Preventative (adjuvant) doses can also be employed and typically range from 45 to 60 Gy administered in 1.8 to 2 Gy fractions (e.g., for breast, head and neck cancers). Many other factors are well-known and would be considered by those of skill when selecting a dose, including whether the patient is receiving other therapies (such as for example, but not limited to administration of an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof), administration of chemotherapies and the like), patient co-morbidities, timing of radiation therapy (for example, whether radiation therapy is being administered before or after surgery), and the degree of success of any surgical procedures.

Delivery parameters of a prescribed radiation dose can be determined during treatment planning by one of skill. Treatment planning can be performed on dedicated computers using specialized treatment planning software. Depending on the radiation delivery method, several angles or sources may be used to sum to the total necessary dose. Generally, a plan is devised that delivers a uniform prescription dose to the tumor and minimizes the dosage to surrounding healthy tissues.

c. Surgery

An agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) can be administered in conjunction with surgical removal or debulking of tumors, e.g., bone marrow transplantation. Any of the procedures known by one of skill can be combined with the administration of an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) for treatment and/or prevention of cancer in a patient.

### 6. Methods of Monitoring

A variety of methods can be employed in determining efficacy of therapeutic and prophylactic treatment with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof). Generally, efficacy is the capacity to produce an effect without significant toxicity. Efficacy indicates that the therapy provides therapeutic or prophylactic effects for a given intervention (examples of interventions can include by are not limited to administration of a pharmaceutical formulation, employment of a medical device, or employment of a surgical procedure). Efficacy can be measured by comparing treated to untreated individuals or by comparing the same individual before and after treatment. Efficacy of a treatment can be determined using a variety of methods, including pharmacological studies, diagnostic studies, predictive studies and prognostic studies. Examples of indicators of efficacy include but are not limited to inhibition of tumor cell growth and promotion of tumor cell death.

The efficacy of an anti-cancer treatment can be assessed by a variety of methods known in the art. An agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) can be screened for prophylactic or therapeutic efficacy in animal models in comparison with untreated or placebo controls. An agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) identified by such screens can be then analyzed for the capacity to induce tumor cell death or enhanced immune system activation. For example, multiple dilutions of sera can be tested on tumor cell lines in culture and standard methods for examining cell death or inhibition of cellular growth can be employed. (See, e.g., Maniatis, et al., Molecular Cloning: A Laboratory Manual, Cold Spring Harbor Lab., New York, 1982; Ausubel, et al. Editor, Current Protocols in Molecular Biology, USA, 1984-2008; and Ausubel, et al. Editor, Current Protocols in Molecular Biology, USA, 1984-2008; Bonifacino, et al., Editor, Current Protocols in Cell Biology, USA, 2010; all of which are incorporated herein by reference in their entirety.)

The methods of the present invention provide for detecting inhibition disease in patient suffering from or susceptible to various cancers. A variety of methods can be used to monitor both therapeutic treatment for symptomatic patients and prophylactic treatment for asymptomatic patients.

Monitoring methods entail determining a baseline value of a tumor burden in a patient before administering a dosage of an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof), and comparing this with a value for the tumor burden after treatment, respectively.

With respect to therapies using An agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof), a significant decrease (i.e., greater than the typical margin of experimental error in repeat measurements of the same sample, expressed as one standard deviation from the mean of such measurements) in value of the tumor burden signals a positive treatment outcome (i.e., that administration of an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) has blocked or inhibited, or reduced progression of tumor growth and/or metastasis).

In other methods, a control value of tumor burden (e.g., a mean and standard deviation) is determined from a control population of individuals who have undergone successful treatment with an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof). Measured values of tumor burden in a patient are compared with the control value. If the measured level in a patient is not significantly different (e.g., less than one standard deviation) from the control value, treatment can be discontinued. If the tumor burden level in a patient is significantly above the control value, continued administration of agent is warranted.

In other methods, a patient who is not presently receiving treatment but has undergone a previous course of treatment is monitored for tumor burden to determine whether a resumption of treatment is required. The measured value of tumor burden in the patient can be compared with a value of tumor burden previously achieved in the patient after a previous course of treatment. A significant increase in tumor burden relative to the previous measurement (i.e., greater than a typical margin of error in repeat measurements of the same sample) is an indication that treatment can be resumed. Alternatively, the value measured in a patient can be compared with a control value (mean plus standard deviation) determined in a population of patients after undergoing a course of treatment. Alternatively, the measured value in a patient can be compared with a control value in populations of prophylactically treated patients who remain free of symptoms of disease, or populations of therapeutically treated patients who show amelioration of disease characteristics. In all of these cases, a significant increase in tumor burden relative to the control level (i.e., more than a standard deviation) is an indicator that treatment should be resumed in a patient.

The tissue sample for analysis is typically blood, plasma, serum, mucous, tissue biopsy, tumor, ascites or cerebrospinal fluid from the patient. The sample can be analyzed for indication of neoplasia. Neoplasia or tumor burden can be detected using any method known in the art, e.g., visual observation of a biopsy by a qualified pathologist, or other visualization techniques, e.g., radiography, ultrasound, magnetic resonance imaging (MRI).

Further, the level of immune system activity in conjunction with tumor burden in a patient before administering a dosage of an agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) can be compared this with a value for the immune system activity in conjunction with tumor burden after treatment, again respectively.

With respect to therapies involving enhanced immune system activity, a significant increase (i.e., greater than the typical margin of experimental error in repeat measurements of the same sample, expressed as one standard deviation from the mean of such measurements) in value of immune response signals a positive treatment outcome (i.e., that administration of An agent that inhibits the Hippo-YAP signaling pathway (e.g., an inhibitor of TAZ/YAP, an activator of PKA (e.g., an adenylyl cyclase (AC) activator and/or a phosphodiesterase (PDE) inhibitor), an inhibitor of G12, G13, Gq, G11, Gi and Go, an activator of Gs, and mixtures thereof) has achieved or augmented an immune response). Immune response signals can include but are not limited to for example assessing the enhancement of the lymphoma-specific cytotoxic effect of human peripheral blood mononuclear cells (PBMCs). If the value for the immune response signal does not change significantly, or decreases, a negative treatment outcome is indicated. In general, patients undergoing an initial course of treatment with an immunogenic agent are expected to show an increase in immune response activity with successive dosages, which eventually reaches a plateau. Administration of an agent is often continued while the immune response is increasing. Once a plateau is obtained, that is an indicator if the treatment is solely for the immune the administration of the treatment can be discontinued or reduced in dosage or frequency.

### EXAMPLES

The following examples are offered to illustrate, but not to limit the claimed invention.

### Example 1

### Regulation of the Hippo-YAP Pathway by G-Protein Coupled Receptor Signaling

### Materials and Methods

Cell Culture.

All cell lines were maintained at 37° C. with 5% CO₂. HEK293A, HEK293T, HeLa, RC3, SK-Mel-28, SF268, MDA-MB-231, and U2OS cells were cultured in DMEM (Invitrogen) containing 10% FBS (Omega Scientific) and 50 μg/mL penicillin/streptomycin (P/S). Primary hepatocytes were isolated from 12 weeks old male mice using standard protocol and incubated in complete DMEM medium. MCF10A cells were cultured in DMEM/F12 (Invitrogen) supplemented with 5% horse serum (Invitrogen), 20 ng/mL EGF, 0.5 μg/mL hydrocortisone, 10 μg/mL insulin, 100 ng/mL cholera toxin, and 50 μg/mL P/S. For serum starvation, cells were incubated in DMEM or DMEM/F12 without other supplements.

Chemicals.

The following chemicals were used in this study: C3 (Cytoskeleton Inc.), Ki16425 (Cayman Chemical), Torin1 (from Dr. David Sabatini). Lipids were purchased from Avanti Polar Lipids and all other chemicals were purchased from Sigma Aldrich or Tocris.

Transfection.

Cells were transfected with plasmid DNA using PolyJet™ DNA In Vitro Tranfection Reagent (Signagen Laboratories) according to manufacturer's instruction. G protein, LPA or S1P receptor plasmids were provided by Dr. Rick Neubig or purchased from Missouri S&T cDNA Resource Center. siRNAs were delivered into cells using RNAiMAX (Invitrogen) according to manufacturer's instructions.

Lipid Extraction.

FBS (100 μl) was mixed with 600 μl of chloroform, methanol or a mixture of chloroform and methanol at 2:1 or 1:2 ratios. After setting at room temperature for 30 min, layers were separated by centrifugation at 13,000 rpm for 5 min. Aqueous layer were removed, and organic solvent was evaporated. Materials extracted were dissolved in 100 μl of fatty acid-free BSA (FAF, 2 mg/ml) and used to treat cells (100× diluted, equivalent to 1% FBS). In some extractions, 0.05 N HCl or NaOH (final concentration) was added from 1 N stock. In one experiment, 200 μl of H₂O was added into the organic phase (CM 2:1) to repeat the extraction procedure.

Immunoprecipitation.

Cells were lysed using mild lysis buffer (50 mM HEPES at pH 7.5, 150 mM NaCl, 1 mM EDTA, 1% NP-40, 10 mM pyrophosphate, 10 mM glycerophosphate, 50 mM NaF, 1.5 mM Na₃VO₄, protease inhibitor cocktail [Roche], 1 mM PMSF). Cell lysates were centrifuged for 10 min at 4° C., and supernatants were used for immunoprecipitation. YAP (Bethyl Laboratories) antibody was mixed with the supernatant for 1 h, and protein A-agarose beads were added in for 1 h. Immunoprecipitates were washed four times with lysis buffer, and proteins were eluted with SDS-PAGE sample buffer. For kinase assay, MST1, Lats1 or HA-Lats2 was immunoprecipitated similarly using MST1, Lats1 (Cell Signaling) or HA (Covance) antibodies.

Immunoblotting.

Immunoblotting was performed using standard protocol. Antibodies for pYAP (S127), pYAP(S381/384), pMST1/2, TAZ, pERK, ERK1/2, Lats1, MST1, pAKT (S473), pS6K, S6K were from Cell Signaling, antibodies for YAP (H-125), CTGF, Cyr61, Gq/11, G13 and 14-3-3θ were from Santa Cruz, TEAD1 antibody was from BD biosciences, pTAZ (S89) antibody was in-house raised, GAPDH antibody was a gift from Dr. Yan Luo. The phos-tag reagents were purchased from Wako Chemicals, and gels containing phos-tag were prepared according to manufacture's instructions. YAP proteins can be separated into multiple bands in the presence of phos-tag depending on differential phosphorylation levels, with phosphorylated proteins run migrate slower. YAP-5SA with all Lats kinase targeting serine residues mutated migrate at a similar speed as dephosphorylated YAP, suggesting that phosphorylation at Lats targeting sites is responsible for slower migrating bands on phos-tag-containing gels.

Immunofluorescence Staining.

HEK293A or MCF10A cells were seeded on coverslips. After treatment, cells were fixed with 4% paraformaldehyde-PBS for 15 min and permeabilized with 0.1% Triton X-100 in TBS. After blocking in 3% FBS in PBS for 30 min, cells were incubated with primary antibodies overnight at 4° C. After three washes with PBS, cells were incubated with Alexa Fluor 488- or 555-conjugated secondary antibodies (Invitrogen, 1:1000 dilution) for 2 h at room temperature. Slides were then washed three times and mounted. Immunofluorescence was detected using Olympus confocal microscopy. Primary antibodies used were: YAP (H-125) and YAP (63.7) from Santa Cruz, HA from Covance, β-catenin from BD biosciences. Phalloidin alexa 488 was used to stain actin filaments. For paraffin-embedded tissues from control or LPA receptor transgenic mammary glands, 5 μm sections were prepared and subjected for immunostaining Following deparaffinization and hydration, slides were heated in sub-boiling buffer (10 mM sodium citrate, pH 6.0) for 15 min for antigen retrieval. TAZ antibody (H-70) from Santa Cruz was used to as primary antibody.

Kinase Assay.

Following immunoprecipitation, protein beads were washed once with wash buffer (40 mM HEPES, 200 mM NaCl) and once with kinase assay buffer (30 mM HEPES, 50 mM potassium acetate, 5 mM MgCl₂). The immunoprecipitated Mst1 was subjected to a kinase assay in the presence of 500 μM cold ATP, 10 μCi [γ-³²P] ATP, and 1 μg of GST-Mob expressed and purified from Escherichia coli as substrate. The reaction mixtures were incubated for 30 min at 30° C., terminated with SDS sample buffer, and subjected to SDS-PAGE and autoradiography. Lats1 or HA-Lats2 kinase assays were performed similarly but using GST-YAP as substrates in the absence of [γ-³²P] ATP. The phosphorylation of GST-YAP at S127 was determined by immunoblotting using pYAP antibody.

RNA Extraction, Reverse Transcription and Real-Time PCR.

Following various treatments, cells were washed with cold phosphate-buffered saline and subjected to RNA extraction using an RNeasy Plus mini kit (Qiagen). RNA samples (1 μg) were reverse-transcribed to complementary DNA using iScript reverse transcriptase (Bio-Rad). Complementary DNA was then diluted and used for quantification (with β-actin gene as a control) by real-time PCR, which was performed using KAPA SYBR FAST qPCR master mix (Kapa Biosystems) and the 7300 real-time PCR system (Applied Biosystems). Primer pairs used in this study are:

LPAR1:
GTGTGGGCTGGAACTGTATCTG/TAGTCCTCTGGCGAACATAG
LPAR2:
GGCCAGTGCTACTACAACGAGACC/TGGAGGCGATGGCTGCTATGAC
LPAR3:
CCTGGTGGTTCTGCTCCTCGAC/GTGCCATACATGTCCTCGTCCTTG
LPAR4:
ATTGAAGTTGTTGGGTTTATCAT/GCACAAGGTGATTGGGTACAT
LPAR5:
CCTGGCGGCGGTGGTCTACTCGTC/
GACCGCCAGCGTGCTGTTGTAGGG
β-actin:
GCCGACAGGATGCAGAAGGAGATCA/
AAGCATTTGCGGTGGACGATGGA
CTGF:
CCAATGACAACGCCTCCTG/TGGTGCAGCCAGAAAGCTC
Cyr61:
AGCCTCGCATCCTATACAACC/TTCTTTCACAAGGCGGCACTC
ANKRD1:
CACTTCTAGCCCACCCTGTGA/CCACAGGTTCCGTAATGATTT
TAGLN:
CCCGAGAACCCACCCTCCA/AAAGCCATCAGGGTCCTCTGC
EDN1:
TGTGTCTACTTCTGCCACCT/CCCTGAGTTCTTTTCCTGCTT
PPP1ReB:
GGACACGTTCTCCTTCGAC/AGATTTTAACTCAGCCCGGAT
EGR3:
GCAGCGACCACCTCACCAC/CCGCCTTCTTCTCCTTTTGCT
EGR4:
CGACGAGCTCAATCGCCACCT/GCCGCACACGTCGCAAGCAA

RNA Interference.

Protein expression silencing was done by either lentiviral shRNA or siRNA. Mission shRNA (Sigma Aldrich) plasmids were used together with pMD2.G and psPAX2 to produce lentivirus in 293T cells. For some experiments, ON-TARGET plus SMARTpool siRNA for YAP, TAZ, Gq, G11, G12, G13 or non-targeting control (Dharmacon) were used to repress YAP or TAZ expression. The TRC numbers for shRNA plasmids were shown below:

YAP, TRCN0000300325

TAZ, TRCN0000370007

LPA1, TRCN0000011366

LPA3, TRCN0000011390

Cell Proliferation Assay.

HEK293A cells (expressing control shRNA or YAP/TAZ shRNA, 2×10⁵) in serum-free media were maintained in the presence or absence of 10 μM LPA for 1, 2 or 3 day. Cell numbers were determined daily using a cell counter (Bio-Rad). LPA was replenished every day.

Cell Migration Assay.

Cell migration assay was performed using BD Falcon™ Cell culture inserts for 24-well plates with 8.0 μm pores filter, and the filter was pre-coated with 20 μg/ml Fibronectin. MCF10A cells transfected with control siRNA or YAP/TAZ siRNA were serum-starved for 24 h and then seeded into the upper chamber of the insert (2×10⁵ cells/well) in serum-free media, and lower chamber was filled with media containing 20% mTeSR1 (STEMCELL Technologies) with or without 1 μM LPA. After 24 h, cells were fixed using 4% paraformaldehyde and stained using 0.05% crystal violet. Cells in upper chamber were carefully removed, and cells migrated through the filter were assessed by photography. For quantification, crystal violet was extracted and the absorbance at 560 nm was measured.

Epinephrine Treatment in Mice.

A protocol for epinephrine intraperitoneal (IP) injection in mice was approved by UCSD Institutional Animal Care and Use Committee. Fed male mice at 12 weeks of age were anesthetized. Control animals received an IP injection of propranolol (4 mg/100 g body weight) for 15 min. The test animals IP injected with epinephrine (75 μg/100 g body weight) for 15 min. Blood glucose levels were determined before and after the drug injection using an Accu-Check glucometer (Roche). The mice were sacrificed rapidly by cervical dislocation and the heart was harvested and immediately frozen in liquid nitrogen. Samples were stored at −80° C. until use. Frozen tissues were pulverized in liquid nitrogen. All of the subsequent steps were performed at 4° C. Powdered tissue samples were homogenized in 10 volumes (weight/volume) of buffer containing 50 mM Tris-HCl pH 7.6, 10 mM EDTA, 2 mM EGTA, 100 mM NaF, Protease inhibitor cocktail (1 mM Pefabloc, 1 mM benzamidine HCl, 1 μM leupeptin, and 1 μM E64), 1 mM PMSF, 0.2% Tritonx 100, 1 mM Na₃VO₄, 20 mM beta-glycerophosphate, 1 mM sodium pyrophosphate, and 50 nM calyculin A, using a tissue tearor. Homogenates were then centrifuged at 3,800 g for 10 min twice, and approximately 10 μg of protein, determined by Bradford, of the resultant supernatants were used for Western blot analysis.

### Results

### Serum Induces Dephosphorylation and Nuclear Localization of YAP

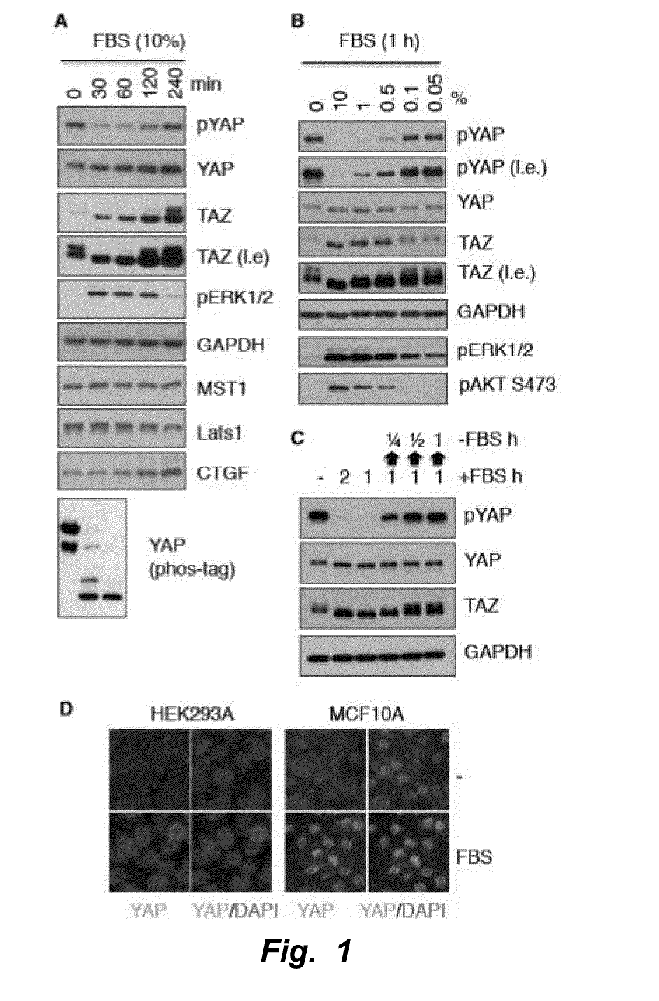

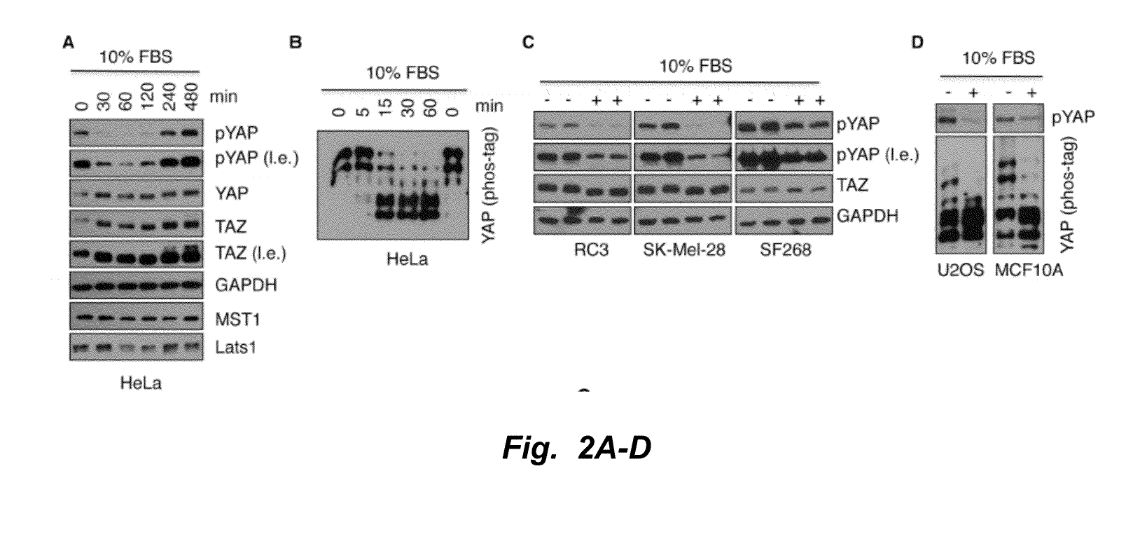

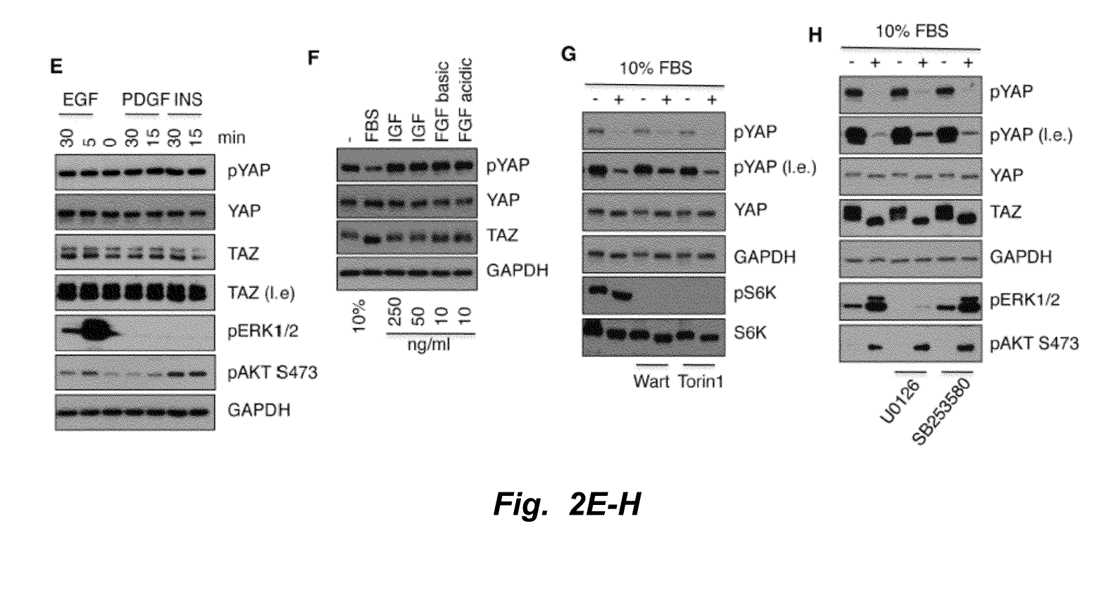

Phosphorylation of YAP S127 by Lats1/2 results in YAP cytoplasmic localization and therefore YAP inactivation (Dong et al., 2007; Hao et al., 2008; Zhao et al., 2007). In search of signals that might regulate YAP phosphorylation, we found that in HEK293A cells, YAP was highly phosphorylated under serum starvation conditions and addition of serum resulted in a rapid decrease in YAP phosphorylation as determined by a phospho-YAP antibody (pS127) and differential migration on phos-tag containing gels (FIG. 1A). This effect of serum on YAP phosphorylation was transient as YAP phosphorylation was partially recovered 4 h after serum stimulation (FIG. 1A). Serum also caused a mobility shift of TAZ, suggesting TAZ was also dephosphorylated (FIG. 1A). Along with the decreased phosphorylation, protein levels of both YAP and TAZ, especially TAZ, were also increased by serum, consistent with previous observation that phosphorylation promotes YAP/TAZ degradation (Liu et al., 2010; Zhao et al., 2010b). The protein levels of upstream kinases Mst1 and Lats1 were largely unaffected under serum stimulation (FIG. 1A). The repression of YAP and TAZ phosphorylation by serum was confirmed in multiple cell lines including HeLa, RC3, SK-Mel-28, SF268, U205, and MCF10A. (FIG. 2A-D).

The effect of serum on YAP/TAZ phosphorylation was dose-dependent and rapid. YAP dephosphorylation was evident when as little as 0.5% serum was added (FIG. 1B). Moreover, removal of serum for as short as 15 min dramatically increased YAP/TAZ phosphorylation (FIG. 1C). Our data indicate that the effect of serum on YAP phosphorylation is likely a direct signaling event, as serum-induced YAP dephosphorylation was rapid (visible at 5 min, FIG. 2B) and reversible (FIG. 1C).

Phosphorylation of S127 in YAP leads to 14-3-3 binding and YAP cytoplasmic localization (Zhao et al., 2007). In consistence with that, we found that serum caused significant nuclear accumulation of YAP in both HEK293A and MCF10A cells (FIG. 1D). These data demonstrate that a component in serum could potently activate YAP by inducing dephosphorylation and nuclear localization.

### Identification of LPA as a YAP-Activating Component in Serum

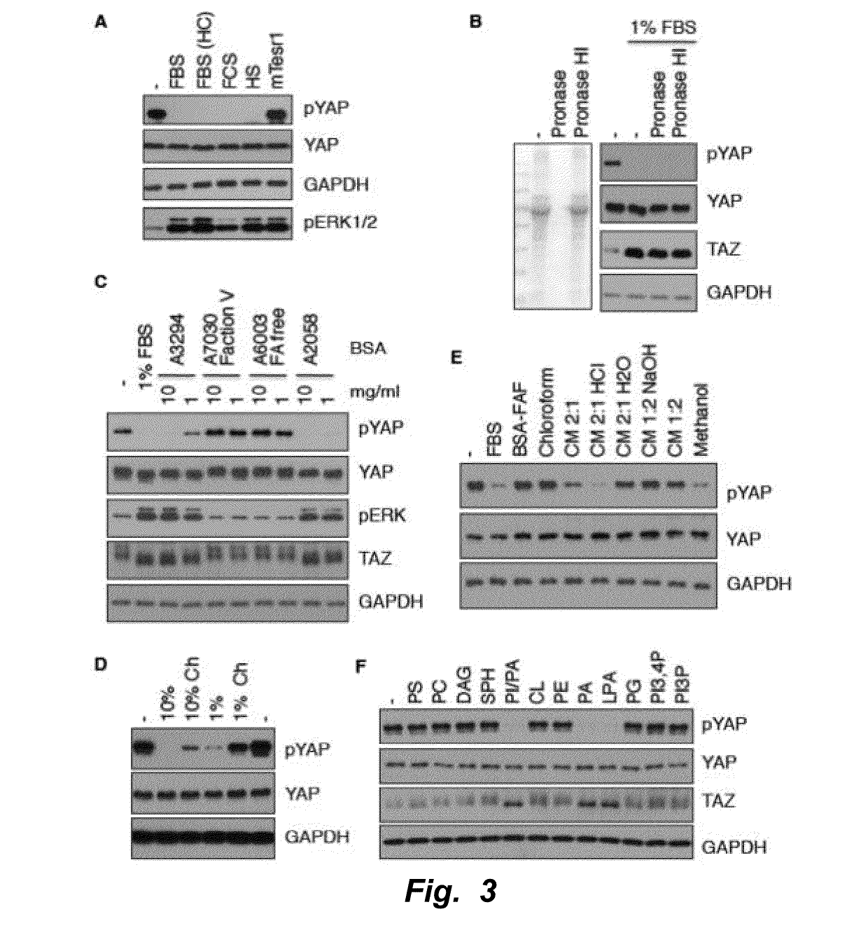

To rule out the possibility that the YAP/TAZ activating component(s) was present in a particular batch of serum, we examined serum from different sources and found they all could induce YAP dephosphorylation (FIG. 3A). However, embryonic stem cell culture medium (mTeSR1) that contains several growth factors showed no effect on YAP phosphorylation, although phosphorylation of extracellular-signal-regulated kinases (ERKs) was induced (FIG. 3A), suggesting that growth factors present in mTesR1 do not regulate YAP phosphorylation. Moreover, we tested several growth factors including insulin, EGF, FGF, and PDGF, and found that their evoked signaling pathways were not involved in YAP/TAZ activation (FIG. 2E, 2F), indicating that the active component(s) commonly present in serum is unlikely to be a general growth factor. Consistently, inhibition of MEK by U0126, PI3K by wortmannin, mTOR by torin, and p38 by SB253580 had no effect on the ability of FBS to dephosphorylate YAP/TAZ (FIG. 2G, 2H).

In order to determine if a protein component in serum is responsible for YAP/TAZ activation, we treated serum with pronase E which effectively degraded serum proteins. Interestingly, we found that the YAP/TAZ-dephosphorylating activity in serum was largely unaffected by pronase treatment (FIG. 3B). Moreover, the activity was resistant to heating and dialysis. These observations indicate that the YAP/TAZ-activating factor(s) in serum is likely not a protein molecule, and likely is a macromolecule or a small molecule tightly associated with a macromolecule.

Bovine serum albumin (BSA) was included as a control and we were surprised to find that BSA also potently decreased YAP/TAZ phosphorylation (FIG. 3C). BSA is a major serum component and is known to associate with different molecules. We thus tested different BSA preparations on YAP/TAZ phosphorylation. While some BSA preparations induced YAP/TAZ dephosphorylation, fatty acid-free BSA and Fraction V BSA displayed no activity towards YAP/TAZ phosphorylation (FIG. 3C). Similar to fatty acid-free BSA, the fraction V BSA, which likely has less lipid contamination because it was prepared by ethanol precipitation. These observations suggest that a hydrophobic compound in BSA, possibly a bioactive lipid, is responsible for inducing YAP/TAZ dephosphorylation. In support, charcoal-stripped FBS (with reduced lipid content) had a markedly lower ability to induce YAP dephosphorylation (FIG. 3D).

To further characterize the YAP/TAZ-dephosphorylating activity in FBS, we performed a series of extraction experiments using different organic solvents (Quehenberger et al., 2010). Chloroform was ineffective in extracting the activity whereas methanol or ethanol could extract the activity (FIG. 3E and data not shown). Moreover, a chloroform/methanol mixture could effectively extract the activity only at low pH but not at neutral or high pH (FIG. 3E). These results suggest that the active ingredient is an amphiphilic molecule with an acidic group. At low pH, the acidic group in the active component is not charged allowing it to be extracted by chloroform/methanol. In contrast, at neutral or high pH, the acidic group in the active component was charged, and thus could not partition into the organic solvents. Phospholipids and particularly lysophospholipids, which have hydrophobic tails with phosphate heads, represent the best-known groups of amphiphilic signaling molecules. Thus, we tested if phospholipids might induce YAP dephosphorylation. Among the phospholipids tested, we found that phosphatidic acid (PA), LPA, and a mixture of PA and phosphoinositol strongly induced dephosphorylation of YAP/TAZ (FIG. 3F).

### LPA and S1P Stimulate YAP/TAZ Activity

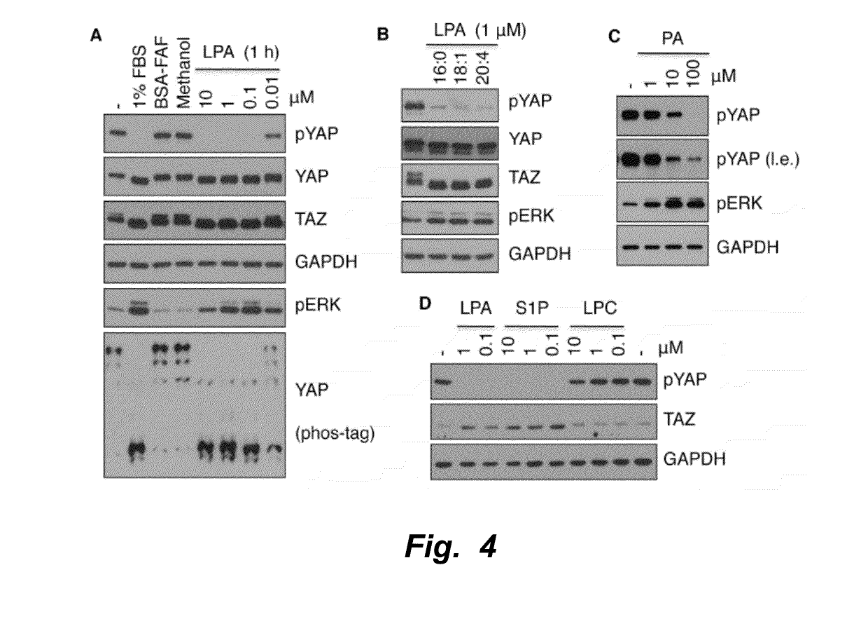

LPA is a family of glycerophospholipid signaling molecules present in all tissues and serum (Choi et al., 2009). Low concentrations of LPA were effective with 0.01 μM LPA inducing partial and 0.1 μM LPA inducing complete YAP/TAZ dephosphorylation (FIG. 4A), indicating LPA could activate YAP/TAZ at physiological (sub-micromolar) concentrations (Choi et al., 2009). Next, we examined various LPA isoforms with different lengths and degrees of saturation of the fatty acid tails and found that they all induced YAP/TAZ dephosphorylation (FIG. 4B). We subsequently tested PA and found that a much higher concentration, 100 μM, was needed to induce YAP dephosphorylation (FIG. 4C). Because PA can be converted to LPA by phospholipases, is 1,000 times less potent than LPA, and PA preparations are frequently contaminated with low levels of LPA, our data suggest that PA may not directly induce YAP dephosphorylation. Rather, the conversion of PA to LPA or residual LPA contamination in the PA preparation might contribute to the activity detected at high concentrations of PA (FIG. 3F).

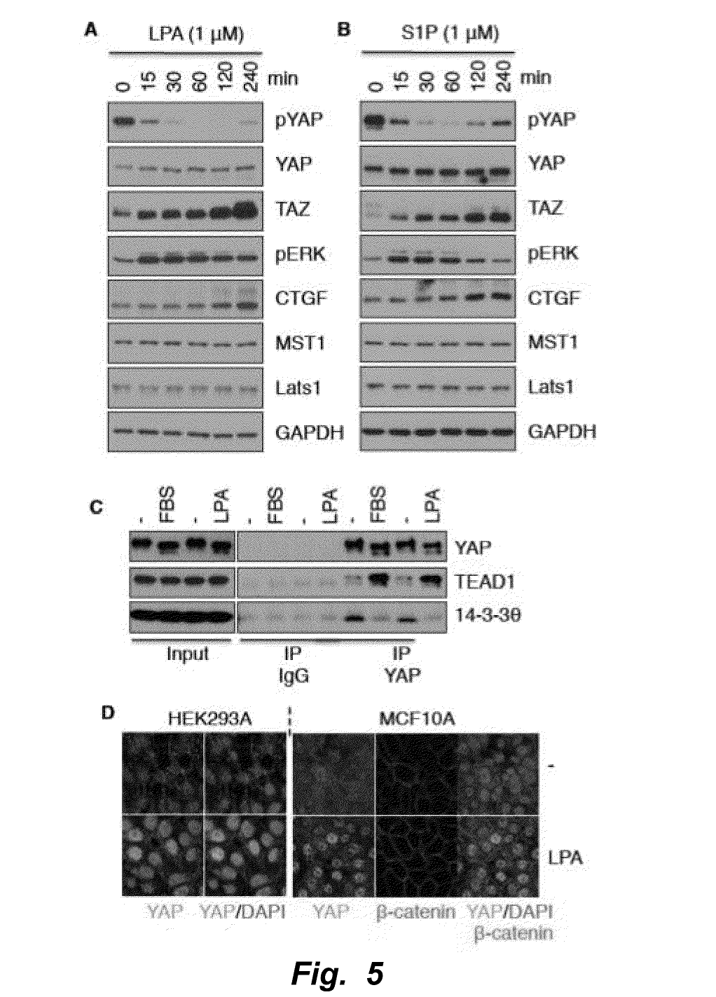

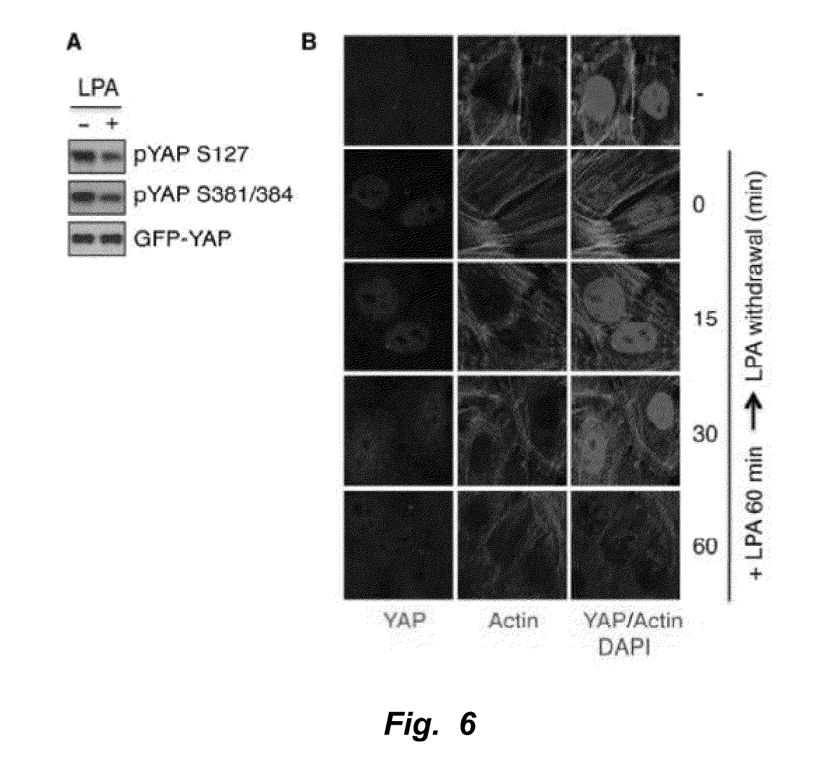

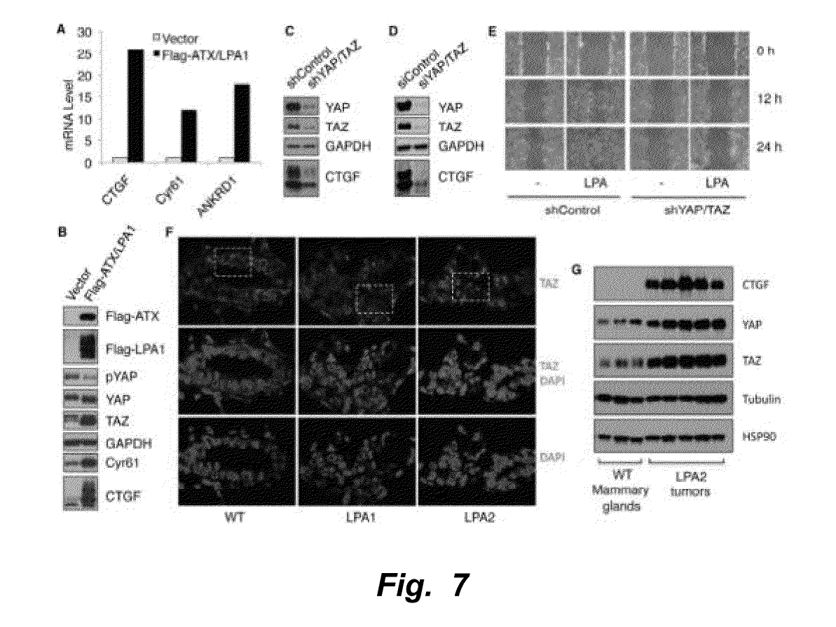

Similar to serum, LPA induced rapid YAP/TAZ dephosphorylation (FIG. 5A). As a result of YAP and TAZ activation, CTGF expression, which is a direct target gene of YAP/TAZ, was induced (FIG. 5A). We have previously shown that Lats can phosphorylate YAP on 5 serine residues including S381, and that phosphorylation at S381 primes S384 phosphorylation by casein kinase (Zhao et al., 2010b). Indeed, we found that phosphorylation of S381/384 was also decreased in response to LPA treatment (FIG. 6A).

The S1P group of lysophospholipids has similar physiological functions to LPA (Rosen et al., 2009). Therefore, the effect of S1P on YAP phosphorylation was investigated. Similar to LPA, S1P potently induced YAP/TAZ dephosphorylation (FIG. 5B, 4D). YAP phosphorylation is required for 14-3-3 binding and nuclear exclusion. Consistently, with its ability to promote YAP dephosphorylation, LPA treatment attenuated YAP-14-3-3 interaction (FIG. 5C) and induced YAP nuclear localization (FIG. 5D). The subcellular location of YAP was sensitive to LPA in a reversible manner, YAP protein redistributed into cytoplasm at 30 min after LPA withdrawal (FIG. 6B). LPA also enhanced the interaction between YAP and the nuclear-localized TEAD1, a transcription factor target of YAP/TAZ (FIG. 5C). Similar observations were made with S1P (data not shown). Taken together, these data demonstrate that LPA and S1P are novel activators of YAP/TAZ.

### YAP and TAZ are Involved in LPA to Stimulate Gene Expression, Cell Migration and Cell Proliferation

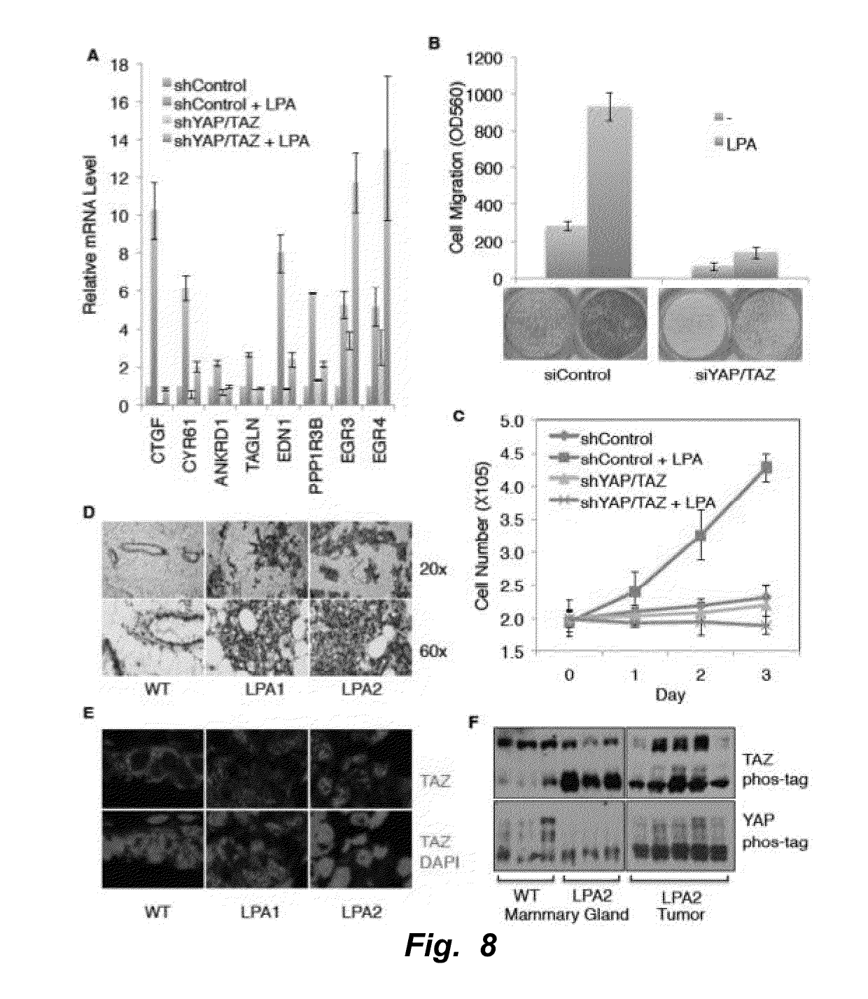

As a transcription co-activator, the major function of YAP/TAZ is to stimulate gene expression. CTGF, Cyr61 and ANKRD1 are well-characterized YAP target genes. Indeed, LPA, S1P and serum treatment induced the expression of CTGF (FIGS. 1A, 5A and 5B), and the mRNA and/or protein levels of CTGF, Cyr61 and ANKRD1 were also increased in cells stably expressing ectopic LPA1 and autotaxin (ATX, a LPA producing enzyme; FIGS. 7A and 7B). To determine the function of YAP/TAZ in LPA-induced gene expression, YAP and TAZ were knocked-down by shRNAs (FIG. 7C). We found that knockdown of YAP/TAZ strongly repressed the mRNA induction of CTGF, Cyr61, ANKRD1, TAGLN, EDN1, and PPP1R3B by LPA (FIG. 8A), supporting a role of YAP/TAZ in LAP-induced gene expression. However, LPA can also activate other transcription factors via different signaling pathways, such as ERK (FIG. 5A). Both EGR3 and ERG4 are LPA inducible genes that are regulated by ERK activation (Li et al., 2007; Ludwig et al., 2011). We examined these two genes and found that knockdown of YAP/TAZ did not block, but instead enhanced the expression of EGR3 and EGR4 (FIG. 8A). These data suggest that YAP may be involved in a feedback inhibition of LPA signaling. Nevertheless, our data supports a critical role for YAP/TAZ in LPA signaling required for the expression of some LPA inducible genes.

LPA is known to stimulate cell migration and has been implicated in tumor metastasis (Shida et al., 2003). We examined the effect of siRNA knockdown of YAP/TAZ in MCF10A cells (FIG. 7D) on cell migration using a transwell migration assay. LPA-stimulated cell migration was strongly inhibited in YAP/TAZ double knockdown cells (FIG. 8B). In a wound-healing assay, YAP/TAZ knockdown also blocked the effect of LPA on cell migration (FIG. 7E). Another well-characterized function of LPA is to promote cell proliferation (van Corven et al., 1989). HEK293A cells displayed little growth in the absence of serum; however, addition of LPA to serum free medium strongly induced cell proliferation. Interestingly, LPA failed to stimulate cell growth in the YAP/TAZ knockdown cells (FIG. 8C). Our data demonstrate an important role for YAP/TAZ in mediating the physiological functions of LPA in gene induction, cell migration, and proliferation.

It has been shown that elevated TAZ expression is associated in human breast cancer (Chan et al., 2008), and LPA receptor over-expression in mouse mammary gland causes hyperplasia and tumor formation (Liu et al., 2009). To determine the regulation of YAP/TAZ by LPA receptors in vivo, we analyzed an LPA receptor transgenic mouse model. As expected, LPA1 and LPA2 transgenic mammary tissues exhibited massive overgrowth (FIG. 8D). In contrast to cytoplasmic localization in control tissues, TAZ was enriched in cell nucleus of LPA1 and LPA2 transgenic tissues (FIGS. 8E and 7F). YAP/TAZ were dephosphorylated in LPA receptor transgenic mammary tissues and tumors (FIG. 8F). In LPA2 tumors, protein levels of YAP/TAZ and their target gene, CTGF, were significantly upregulated (FIG. 7G). The above observations support a role of LPA receptor in promoting YAP/TAZ dephosphorylation and activation in vivo.

### LPA Inhibits Lats1/2 Kinase Activity

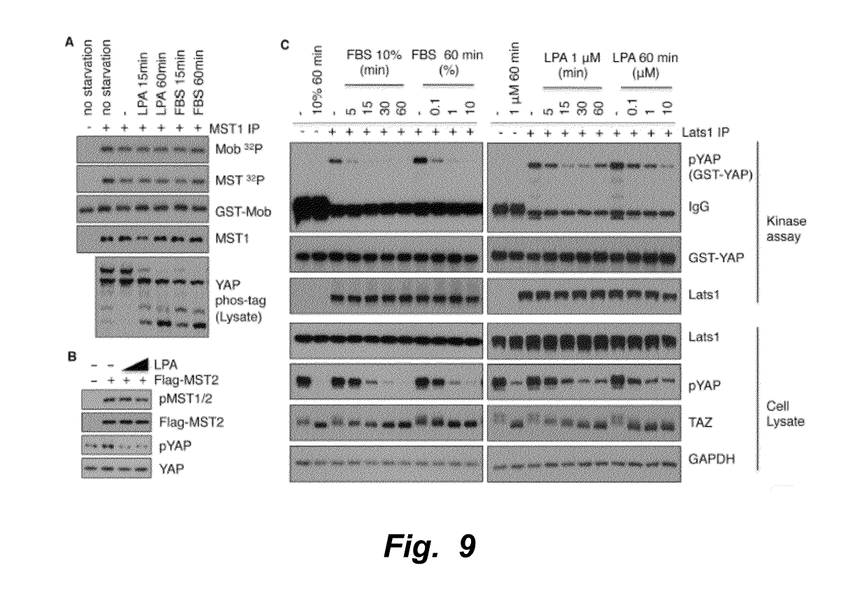

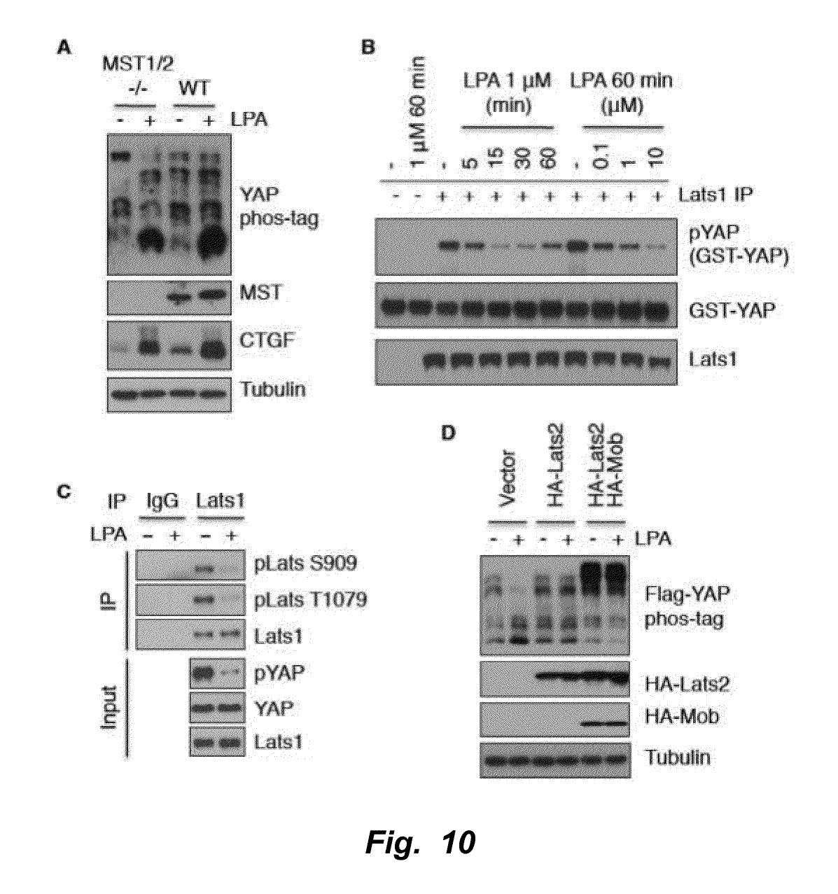

To determine whether LPA acts through the Hippo-YAP pathway kinases Mst1/2 and Lats1/2 to regulate YAP phosphorylation, we determined their kinase activity. We found that LPA and serum had no detectable effect on Mst1 kinase activity as visualized by Mob phosphorylation, which served as a MST substrate, and Mst1 autophosphorylation (FIG. 9A). The phosphorylation of MST2 at T180 was also not changed following LPA treatment (FIG. 9B). In addition, LPA could still induce YAP dephosphorylation in MST1/2 double knockout MEF cells (FIG. 10A), indicating that MST1/2 is not required for YAP regulation by LPA in MEF cells.

We also measured Lats1 kinase activity and found that Lats1 kinase activity was rapidly inhibited by serum or LPA treatment (FIG. 10B, 9C). The inhibition of Lats1 kinase activity by FBS and LPA correlated with the repression of endogenous YAP phosphorylation in both dose- and time-dependent manners (FIG. 9C), suggesting that LPA and serum decrease YAP phosphorylation by inhibiting Lats1/2 activity. Consistently with the observed Lats inhibition, phosphorylation levels of Lats1 at activation loop (S909) and hydrophobic motif (T1079), both correlating with Lats activity, were decreased upon LPA treatment (FIG. 10C). Moreover, in cells with Lats2 overexpression, the effect of LPA on YAP phosphorylation was abolished (FIG. 10D), again suggesting that inhibition of Lats1/2 kinase activity by LPA is required for LPA to decrease YAP phosphorylation. Our data show that LPA signaling acts upstream of Lats1/2 but parallel to Mst1/2.

### LPA/S1P Act Through GPCRs, G12/13, and Rho to Induce YAP/TAZ Dephosphorylation

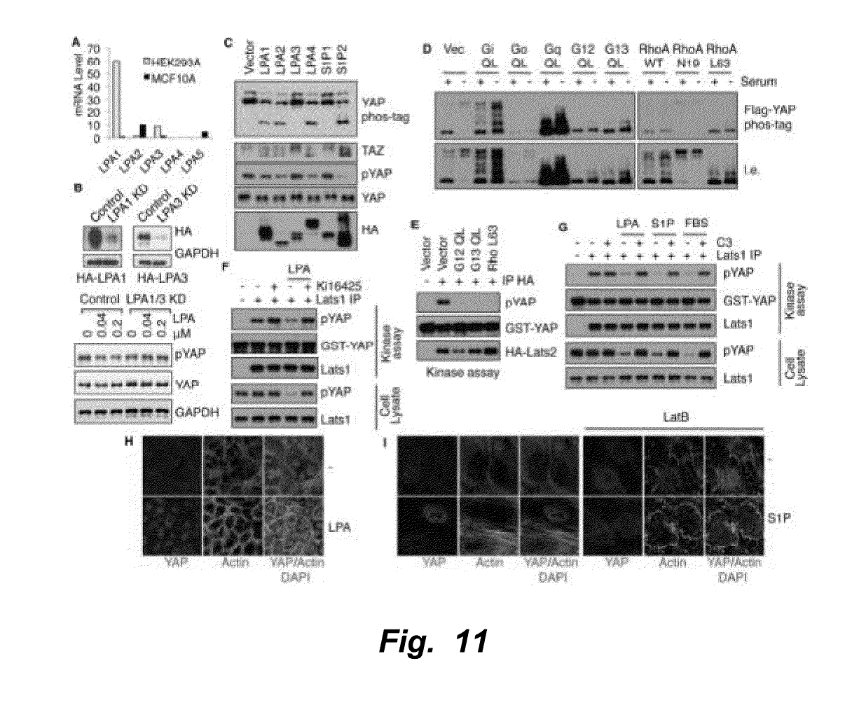

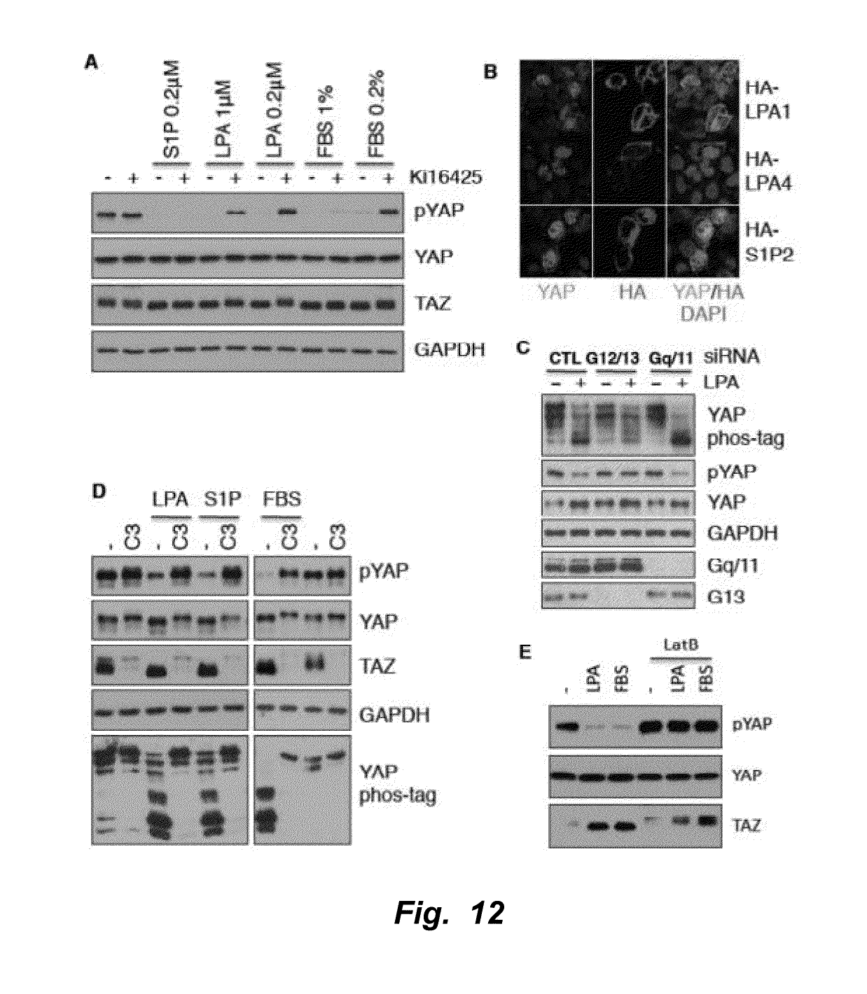

LPA binds to a family of GPCRs known as LPA receptors (LPA1-6) to initiate intracellular signaling (Choi et al., 2009). LPA1 was highly expressed and LPA3 was detectable in HEK293A cells compared to the other LPA receptors (FIG. 11A). To determine if LPA receptors were required for LPA-induced YAP/TAZ activation, we treated HEK293A cells with Ki16425, which preferentially inhibits LPA1 and LPA3 (Ohta et al., 2003). Ki16425 treatment blocked LPA- but not S1P-induced dephosphorylation of YAP/TAZ (FIG. 12A), suggesting LPA1 and LPA3 mediate LPA-induced YAP dephosphorylation in HEK293A cells. Consistently, the LPA-induced YAP dephosphorylation was significantly blocked by stable knockdown of LPA1 and LPA3 (FIG. 11B). Furthermore, ectopic expression of LPA and S1P receptors was sufficient to induce YAP nuclear localization and dephosphorylation (FIG. 12B, 12C). These data suggest that the function of LPA or S1P on Hippo-YAP pathway is mediated by their cognate transmembrane receptors. Notably, Ki16425 partially inhibited the ability of serum to repress YAP/TAZ phosphorylation, particularly at a low serum concentration (0.2%) (FIG. 12A). The Ki16425-insensitive YAP-dephosphorylating activity in serum could be due to S1P or other factors.

Both LPA and SIP receptors activate several heterotrimeric G proteins to initiate intracellular signaling pathways. To determine if Ga proteins are involved in YAP regulation, we tested the effect of Ga overexpression on YAP phosphorylation. Our data indicate that overexpression of wild type or active G12/13 could induce YAP dephosphorylation (FIG. 11D and Table 1). Indeed, knockdown of both G12 and G13 largely blocked LPA's effect on YAP dephosphorylation (FIG. 12C), suggesting that G12/13 play a major role in mediating LPA signaling to Hippo-YAP pathway.

TABLE 1
Over-expression of G alpha subunits modulate
YAP phosphorylation

| | | |
|---|---|---|
| G alpha | | |
| subunits | Mutation | p-YAP |

| | | | |
|---|---|---|---|
| G_{12/13} | G₁₂ | WT | ↓↓↓ |
| | G₁₂ | Q231L | ↓↓↓ |
| | G₁₃ | WT | ↓↓↓ |
| | G₁₃ | Q226L | ↓↓↓ |
| G₉ | G₉ | WT | — |
| | G₉ | Q209L | ↓↓↓ |
| | G₁₁ | WT | — |
| | G₁₁ | Q209L | ↓↓↓ |
| | G₁₄ | Q205L | ↓↓↓ |
| | G₁₅ | Q212L | ↓↓↓ |
| G_{i/o} | G₁₁ | Q204L | ↓ |
| | G₁₂ | Q205L | ↓ |
| | G₁₃ | Q204L | ↓ |
| | G₀ | Q205L | ↓ |
| | G₁₁ | Q200L | ↓ |
| | G₁₂ | Q204L | ↓ |
| | G₂ | Q205L | — |
| Gₛ | Gₛ | Q227L | ↑ |
| | G_{o/i} | Q214L | ↓ |

FLAG-WAP was co-transfected into HEK293A cells with different G alpha subunits. Following 24 h incubation in complete medium or serum-free medium, cell lysate were prepared and YAP phosphorylation status were assessed using phos-tag containing gels.
Arrows indicate up- or down- regulation of YAP phosphorylation.

Rho GTPases are known downstream mediators of G12/13 and LPA. We therefore expressed the RhoA-N19 dominant negative mutant and found that it blocked serum-induced YAP dephosphorylation (FIG. 11D). Conversely, expression of the constitutively active RhoA-L63 mutant induced a robust YAP dephosphorylation even in the absence of serum (FIG. 11D). In addition, botulinum toxin C3, a specific inhibitor of Rho GTPases, not only elevated the basal phosphorylation of YAP/TAZ but also blocked LPA-, S1P-, and serum-induced YAP/TAZ dephosphorylation (FIG. 12D). These data indicate an important role of Rho in mediating the LPA/S1P signal to YAP dephosphorylation. In support, co-transfection of active G12, G13 and RhoA repressed Lats2 kinase activity (FIG. 11E). Moreover, inhibition of LPA1 and LPA3 by Ki16425 and inactivation of Rho GTPases by C3 treatment effectively blocked Lats1 inhibition by LPA, S1P, and serum (FIG. 11F, 11G). Taken together, these data support a model wherein both LPA and S1P act through membrane GPCR, G12/13, and Rho GTPases to inhibit Lats1/2 activity and YAP phosphorylation.

The major function of Rho GTPases is to regulate cellular actin dynamics. A role of actin cytoskeleton on Hippo-YAP pathway has recently been suggested (Dupont et al., 2011; Fernandez et al., 2011; Rauskolb et al., 2011; Sansores-Garcia et al., 2011; Zhao et al., 2012), therefore we determined if actin cytoskeleton is important for YAP activation by LPA. The YAP nuclear localization under LPA or S1P treatment was correlated to levels of cellular actin filaments (FIG. S3B, S6H and S6I). When cells were treated with actin disrupting agents, latrunculin B (LatB), the effects of LPA or S1P on YAP were blocked (FIG. 12E, 11I). These results indicate that LPA or S1P may regulate Lats kinase activity by modulating actin cytoskeleton.

### Regulation of YAP Phosphorylation by GPCRs

GPCRs represent one of the largest gene families in the human genome. There are approximately 1,000 GPCRs that are coupled to fifteen different Ga proteins (Wettschureck and Offermanns, 2005). We asked whether other GPCRs, especially those that are not coupled to G12/13, could modulate YAP/TAZ activity. It is difficult to test the effect of many GPCR ligands because the expression of GPCRs is tissue specific and only a limited numbers of receptors are expressed in any given cell line. However, overexpression of GPCRs often can activate signaling as we observed by the overexpression of LPA receptors (FIGS. 12B and 11C). We therefore tested the effect of representative members of different GPCR subgroups on YAP/TAZ activity by overexpression. The results are summarized in Table 2. YAP/TAZ phosphorylation was modulated by many but not all GPCRs. A large number of G12/13-, or Gq/11-coupled receptors, such as purinergic receptor 1, platelet-activating factor receptor, thyroid stimulating hormone receptor and estrogen receptor 1 (GPER) could repress YAP/TAZ phosphorylation. On the other hand, GPCRs that mainly activate Gs signaling, such as β2 adrenergic receptor, dopamine receptor 1 and glucagon receptor, could induce YAP/TAZ phosphorylation (Table 2). These results indicate that different GPCRs can either stimulate or inhibit YAP/TAZ phosphorylation.

TABLE 2
Over-expression of GPCRs modulate YAP phosphorylation

| GPCR | G coupling | pYAP | pTAZ |
|---|---|---|---|
| Andrenergic receptor alpha 1B | Gq/11 | ↓ | ↑ |
| Lysophosphatidic acid receptor 5 (LPAR5) | G12/13, Gq/11 | ↓↓ | ↓↓ |
| Coagulation factor II (thrombin) receptor | G12/13, Gq/11, Gl/o | — | — |
| Endothelin receptor type A | Gq/11 | ↑ | ↑ |
| Purinergic receptor 9 (LPAR4) | G12/13, Gp/11, Gl/o, Gs | ↓↓↓ | ↓↓ |
| Chemokine (C—X—C motif) receptor 4 | G12/13, Gp/11, Gl/o | ↑ | ↑ |
| S-Hydroxytryptamine receptor 1A | Gl/o | — | ↓ |
| Muscarinic acetylcholine receptor M1 | Gq/11, Gl/o, Gs | ↓ | — |
| Adenosine receptor A1A | Gl/o | ↓↓ | ↑ |
| Adrenergic receptor beta 2 | Gs, Gl/o | — | ↑ |
| Angiotensin II receptor (variant 1) | Gq/11 | — | ↓ |
| Dopamine receptor D1 | Gs | ↑ | ↑ |
| Free Fatty Acid receptor 1 | Gq/11, Gl/o | ↓↓ | — |
| Histamine receptor 1 | Gq/11 | — | ↑ |
| Purinergic receptor 1 | Gq/11 | ↓↓↓ | ↓↓ |
| Platelet-activating factor receptor | Gq/11, Gl/o | ↓↓↓ | ↓ |
| Prostaglandin E receptor 1, subtype EP1 | Gq/11 | — | — |
| Thromboxine A2 | Gq/11 | ↓↓ | ↓ |
| Arginine vasopressin receptor 1A | Gq/11 | — | — |
| Frizzled homolog D4 (drosophila) | ? | ↓ | — |
| Glucagon receptor | Gl/o, Gs | ↑ | ↑ |
| Complement component 3a receptor 1 | Gl/o | ↓ | — |
| Estrogen receptor 1 | Gq/11, Gs? | ↓↓ | ↓↓ |
| Human glutamate receptor metabotropic 2 | Gl/o | ↓ | ? |
| Opioid receptor delta 1 | Gl/o | ↓ | ? |
| Secretin receptor | Gq/11, Gs | ↓↓ | ↓↓ |
| Thyroid stimulating hormone receptor | G12/13, Gq/11, Gl/o, Gs | ↓↓ | ↓ |
| Vasoactive intestinal peptide receptor 1 | Gs | — | — |
| Gastrin-releasing peptide receptor | Gq/11 | ↓↓ | ND |
| Thyrotroping-releasing hormone receptor | Gq/11 | — | ND |
| Melanocortin receptor 1 | Gs? | ↓↓ | ND |
| Somatostatin recepetor 1 | ? | ↓↓ | ND |
| Prostaglandin E receptor 2 | Gs | ↓↓ | ND |
| Bombesin like receptor 3 | Gq/11, G12/13 | ↓↓ | ND |

FLAG-YAP was co-transfected into HEK293A cells with different GPCRs. Following 24 h incubation in complete medium or serum-free medium, cell lysate were prepared and phosphorylation status of FLAG-YAP were assessed using phos-tag containing gels. Endogenous TAZ phosphorylation was also determined by immunoblotting.
Arrows indicate up- or down-regulation of YAP/TAZ phosphorylation.
Question mark denotes unsure information or inconsistent results.
ND indicates not determined.

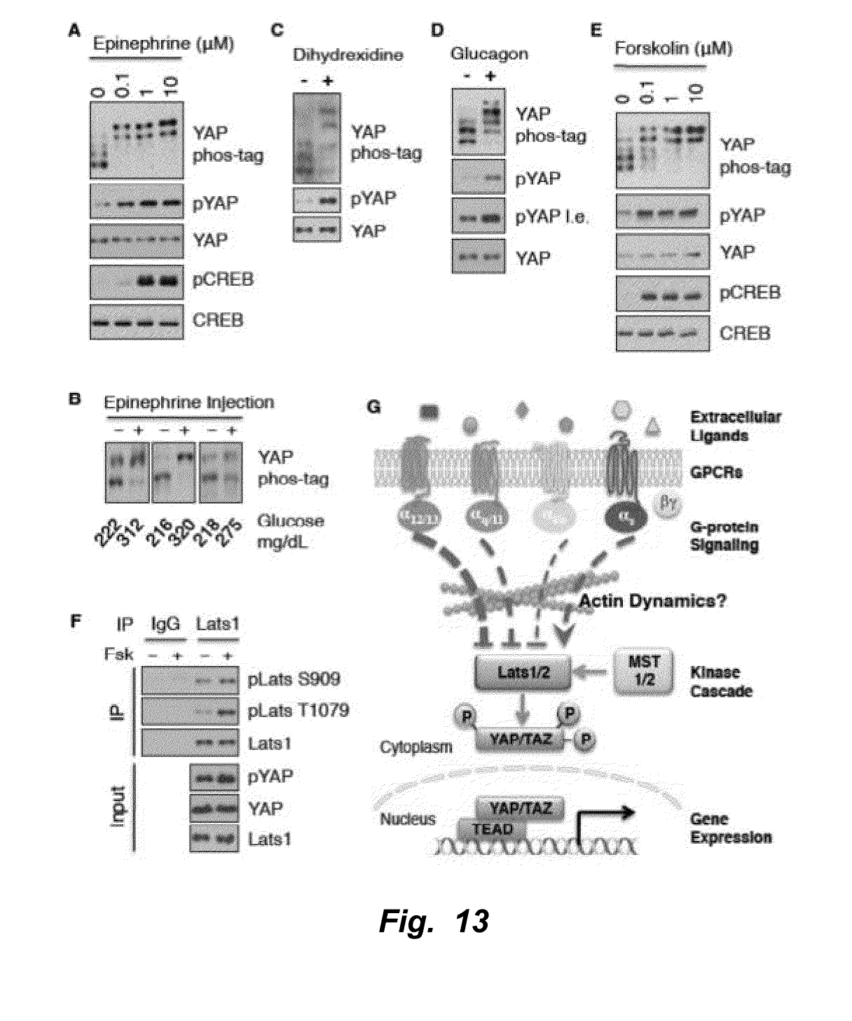

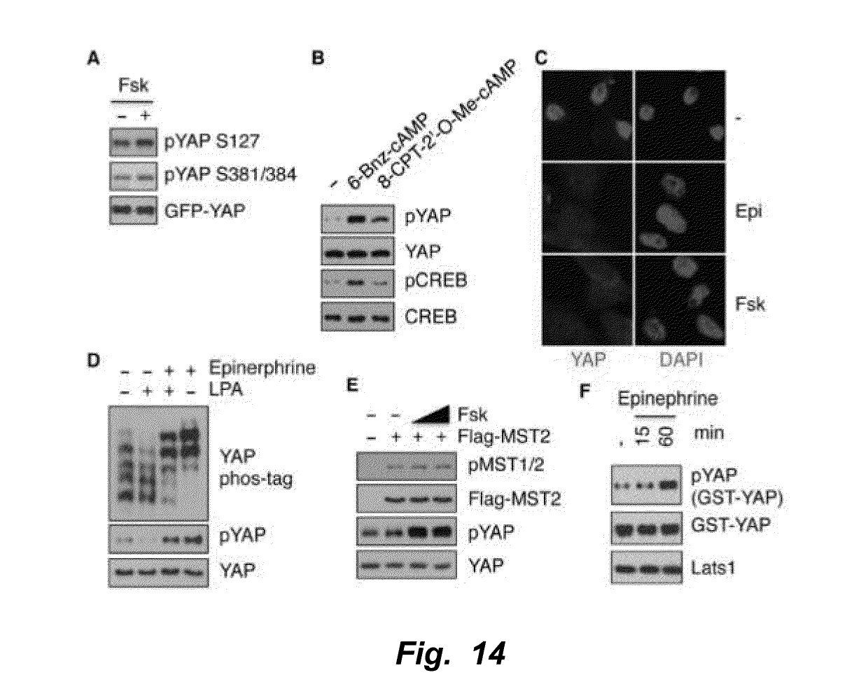

Overexpression of GPCRs could result in nonspecific activation of Ga that might not be activated under physiological conditions. To further establish the role of GPCRs in YAP regulation, we tested the effect of physiological hormones or GPCR agonists on YAP/TAZ phosphorylation using cell lines that are known to express their corresponding receptors. We were particularly interested in agonists that stimulate Gs-coupled receptors, as over-expression of Gs-coupled receptors induced YAP phosphorylation and their agonists may represent negative regulators for YAP/TAZ function. In MDA-MB-231 breast cancer cells, stimulation with epinephrine resulted in a dose-dependent phosphorylation of YAP (FIG. 13A). As expected, epinephrine increased phosphorylation of the cAMP responsive element binding protein (CREB), supporting that epinephrine indeed stimulated Gs and cAMP production. In addition, when fed mice were injected with epinephrine, YAP phosphorylation was significantly increased in heart, a physiological targeting organ of epinephrine (FIG. 13B), suggesting a role of epinephrine in YAP regulation in vivo.

Overexpression of dopamine receptor 1 or glucagon receptor, which are known to activate Gs, also increased YAP phosphorylation (Table 2). To extend the notion that activation of Gs by other GPCR agonists also increases YAP phosphorylation, we examined the effect of dihydrexidine, an agonist for dopamine receptor 1 and 5. Dihydrexidine treatment caused a strong increase of YAP phosphorylation in U2OS cells (FIG. 13C). Glucagon receptor is expressed in hepatocytes. We thus isolated primary mouse hepatocytes and tested the effect of glucagon. As shown in FIG. 13D, glucagon treatment increased YAP phosphorylation as determined by both mobility shift and immunoblotting with the phospho-YAP antibody. The above data support a conclusion that activation of Gs coupled receptors results in YAP hyperphosphorylation and inactivation under physiological conditions.

To further confirm the role of cAMP signaling in the Hippo-YAP pathway, we treated cells with Forskolin, an activator of adenylyl cyclase producing cAMP, and found that it also increased YAP phosphorylation (FIG. 13E). The cAMP signaling cascade can activate protein kinase A (PKA) or exchange protein activated by cAMP (Epac). We found that the PKA selective activator, b-Bnz-cAMP, dramatically increased YAP phosphorylation; whereas, the effect of an Epac selective activator, 8-CPT-2′-O-Me-cAMP, on YAP phosphorylation was less dramatic (FIG. 14B). Therefore, Gs coupled GPCR can induce YAP phosphorylation mainly via cAMP and PKA.

Consistent with the increase of YAP phosphorylation, immunofluorescence staining with YAP-specific antibody demonstrated an accumulation of cytoplasmic YAP under epinephrine and Forskolin treatments (FIG. 14C). We determined whether Gs coupled signals worked in an opposite way as G12/13 and Gq/11 coupled signals, and indeed, epinephrine and LPA antagonized each other's effect on YAP phosphorylation (FIG. 14D). We also observed that Forskolin increased Lats1 phosphorylation, but not MST (FIG. 13F, 14E). Moreover, epinephrine increased Lats1 activity (FIG. 14F). Our data suggests that Gs-initiated signaling stimulates Lats kinase activity, therefore inhibits YAP/TAZ by phosphorylation.

### Differential Functions of Gα in Regulation of YAP Phosphorylation

Finally, we tested all Gα subunits for their ability to modulate YAP phosphorylation by overexpression (Table 1). Because only the GTP-bound Gα is active and directly participated in signaling, we expressed constitutively active mutants (GTP-bound form) of all Ga. We found that active Gα mutants decreased YAP phosphorylation at varying degrees with the exception of Gs and Gz. Among the Gα that decreased YAP phosphorylation, G12, G13, Gq, G11, G14, G15 were more potent than Gi, Gt, and Go in repressing YAP phosphorylation. Moreover, expression of the wild type G12 or G13 but not the wild type G11 or Gq was sufficient to inhibit YAP phosphorylation. These results indicate that G12/13 may be the most potent inhibitor of the Hippo-YAP pathway followed by Gq, G11, G14, G15 (these four belong to Gq/11 subfamily), whereas the effect of Gi, Gt, and Go (all belong to Gi/o subfamily) is less potent. On the other hand, expression of the constitutive active Gs mutant increased YAP phosphorylation. Together, these data further support differential roles of various Gα, hence GPCRs and their corresponding ligands in Hippo-YAP regulation (FIG. 13G).

### Discussion

The Hippo-YAP signaling pathway has been shown to regulate organ size and tumorigenesis (reviewed in Zhao et al., 2010a). However, the upstream signals remain elusive. Several studies have implicated that cell contact and mechanic force affect YAP phosphorylation whereas the exact nature of the signals and receptors are unknown (Dupont et al., 2011; Fernandez et al., 2011; Nishioka et al., 2009; Rauskolb et al., 2011; Sansores-Garcia et al., 2011; Wada et al., 2011; Zhao et al., 2012; Zhao et al., 2007). In this report, we have demonstrated that numerous extracellular signaling molecules act through cell surface GPCRs to modulate Lats1/2 kinases, thereby controlling YAP/TAZ phosphorylation and activity. To our knowledge, this is the first demonstration of physiological extracellular signaling molecules and transmembrane receptors in regulation of the Hippo-YAP pathway. Our study also establishes the Hippo-YAP pathway as an important signaling branch downstream of many GPCRs, thus expanding the scope of GPCR signaling.

### Regulation of the Hippo-YAP Pathway by a Wide Range of Extracellular Cues

Genetic studies have defined the core components of the Hippo-YAP pathway including Mst1/2, Say, Lats1/2, Mob, and YAP. Subsequent extensive genetic and biochemical investigations led to the identification of many proteins that modulate the Hippo-YAP pathway. These include Merlin, Angiomotin, α-catenin, Scribble, Ajuba, and RASSF (reviewed in Genevet and Tapon, 2011; Zhao et al., 2010a). Although a potential role of CD44 on Hippo-YAP pathway has been suggested (Xu et al., 2010), further studies are needed to demonstrate the physiological relevance of CD44 in Hippo-YAP pathway regulation. Therefore, a key missing issue in Hippo-YAP pathway is the identity of its extracellular signals and cell surface receptors.

Here, we demonstrate that serum contains activity to inhibit YAP/TAZ phosphorylation. Based on extensive biochemical characterizations, we identified LPA and S1P as potent serum-borne signals for the Hippo-YAP pathway. In addition, we have discovered that epinephrine, glucagon and dihydrexidine can stimulate YAP phosphorylation. These findings suggest that the Hippo-YAP pathway can be both positively and negatively regulated by diverse extracellular signals.

Our results indicate that the activity of Lats1/2 is modulated by these extracellular signals. In contrast, Mst1/2 kinases are not required in the LPA-induced Lats1/2 inhibition. Although this mechanism is rather intriguing, it is not totally surprising as Mst1/2 independent phosphorylation of Lats and YAP have been reported in the Mst1/2 knockout MEF cells (Zhou et al., 2009). The Rho GTPase acts downstream of LPA receptors and upstream of the Lats1/2 kinase. Rho may regulate Lats active via modulation of actin cytoskeleton.

The effect of serum, LPA and S1P on YAP/TAZ phosphorylation is transient, however this transient dephosphorylation is sufficient to induce YAP nuclear localization. Moreover, the transient YAP nuclear localization can induce gene expression, which may generate a long-term physiological effect, such as cell migration and proliferation. Transient activation of downstream signaling events is common to most signal transduction pathways. For example, ERK1/2 activation by EGF, LPA, and serum are transient. Actually, ERK activity reaches maximum at 5 minutes and returns to almost basal level at 30 minutes upon EGF stimulation, a time course much faster than YAP activation by LPA. Nevertheless, the transient ERK activation is sufficient to induce sustained effects, such as gene expression and proliferation. Most initial signaling events induced by GPCR are transient. A variety of mechanisms are in place to ensure the transient nature of GPCR signaling, such as G-protein-coupled receptor kinases terminating GPCR signaling or phosphodiesterases hydrolyzing cAMP.

### Hippo-YAP Pathway as a Signal Mediator of GPCR

All signals that modulate Hippo-YAP identified in this study turn out to be agonists for GPCRs. GPCR signaling regulates a wide array of physiological functions and represents the major targets for therapeutic drugs. Our study places Hippo-YAP pathway as a downstream signaling branch of GPCR. We propose that Lats1/2 kinases are inhibited by G12/13, Gq/11 and Gi/o coupled receptors and activated by Gs coupled receptors (FIG. 13G). Depending on the nature of receptors expressed and their coupled G-proteins, the activity of YAP and TAZ can be either stimulated or inhibited by GPCR agonists.

YAP and TAZ are transcription co-activators, therefore their activation/inhibition may play an important role in gene expression in response to GPCR activation. Consistent with this model, YAP/TAZ is required for the expression of some LPA-induced genes, indicating a direct role of YAP/TAZ in the transcriptional response of GPCR. YAP/TAZ plays a critical role in cell proliferation and cell migration in response to LPA. These results suggest that the Hippo-YAP pathway could mediate physiological functions of GPCR signaling.

GPCR activation has been linked to cell proliferation, and many mechanisms have been proposed (Dorsam and Gutkind, 2007). Gq/11, G12/13 and Gi/o coupled receptors usually show stimulatory effect on cell proliferation. This is consistent with their function on YAP/TAZ activation. The role of Gs coupled receptors in cell proliferation is rather complex although activation of Gs and PKA is generally associated with growth inhibition (Stork and Schmitt, 2002) Inhibition of YAP/TAZ activity by Gs coupled receptor signaling may lead to growth inhibitory on some types of cells. We noticed that the basal YAP/TAZ activity varies significantly in different cell lines, YAP/TAZ may not respond to Gs-coupled signaling when basal activity is low (highly phosphorylated), and an alternative signaling may promote cell proliferation.

### Complexity of Hippo-YAP Regulation

The regulation of Hippo-YAP pathway by multiple signals is not surprising given the important role of this pathway in cell proliferation and apoptosis, hence organ size control, and tumorigenesis. Multiple regulators may coordinate with each other to fine-tune physiological and pathological processes. This scenario is similar to MAP kinases or PI3 kinases, which are regulated by a large numbers of hormones via RTKs and other receptors. It is worth noting that YAP phosphorylation is not affected by the RTK ligands tested (FIG. 4).

Our results suggest that the upstream signals for Hippo-YAP pathway is highly redundant (FIG. 13G). GPCRs represent the largest class of cell surface receptors, and many GPCRs can be coupled to G12/13, Gq/11, or Gi/o. It is likely that many hormones acting through these G-proteins will similarly inhibit Lats1/2 kinase activity and decrease YAP phosphorylation. Conversely, hormones that activate Gs coupled GPCRs should increase Lats1/2 kinase activity and YAP phosphorylation.

Regulation of Hippo-YAP by GPCR can be rather complex due to the presence of multiple receptors for a single agonist. For example, LPA has at least 6 receptors, which can be coupled to different G-proteins. Therefore, it is possible that one ligand may increase YAP phosphorylation in one cell type but decrease YAP phosphorylation in another cell type depending on which receptor is dominantly expressed and which Ga is coupled to the receptor in the particular cell. We reason that the high redundancy and complexity may hinder genetic efforts to isolate upstream signals and receptors for the Hippo-YAP pathway, knockout or knockdown of a single GPCR may not significantly affect the Hippo-YAP pathway.

### Implication of GPCR-YAP Signaling in Organ Size and Cancer

Organ size control is a fundamental issue in biology and the final organ size is determined both intrinsically and extrinsically. The identification of GPCR ligands as Hippo-YAP pathway regulators opens new possibility to the role of the Hippo-YAP pathway in organ size control. It is possible that certain GPCR activating hormones play central role in organ size control through the Hippo-YAP pathway. It is also possible that organ specific GPCR ligands act as tissue specific negative growth regulators to restrict size of specific organs. Indeed, it has been shown that knockout of gprc6a in Leydig cells reduce testis size (Oury et al., 2011). Gprc6a is able to activate Gq (Kuang et al., 2005; Wellendorph et al., 2005), and it is possible that YAP activity is compromised in gprc6a knockout cells and contributes to the small organ size phenotype. The Hippo-YAP pathway also plays a role in nervous system (Cao et al., 2008). The effect of dopamine receptor agonist on YAP activity demonstrated in this study also indicates that Hippo-YAP pathway could be dynamically regulated by neurotransmitters. Therefore, it is also possible that a neuroendocrine mechanism is involved in organ size control.

Elevated YAP/TAZ nuclear localization is observed in many types of human cancers (Overholtzer et al., 2006; Steinhardt et al., 2008; Zender et al., 2006; Zhao et al., 2007), however the mechanism behind YAP/TAZ activation in cancer is largely unknown. The connection between GPCR and the Hippo-YAP pathway revealed by this study may provide a possible explanation for YAP/TAZ activation in certain tumors. GPCR signaling plays potent roles in cancer development (Dorsam and Gutkind, 2007). We have demonstrated here that transgenic expression of LPA receptors induced YAP/TAZ activity and that the oncogenic activity of YAP/TAZ may contribute to the hyperplasia and tumor phenotype in these mice. Moreover, thrombin receptor transgenic mice also exhibited hyperplasia phenotype (Frateschi et al., 2011), similar to YAP transgenic mice (Schlegelmilch et al., 2011). Some GPCRs (Stephens et al., 1993) and G-proteins (active G12) have transforming ability when over-expressed. Activating mutations of Gq and G11 are frequently associated with uveal melanoma, the most common tumor in the eye (Van Raamsdonk et al., 2010). In fact, approximately 83% of uveal melanoma has activating mutations in either Gq or G11 in a mutually exclusive manner. Based on our study, one may predict that constitutive activation of Gq or G11 in uveal melanomas results in abnormal YAP activation, which then contributes to uveal melanoma development. Moreover, familial and somatic activating mutations of GPCRs have been also been linked to human cancer (Dorsam and Gutkind, 2007).

### REFERENCES

Benhamouche, S., Curto, M., Saotome, I., Gladden, A. B., Liu, C. H., Giovannini, M., and McClatchey, A. I. (2010). Nf2/Merlin controls progenitor homeostasis and tumorigenesis in the liver. Genes Dev 24, 1718-1730.
Cai, J., Zhang, N., Zheng, Y., de Wilde, R. F., Maitra, A., and Pan, D. (2010). The Hippo-YAP signaling pathway restricts the oncogenic potential of an intestinal regeneration program. Genes Dev 24, 2383-2388.
Camargo, F. D., Gokhale, S., Johnnidis, J. B., Fu, D., Bell, G. W., Jaenisch, R., and Brummelkamp, T. R. (2007). YAP1 increases organ size and expands undifferentiated progenitor cells. Curr Biol 17, 2054-2060.
Cao, X., Pfaff, S. L., and Gage, F. H. (2008). YAP regulates neural progenitor cell number via the TEA domain transcription factor. Genes Dev 22, 3320-3334.
Chan, S. W., Lim, C. J., Guo, K., Ng, C. P., Lee, I., Hunziker, W., Zeng, Q., and Hong, W. (2008). A role for TAZ in migration, invasion, and tumorigenesis of breast cancer cells. Cancer Res 68, 2592-2598.
Choi, J. W., Herr, D. R., Noguchi, K., Yung, Y. C., Lee, C. W., Mutoh, T., Lin, M. E., Teo, S. T., Park, K. E., Mosley, A. N., et al. (2009). LPA receptors: subtypes and biological actions. Annu Rev Pharmacol Toxicol 50, 157-186.
Dong, J., Feldmann, G., Huang, J., Wu, S., Zhang, N., Comerford, S. A., Gayyed, M. F., Anders, R. A., Maitra, A., and Pan, D. (2007). Elucidation of a universal size-control mechanism in Drosophila and mammals. Cell 130, 1120-1133.
Dorsam, R. T., and Gutkind, J. S. (2007). G-protein-coupled receptors and cancer. Nat Rev Cancer 7, 79-94.
Dupont, S., Morsut, L., Aragona, M., Enzo, E., Giulitti, S., Cordenonsi, M., Zanconato, F., Le Digabel, J., Forcato, M., Bicciato, S., et al. (2011). Role of YAP/TAZ in mechanotransduction. Nature 474, 179-183.
Fernandez, B. G., Gaspar, P., Bras-Pereira, C., Jezowska, B., Rebelo, S. R., and Janody, F. (2011). Actin-Capping Protein and the Hippo-YAP pathway regulate F-actin and tissue growth in Drosophila. Development 138, 2337-2346.
Frateschi, S., Camerer, E., Crisante, G., Rieser, S., Membrez, M., Charles, R. P., Beermann, F., Stehle, J. C., Breiden, B., Sandhoff, K., et al. (2011). PAR2 absence completely rescues inflammation and ichthyosis caused by altered CAP1/Prss8 expression in mouse skin. Nat Commun 2, 161.
Genevet, A., and Tapon, N. (2011). The Hippo-YAP pathway and apico-basal cell polarity. Biochem J 436, 213-224.
Hamaratoglu, F., Willecke, M., Kango-Singh, M., Nolo, R., Hyun, E., Tao, C., Jafar-Nejad, H., and Halder, G. (2006). The tumour-suppressor genes NF2/Merlin and Expanded act through Hippo signalling to regulate cell proliferation and apoptosis. Nat Cell Biol 8, 27-36.
Hao, Y., Chun, A., Cheung, K., Rashidi, B., and Yang, X. (2008). Tumor suppressor LATS1 is a negative regulator of oncogene YAP. J Biol Chem 283, 5496-5509.
Heallen, T., Zhang, M., Wang, J., Bonilla-Claudio, M., Klysik, E., Johnson, R. L., and Martin, J. F. (2011). Hippo-YAP pathway inhibits Wnt signaling to restrain cardiomyocyte proliferation and heart size. Science 332, 458-461.
Kuang, D., Yao, Y., Lam, J., Tsushima, R. G., and Hampson, D. R. (2005). Cloning and characterization of a family C orphan G-protein coupled receptor. J Neurochem 93, 383-391.
Lee, C. H., Inoki, K., and Guan, K. L. (2007). mTOR pathway as a target in tissue hypertrophy. Annu Rev Pharmacol Toxicol 47, 443-467.
Lee, K. P., Lee, J. H., Kim, T. S., Kim, T. H., Park, H. D., Byun, J. S., Kim, M. C., Jeong, W. I., Calvisi, D. F., Kim, J. M., et al. (2010). The Hippo-Salvador pathway restrains hepatic oval cell proliferation, liver size, and liver tumorigenesis. Proc Natl Acad Sci USA 107, 8248-8253.
Lei, Q. Y., Zhang, H., Zhao, B., Zha, Z. Y., Bai, F., Pei, X. H., Zhao, S., Xiong, Y., and Guan, K. L. (2008). TAZ promotes cell proliferation and epithelial-mesenchymal transition and is inhibited by the Hippo-YAP pathway. Mol Cell Biol 28, 2426-2436.
Li, L., Yun, S. H., Keblesh, J., Trommer, B. L., Xiong, H., Radulovic, J., and Tourtellotte, W. G. (2007). Egr3, a synaptic activity regulated transcription factor that is essential for learning and memory. Mol Cell Neurosci 35, 76-88.
Liu, C. Y., Zha, Z. Y., Zhou, X., Zhang, H., Huang, W., Zhao, D., Li, T., Chan, S. W., Lim, C. J., Hong, W., et al. (2010). The hippo tumor pathway promotes TAZ degradation by phosphorylating a phosphodegron and recruiting the SCF{beta}-TrCP E3 ligase. J Biol Chem 285, 37159-37169.
Liu, S., Umezu-Goto, M., Murph, M., Lu, Y., Liu, W., Zhang, F., Yu, S., Stephens, L. C., Cui, X., Murrow, G., et al. (2009). Expression of autotaxin and lysophosphatidic acid receptors increases mammary tumorigenesis, invasion, and metastases. Cancer Cell 15, 539-550.
Lu, L., Li, Y., Kim, S. M., Bossuyt, W., Liu, P., Qiu, Q., Wang, Y., Halder, G., Finegold, M. J., Lee, J. S., et al. (2010). Hippo signaling is a potent in vivo growth and tumor suppressor pathway in the mammalian liver. Proc Natl Acad Sci USA 107, 1437-1442.
Ludwig, A., Uvarov, P., Soni, S., Thomas-Crusells, J., Airaksinen, M. S., and Rivera, C. (2011). Early growth response 4 mediates BDNF induction of potassium chloride cotransporter 2 transcription. J Neurosci 31, 644-649.
Nishioka, N., Inoue, K., Adachi, K., Kiyonari, H., Ota, M., Ralston, A., Yabuta, N., Hirahara, S., Stephenson, R. O., Ogonuki, N., et al. (2009). The Hippo-YAP signaling pathway components Lats and Yap pattern Tead4 activity to distinguish mouse trophectoderm from inner cell mass. Dev Cell 16, 398-410.
Ohta, H., Sato, K., Murata, N., Damirin, A., Malchinkhuu, E., Kon, J., Kimura, T., Tobo, M., Yamazaki, Y., Watanabe, T., et al. (2003). Ki16425, a subtype-selective antagonist for EDG-family lysophosphatidic acid receptors. Mol Pharmacol 64, 994-1005.
Oury, F., Sumara, G., Sumara, O., Ferron, M., Chang, H., Smith, C. E., Hermo, L., Suarez, S., Roth, B. L., Ducy, P., et al. (2011). Endocrine regulation of male fertility by the skeleton. Cell 144, 796-809.
Overholtzer, M., Zhang, J., Smolen, G. A., Muir, B., Li, W., Sgroi, D. C., Deng, C. X., Brugge, J. S., and Haber, D. A. (2006). Transforming properties of YAP, a candidate oncogene on the chromosome 11q22 amplicon. Proc Natl Acad Sci USA 103, 12405-12410.
Pan, D. (2007). Hippo signaling in organ size control. Genes Dev 21, 886-897.
Quehenberger, O., Armando, A. M., Brown, A. H., Milne, S. B., Myers, D. S., Merrill, A. H., Bandyopadhyay, S., Jones, K. N., Kelly, S., Shaner, R. L., et al. (2010). Lipidomics reveals a remarkable diversity of lipids in human plasma. J Lipid Res 51, 3299-3305.
Rauskolb, C., Pan, G., Reddy, B. V., Oh, H., and Irvine, K. D. (2011). Zyxin links fat signaling to the Hippo-YAP pathway. PLoS Biol 9, e1000624.
Rosen, H., Gonzalez-Cabrera, P. J., Sanna, M. G., and Brown, S. (2009). Sphingosine 1-phosphate receptor signaling. Annu Rev Biochem 78, 743-768.
Sansores-Garcia, L., Bossuyt, W., Wada, K., Yonemura, S., Tao, C., Sasaki, H., and Halder, G. (2011). Modulating F-actin organization induces organ growth by affecting the Hippo-YAP pathway. EMBO J 30, 2325-2335.
Schlegelmilch, K., Mohseni, M., Kirak, O., Pruszak, J., Rodriguez, J. R., Zhou, D., Kreger, B. T., Vasioukhin, V., Avruch, J., Brummelkamp, T. R., et al. (2011). Yapl acts downstream of alpha-catenin to control epidermal proliferation. Cell 144, 782-795.
Shida, D., Kitayama, J., Yamaguchi, H., Okaji, Y., Tsuno, N. H., Watanabe, T., Takuwa, Y., and Nagawa, H. (2003). Lysophosphatidic acid (LPA) enhances the metastatic potential of human colon carcinoma DLD1 cells through LPA1. Cancer Res 63, 1706-1711.
Song, H., Mak, K. K., Topol, L., Yun, K., Hu, J., Garrett, L., Chen, Y., Park, O., Chang, J., Simpson, R. M., et al. (2010). Mammalian Mst1 and Mst2 kinases play essential roles in organ size control and tumor suppression. Proc Natl Acad Sci USA 107, 1431-1436.
Steinhardt, A. A., Gayyed, M. F., Klein, A. P., Dong, J., Maitra, A., Pan, D., Montgomery, E. A., and Anders, R. A. (2008). Expression of Yes-associated protein in common solid tumors. Hum Pathol 39, 1582-1589.
Stephens, E. V., Kalinec, G., Brann, M. R., and Gutkind, J. S. (1993). Transforming G protein-coupled receptors transduce potent mitogenic signals in NIH 3T3 cells independent on cAMP inhibition or conventional protein kinase C. Oncogene 8, 19-26.
Stork, P. J., and Schmitt, J. M. (2002). Crosstalk between cAMP and MAP kinase signaling in the regulation of cell proliferation. Trends Cell Biol 12, 258-266.
van Corven, E. J., Groenink, A., Jalink, K., Eichholtz, T., and Moolenaar, W. H. (1989). Lysophosphatidate-induced cell proliferation: identification and dissection of signaling pathways mediated by G proteins. Cell 59, 45-54.
Van Raamsdonk, C. D., Griewank, K. G., Crosby, M. B., Garrido, M. C., Vemula, S., Wiesner, T., Obenauf, A. C., Wackernagel, W., Green, G., Bouvier, N., et al. (2010). Mutations in GNA11 in uveal melanoma. N Engl J Med 363, 2191-2199.
Wada, K., Itoga, K., Okano, T., Yonemura, S., and Sasaki, H. (2011). Hippo-YAP pathway regulation by cell morphology and stress fibers. Development 138, 3907-3914.
Wellendorph, P., Hansen, K. B., Balsgaard, A., Greenwood, J. R., Egebjerg, J., and Brauner-Osborne, H. (2005). Deorphanization of GPRC6A: a promiscuous L-alpha-amino acid receptor with preference for basic amino acids. Mol Pharmacol 67, 589-597.
Wettschureck, N., and Offermanns, S. (2005). Mammalian G proteins and their cell type specific functions. Physiol Rev 85, 1159-1204.
Xin, M., Kim, Y., Sutherland, L. B., Qi, X., McAnally, J., Schwartz, R. J., Richardson, J. A., Bassel-Duby, R., and Olson, E. N. (2011). Regulation of insulin-like growth factor signaling by Yap governs cardiomyocyte proliferation and embryonic heart size. Sci Signal 4, ra70.
Xu, M. Z., Yao, T. J., Lee, N. P., Ng, I. O., Chan, Y. T., Zender, L., Lowe, S. W., Poon, R. T., and Luk, J. M. (2009). Yes-associated protein is an independent prognostic marker in hepatocellular carcinoma. Cancer 115, 4576-4585.
Xu, Y., Stamenkovic, I., and Yu, Q. (2010). CD44 attenuates activation of the Hippo-YAP signaling pathway and is a prime therapeutic target for glioblastoma. Cancer Res 70, 2455-2464.
Zender, L., Spector, M. S., Xue, W., Flemming, P., Cordon-Cardo, C., Silke, J., Fan, S. T., Luk, J. M., Wigler, M., Hannon, G. J., et al. (2006). Identification and validation of oncogenes in liver cancer using an integrative oncogenomic approach. Cell 125, 1253-1267.
Zhang, N., Bai, H., David, K. K., Dong, J., Zheng, Y., Cai, J., Giovannini, M., Liu, P., Anders, R. A., and Pan, D. (2010). The Merlin/NF2 tumor suppressor functions through the YAP oncoprotein to regulate tissue homeostasis in mammals. Dev Cell 19, 27-38.
Zhao, B., Li, L., Lei, Q., and Guan, K. L. (2010a). The Hippo-YAP pathway in organ size control and tumorigenesis: an updated version. Genes Dev 24, 862-874.
Zhao, B., Li, L., Tumaneng, K., Wang, C. Y., and Guan, K. L. (2010b). A coordinated phosphorylation by Lats and CK1 regulates YAP stability through SCF(beta-TRCP). Genes Dev 24, 72-85.
Zhao, B., Li, L., Wang, L., Wang, C. Y., Yu, J., and Guan, K. L. (2012). Cell detachment activates the Hippo-YAP pathway via cytoskeleton reorganization to induce anoikis. Genes Dev 26, 54-68.
Zhao, B., Wei, X., Li, W., Udan, R. S., Yang, Q., Kim, J., Xie, J., Ikenoue, T., Yu, J., Li, L., et al. (2007). Inactivation of YAP oncoprotein by the Hippo-YAP pathway is involved in cell contact inhibition and tissue growth control. Genes Dev 21, 2747-2761.
Zhao, B., Ye, X., Yu, J., Li, L., Li, W., Li, S., Lin, J. D., Wang, C. Y., Chinnaiyan, A. M., Lai, Z. C., et al. (2008). TEAD mediates YAP-dependent gene induction and growth control. Genes Dev 22, 1962-1971.
Zhou, D., Conrad, C., Xia, F., Park, J. S., Payer, B., Yin, Y., Lauwers, G. Y., Thasler, W., Lee, J. T., Avruch, J., et al. (2009). Mst1 and Mst2 maintain hepatocyte quiescence and suppress hepatocellular carcinoma development through inactivation of the Yapl oncogene. Cancer Cell 16, 425-438.

### Example 2

### Identification of YAP Inhibitors and their Use in Tumor Suppression

### Materials and Methods

Cell-Based Luciferase Screening.

For the luciferase reporter assay, BOCs cells were seeded in 15 cm dish. 5×UAS-luciferase reporter, YAP and TEAD4 plasmids were co-transfected as described previously. 16 hours after transfection, cells were splited to 384 well plates at the density of 10,000 cells/well using Multidrop (Thermo) cell dispenser. After allowing the cells to attach overnight, 10 μM of individual small molecule compounds were added using Biomek FXP Laboratory Automation Workstation (Beckman Coulter). Luciferase activity was assayed using the Dual-glo assay kit (Promega), and reporter activity was detected and quantified with plate reader at 560 nm.

Cell Culture, Chemical Treatment, RNA & RT-PCR, and Western Blot.

Human tumor cell lines were obtained from the American Type Culture Collection (ATCC) and maintained in the media and supplements according to recommended conditions. C108, MG132 and Cyclohexylamine (CHX) were formulated in Dimethyl sulfoxide (DMSO). RT-PCR was performed with SYBR green master mix in an ABI 3700 DNA detection system. Complementary DNA was synthesized with reverse transcriptase and random hexamoers (iScript, Bio-Rad laboratories) in a total volume of 20 μL from 1 μg of total RNA extracted with TRIZOL reagent. Primers for PCR amplification were described in our previous work (28). Expression levels of YAP were averaged from triplicates and normalized to the internal standard GAPDH using the standard delta-delta Ct method. Cell lysates and homogenized tumor tissues were analyzed by Western blot using antibodies from Cell Signaling (PARP, pYAP), Santa Cruz Biotechnologies (YAP, P53), and BD Biosciences (TAZ and alpha-Tubulin).

Wound Healing Assay.

Control and C108 treated cells were plated on fibronectin (10 mg/ml) coated 6 well plates in complete growth media. After overnight culture, wounds were made by manual scraping of the cell monolayer with a pipet tip. The dishes were washed, replenished with media in the presence or absence of C108, and photographed under phase contrast microscope. They were then placed in the tissue culture incubator, and the matched wound regions were photographed every 6 hrs. Migration rate is quantified by traveling distance of leading cellular edge normalized to initial gap distance.

Cell Migration Assay.

Cell migration assay was performed using BD Falcon™ Cell culture inserts for 24-well plates with 22.0 μm pores filter, and the filter was pre-coated with 10 mg/ml Fibronectin. Cancer cells were serum-starved for 24 h and then seeded into the upper chamber of the insert in serum-free media, and lower chamber was filled with media containing 20% FBS with or without 2 μM C108. After 24 h, cells were fixed using 4% paraformaldehyde and stained using 0.05% crystal violet. Cells in upper chamber were carefully removed, and cells migrated through the filter were assessed by photography. For quantification, crystal violet was extracted and the absorbance at 560 nm was measured.

Animal Studies.

Mouse procedures were performed according to the guidelines of approved animal protocol and based on the methods. Cells grown to 90% confluence were harvested by trypsinization, washed in phosphate-buffered saline (PBS), and resuspended in PBS supplemented with 50% matrigel (BD Biosciences). Cells in 200 μL per injection site were administered. Nude mice (Nu-foxn1nu, Charles Rivers Laboratories) were injected on both flanks of the dorsolateral sites subcutaneously. C108 was formulated in 10% DMSO and 40% propylene glycol then administered at a dose of 100 mg/kg daily via tail-vein injection. Control mice received vehicle alone. The average tumor diameter (two perpendicular axes of the tumor were measured) was recorded. The data are expressed in tumor volume estimated by ([width]²×length/2). Paired, two-tailed Student's t-test was performed to access the statistical significance.

### Results and Discussion

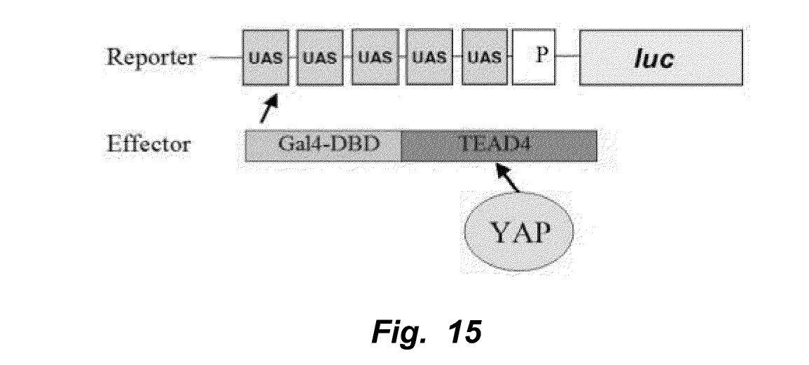

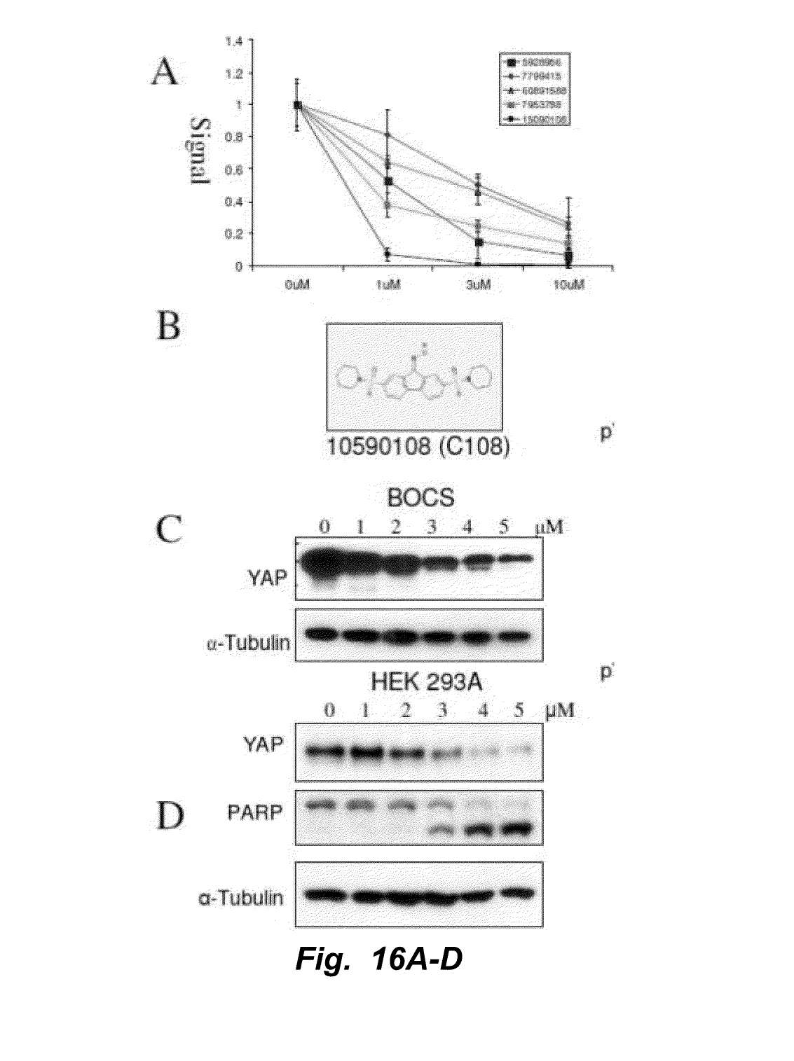

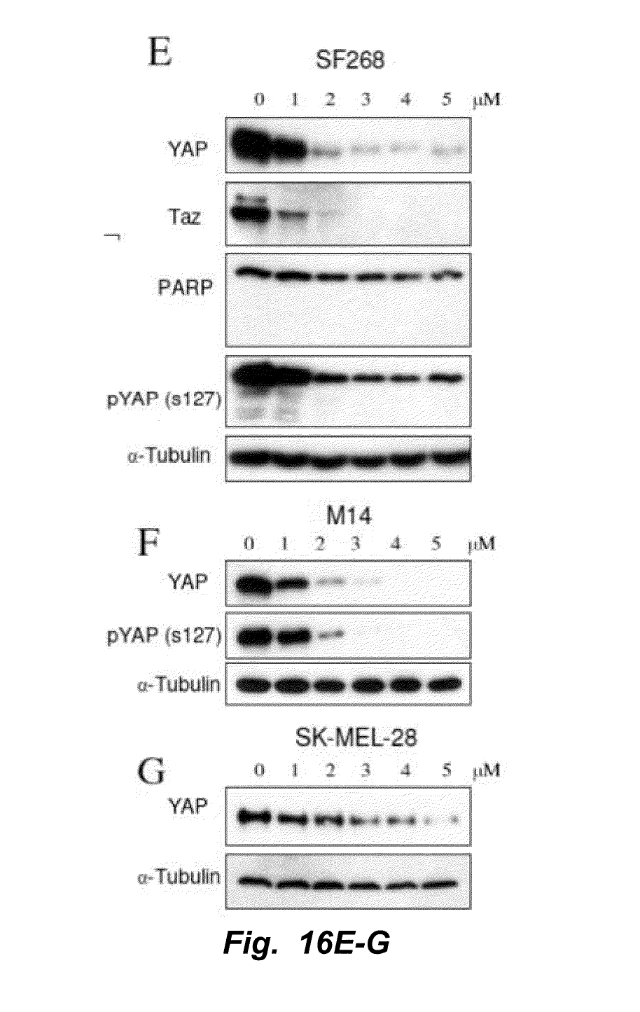

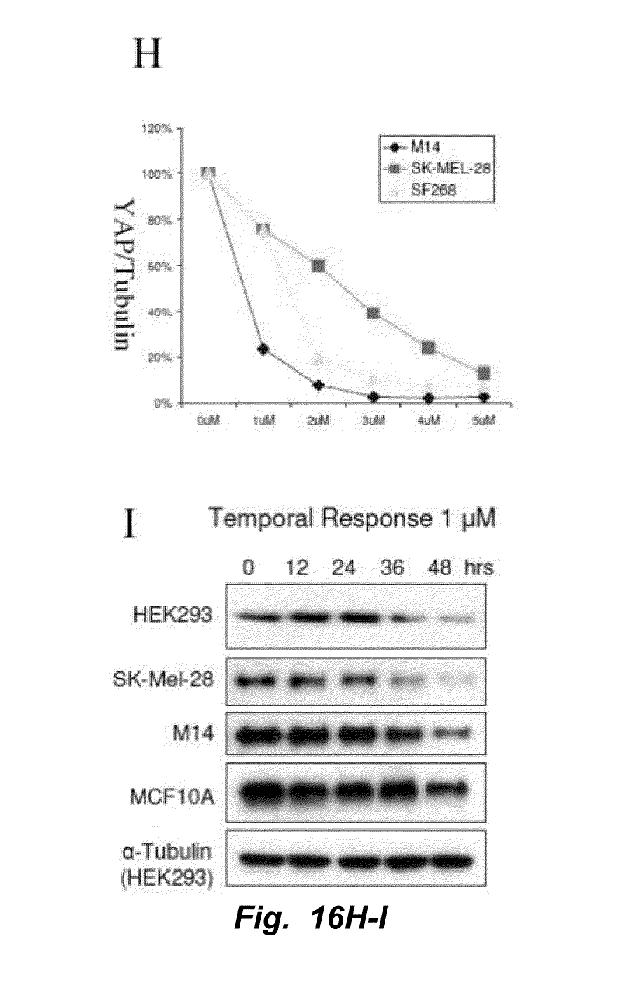

Inhibition of YAP and TAZ represents the major signaling output of the Hippo tumor suppressor pathway Inhibitors and activators of Hippo effecter YAP may emerge as new tools for cancer intervention. To search for small molecule compounds that may interfere the Hippo-YAP pathway, we established a sensitive cell-based YAP reporter assay, which consists of an UAS Luciferase reporter and a Gal4-fused TEAD transcription factor. This reporter activity is strongly stimulated by expression of YAP, which binds to and activates TEAD in transcription (FIG. 15). We improved this assay in BOCs cells, a derivation of Human Embryonic Kidney 293 cell line, to be tested in 384-well plates under the condition that both activators and inhibitors could be identified. Based on an HTS strategy that utilizes streamlined protocol including an automated robotic station, a diverse collection of more than 50,000 small molecules arrayed in 384-well plates as single compounds (at 10 mM in DMSO) was screened for their influence on the YAP reporter. After initial screen and further confirmation, we identified five compounds that consistently showed inhibitory effect on the YAP reporter in the BOCs cells in a dosage dependent manner (FIG. 16A).

Under physiological conditions, YAP is inactivated by Lats dependent phosphorylation, which promotes 14-3-3 binding and cytoplasmic localization. Decrease of the YAP reporter activity could be due to either activation of Lats or inhibition of YAP. In order to investigate whether these YAP-reporter inhibiting chemicals have any such roles, we examined the YAP phosphorylation status and found that they do not significantly increase relative YAP phosphorylation. Interestingly, 16 hours of treatment by one particular chemical (C108), but not the other inhibitors, resulted in a dramatic decrease of YAP protein levels in BOCS cells (FIG. 16C). C108 is an oxime derivative of 9-fluorenone bearing two piperidinylsulfonyl groups with MW of 498 Da (FIG. 16B). To determine if C108 decreases YAP protein levels in other cell types, multiple YAP expressing cancer cell lines, including SF268 glioma, M14 melanoma and SK-Mel-28 melanoma, were examined. Western blot results showed C108 similarly decreased YAP protein levels in these cancer cell lines (FIG. 16E-G) although some cell lines, such as M14, may be more sensitive than others, such as SK-MEL-28 (FIG. 16H, I). Collectively, these observations demonstrate the C108 inhibits YAP activity by decreasing YAP protein levels in a cell type independent manner.

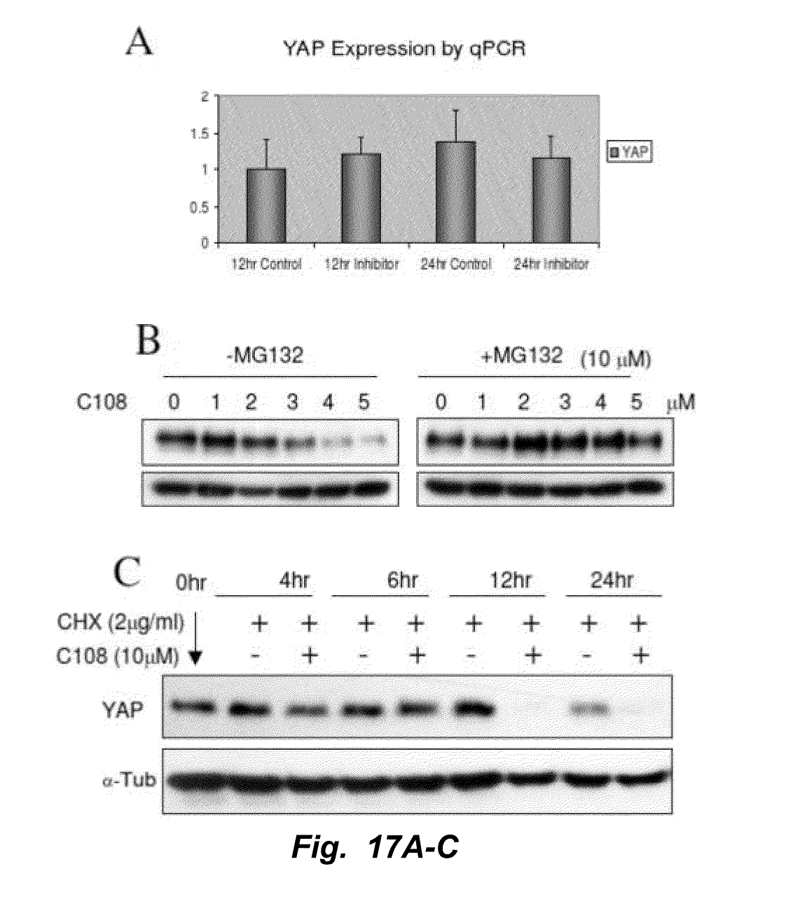

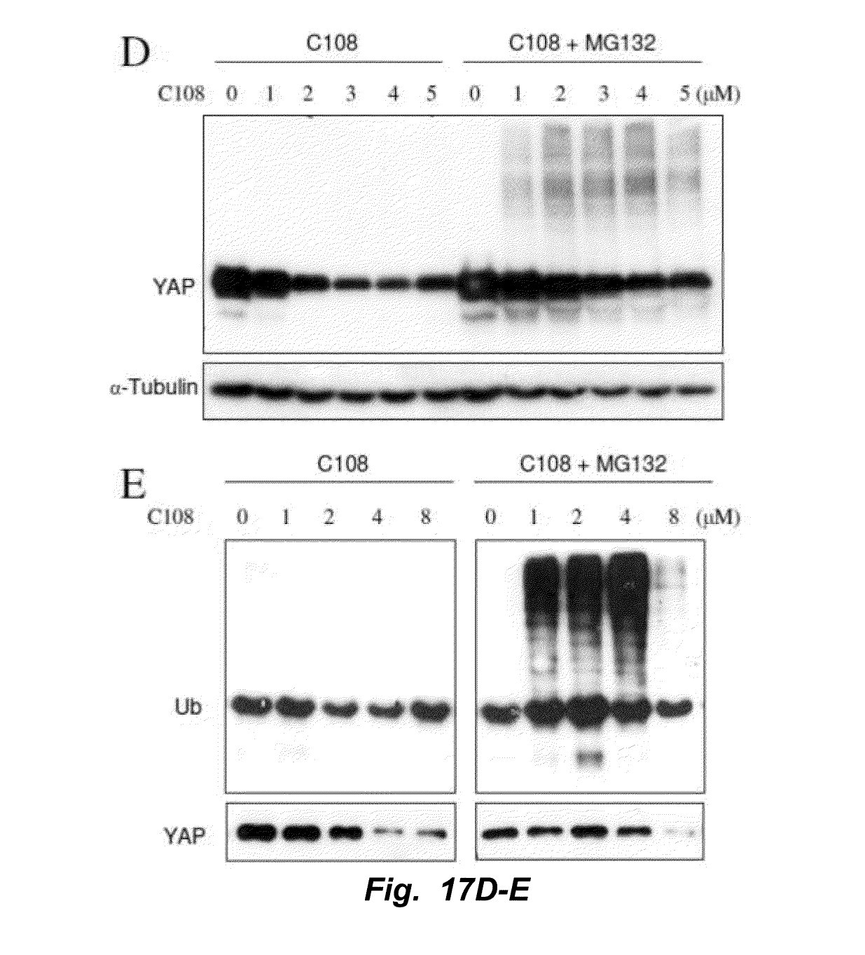

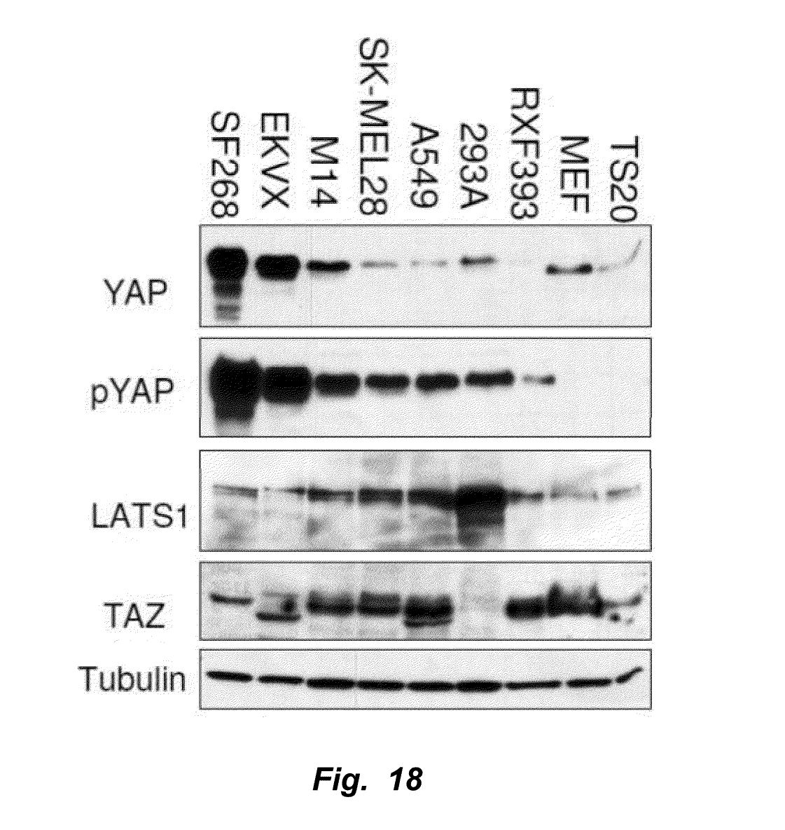

To investigate whether C108 decreases YAP expression at a transcription level, we determined YAP mRNA levels of BOCs 293 cells after 12 and 24 hr of drug exposure. While endogenous level of YAP protein was significantly reduced by C108 at 2-3 μM, no noticeable changes in YAP mRNA levels were observed at 3 μM (FIG. 17A), suggesting that C108 decreases YAP via posttranscriptional action. Our previous studies have also shown that YAP can be regulated by ubiquitination and proteasome mediated degradation (Zhao et al. 2010). When cells were co-treated with and without 10 μM proteasome inhibitor MG132 and various doses of C108, YAP protein levels were mostly maintained in the presence of MG132 (FIG. 17B), suggesting that proteasome mediated degradation is required for C108 to reduce YAP protein. Consistently, experiments utilizing cycloheximide, which inhibits the protein synthesis, showed that 10 μM of C108 rapidly diminished the YAP protein after 12 hrs of treatment (FIG. 17C). The ability of MG132 to block the effect of C108 on YAP protein levels suggests a role of proteasome dependent degradation. We next examined YAP ubiquitination using SF268, an aggressive human glioblastoma cell line containing high level of endogenous YAP protein (FIG. 18). Western blot for endogenous YAP showed slow-migrating immunoreactivity detected by the YAP antibody in the presence of C108 and MG132, indicative of YAP ubiquitination (FIG. 17D). To further support that the slow-migrating YAP was due to ubiquitination, endogenous YAP from SF268 was immuopercipitated followed by Western blotting with ubiquitin antibody. Experimental results showed that C108 treatment dramatically increased endogenous YAP ubiquitination (FIG. 17E). Taken together, the above data show that compound C108 inhibits YAP function and promotes YAP degradation through increasing YAP ubiquitination.

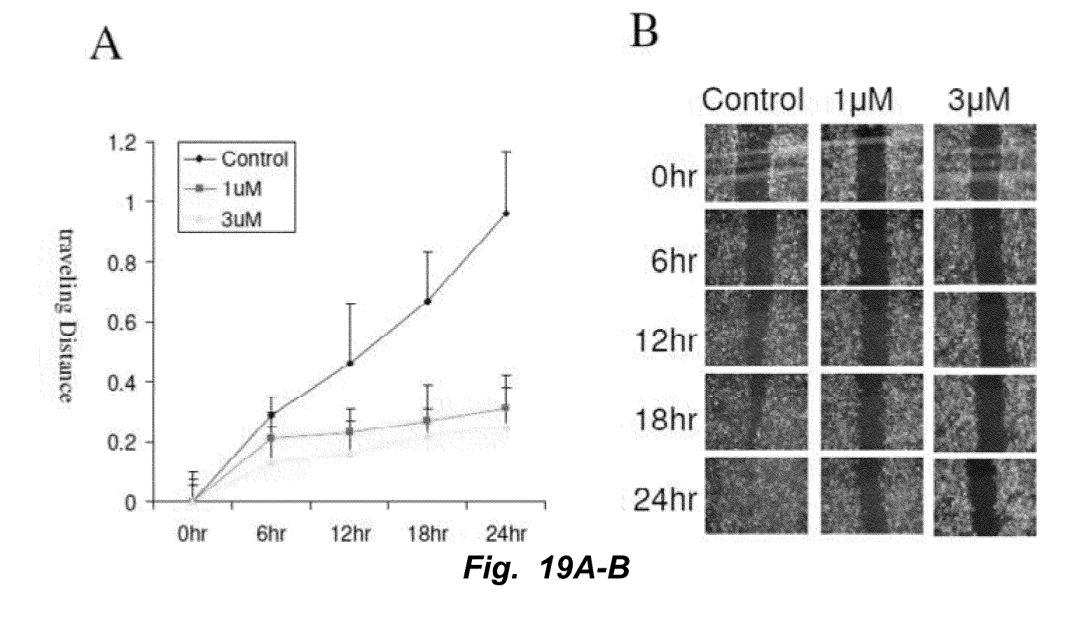

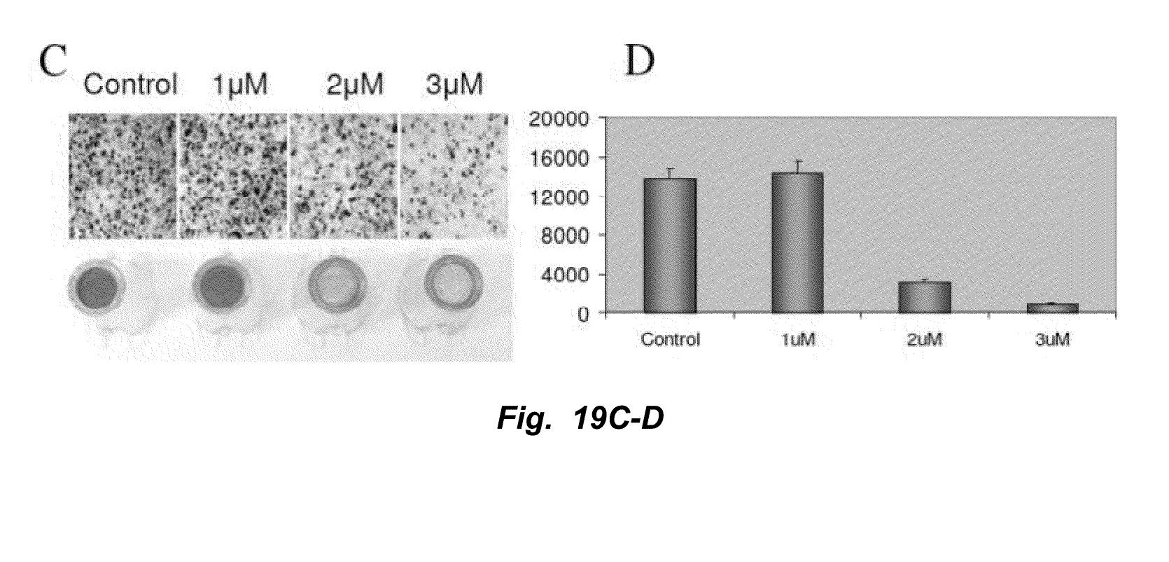

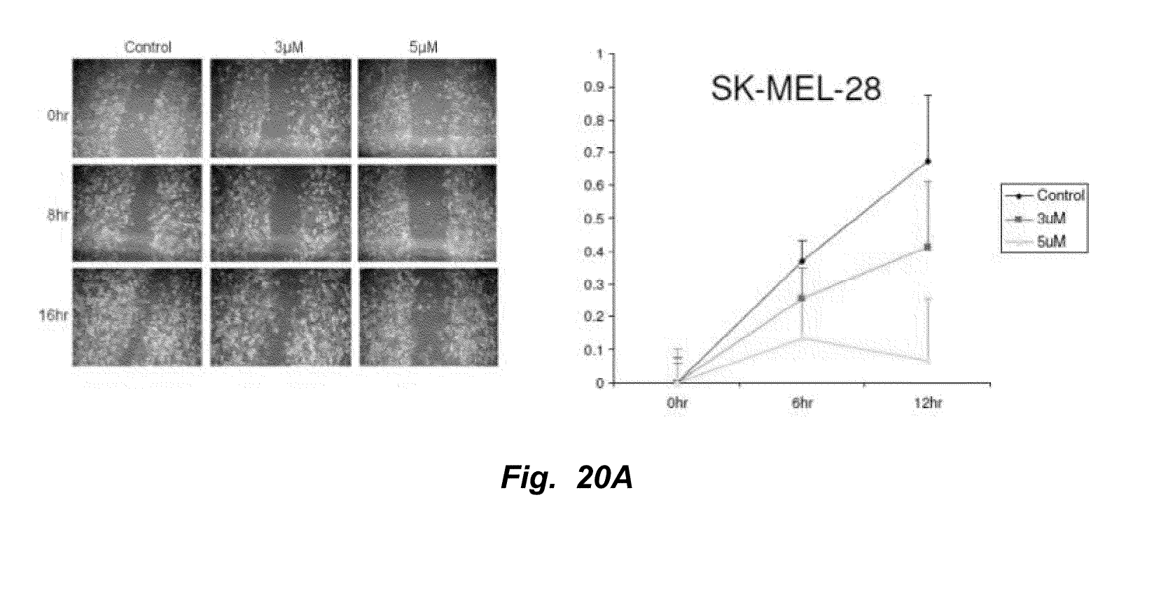

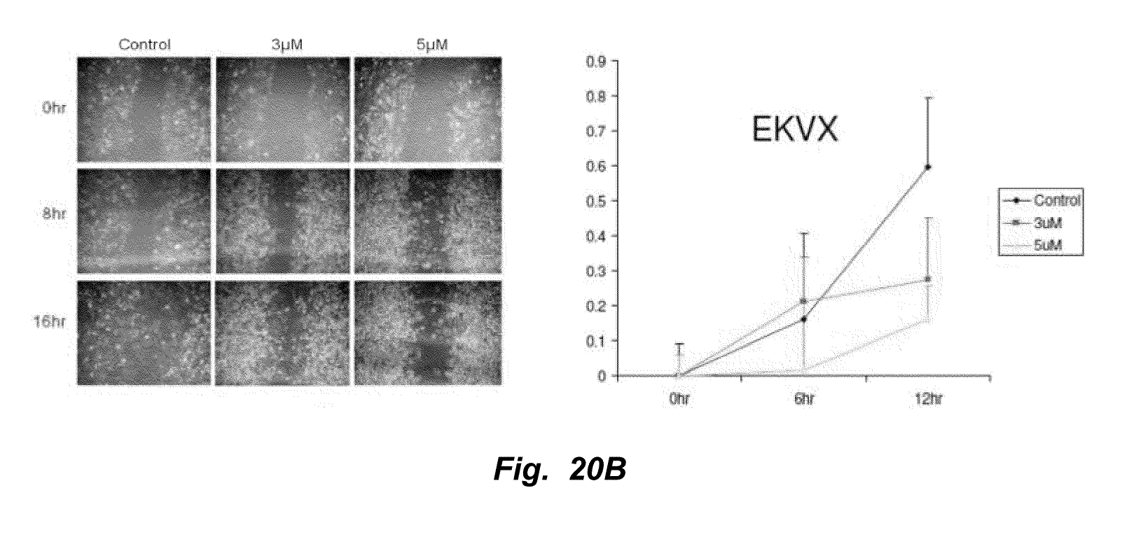

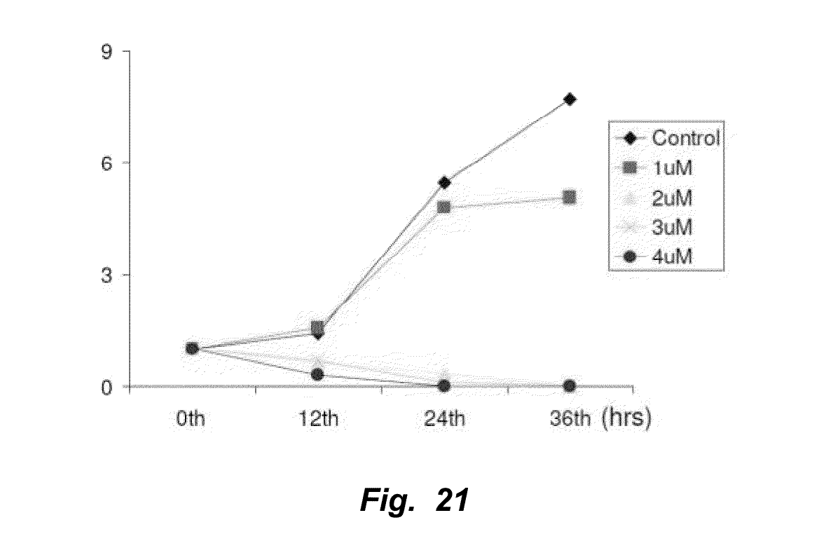

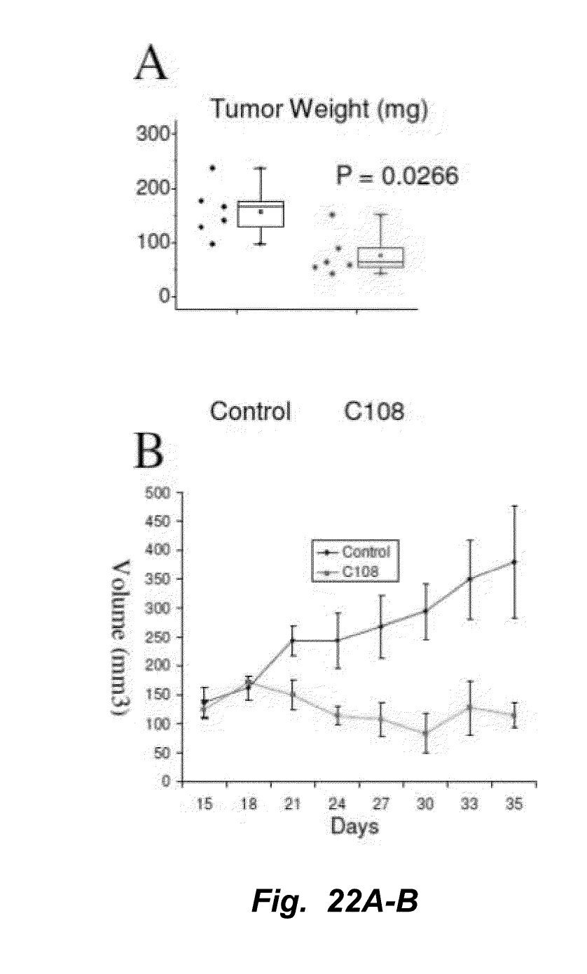

It has previously been shown that YAP can stimulate cell proliferation, migration, and tumorigenesis (Zhao et al. 2010). We examined the effect of C108 on the migration of human M14 cells, a tumorigenic melanoma line with abundant YAP expression (FIG. 18). As expected, C108 strongly inhibited cell migration in a wound-healing assay (FIGS. 19A and 19B). Furthermore, cell migration by a transwell assay was also tested. We found that C108 suppressed migration of M14 (FIG. 19C) as well as SK-Mel-28 melanoma and EKVX adenocarcinoma (FIG. 20). In addition, the inhibitory effect of C108 on M14 Melanoma cell proliferation was measured (FIG. 21). These data are consistent with the known function of YAP in cell proliferation and migration, suggesting a potential of targeting YAP to inhibit tumor cell growth and migration.

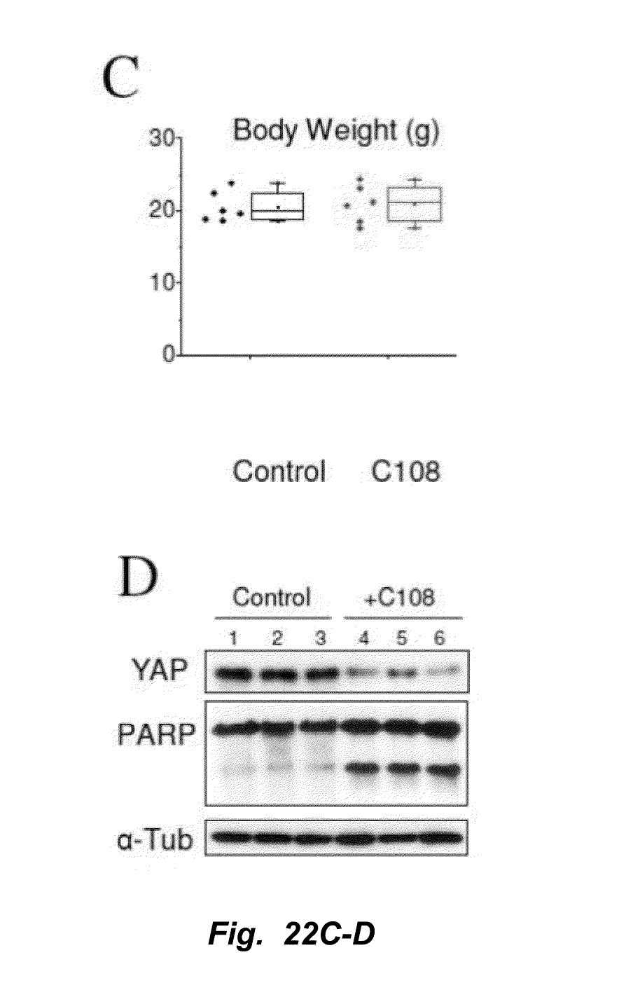

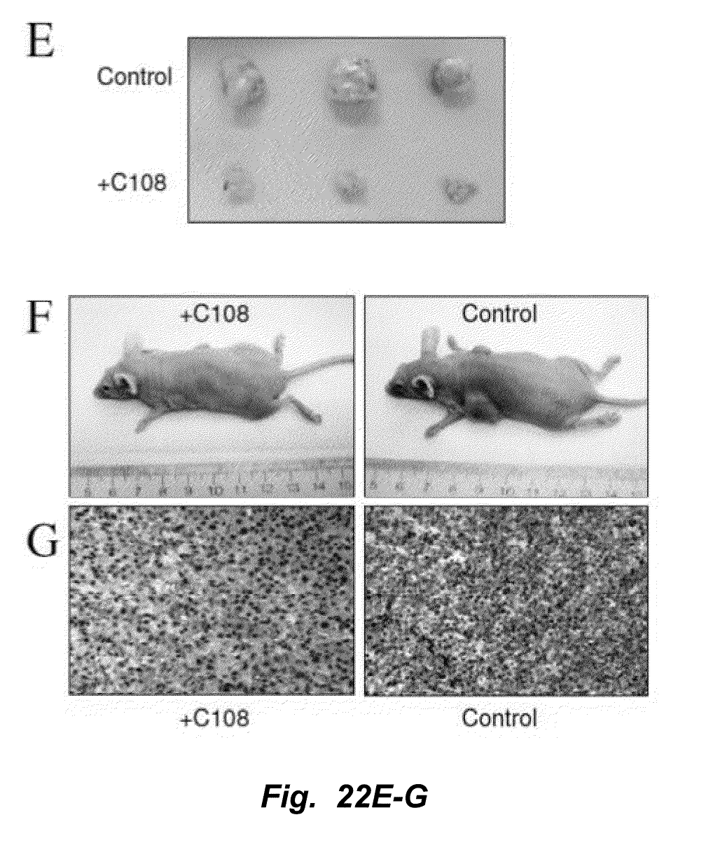

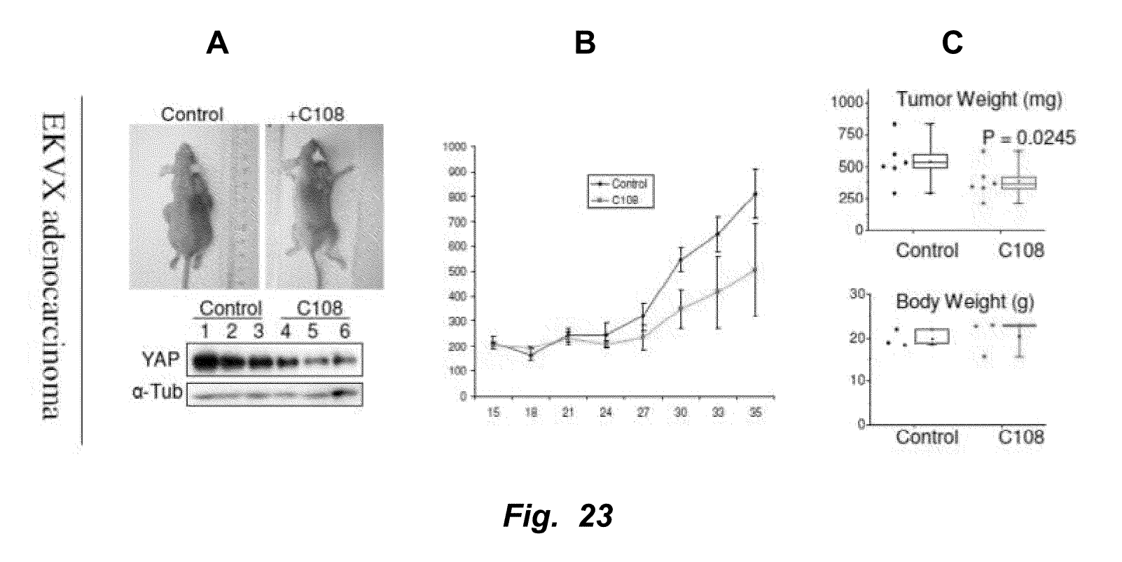

In order to investigate the physiological impact of C108 on implanted tumor cell growth, in vivo xenograft models using M14 melanoma and EKVX adenocarcinoma in immune compromised mice were examined. Cancer cells mixed with 50% matrigel were injected subcutaneously, palpable tumors with approximately 5 mm in diameter across all injection sites were observed within 3 weeks. At that point, control group (6 mice) was tail-vein injected with the solvent consist of 10% DMSO and 40% propylene glycol while the treatment group (6 mice) received daily injection of C108 for three additional weeks. During the treatment period, tumor growth was monitored and final tumor weights were measured. As shown in FIGS. 22A and 22B, C108 significantly inhibited growth of M14 and EKVX (FIG. 23) xenograft tumors. In contrast, total body weight was not affected by C108 (FIG. 22C). Further analysis of the tumor tissues by Western blot showed that samples from C108 treated mice displayed a lower level of YAP (FIG. 22D, top panel). These data are consistent with the in vitro observations and demonstrate that C108 could induce YAP degradation in vivo. Moreover, our Western blot results showed a dramatic increase of an apoptotic marker, cleaved PARP (FIG. 22D, middle panel), and in tumors from C108 treated animals, indicating that C108 treatment induced apoptosis of M14 tumor cells. Hematoxylin and Eosin stain (FIG. 22G) of the tumor sections showed that cells in melanoma xenografts subjected to C108 are sparser and have lower nuclear to cytoplasimc ratio, suggesting a less aggressive oncogenic nature.

Development of targeted anti-cancer therapeutics based on pathway specific inhibitors has proven to be an effective approach in the field of oncology research. Inhibitors of EGFR, Abl, PI3K, and BRAF have led to therapeutic treatment in multiple cancers (Collins and Workman 2006; Noro et al. 2006; Arora and Scholar 2005; Engelman 2009; Carnahan et al. 2010). In particular, a wide variety of market-approved regiments for malignancies such as acute promyelocytic leukemia harboring translocations in the RARα retinoic acid receptor, aoestrogens and androgens responsive breast and prostate cancers, EGFR responsive non-small cell lung cancer (NSCLC), vascular epidermal growth factor receptor (VEGFR) responsive renal cancer, as well as many others, have been developed from lead candidates via small molecule screens (Hoelders et al. 2012).

The Hippo-YAP pathway has been well established to regulate organ size, development, and tissue regeneration under physiological conditions. While deregulation of this pathway is known to contribute to tumorigenesis and emerges as a potential target for cancer therapeutics. To date inhibitors or activators that target this signaling pathway, particularly towards the YAP oncoprotein, have not been reported. In the current study, we identified a YAP inhibitor C108 via cell-based HTS that is able to modulate YAP protein levels by promoting ubiquitin-mediated degradation. C108 significantly inhibits proliferation and retards migration of multiple cancer cell lines in vitro. In addition, we presented mouse model data showing an anti-tumor potential for C108, which blocks xenograft melanoma and lung adenocarcinoma tumor growth and induces cancer cell apoptosis. Since YAP is known to suppress apoptosis and promote growth in general, inhibition of tumor growth as a consequence of YAP suppression is logical and in line with our observation. Moreover, our data provides pharmacological support for the function of YAP in promoting cell and tumor growth.

The identification of C108 may shed light into a new class of small molecules with novel functions. Our finding suggests a potential cancer therapeutic route by directly targeting YAP protein without incurring transcriptional change nor intervening upstream signaling of Hippo-YAP pathway. While C108 shows the capability of tumor suppression in vitro and in vivo in multiple cancer lines, the discovery of C108 along with other YAP-TEAD reporter attenuators revealed from our HTS work merely serve as a starting point for further screen for YAP inhibitors both for research and therapeutic potential. It should be cautioned that the antitumor efficacy of C108 in xenograft model might not directly translate to successful clinical outcome. Factors such as heterogeneity of tumor composition, presence of stroma and immune cells in human patients may present a very different microenvironment from the standard xenograft models done in immune compromised mice. C108 appears to be more potent for tumors with elevated YAP function. Additionally, modification in the compound structure for salt formation, optimization in solvent composition and delivery method may yet further improve the anti-oncogenic efficiency of our YAP inhibitor. Besides being a lead compound for cancer therapeutics, C108 may also serve as a valuable agent for research to investigate the biological function of YAP in cellular regulation. Since Hippo-YAP pathway plays important physiological roles beyond tumorigenesis, the implication of C108 as a tool to study those functions, such as growth and development, may also be significant. Our study not only is significant for revealing a novel candidate YAP suppressor, but also demonstrated the feasibility of therapeutically targeting the Hippo-YAP pathway.

### REFERENCES

Arora A & Scholar E M. 2005. Role of Tyrosine Kinase Inhibitors in Cancer Therapy. The Journal of Pharmacology and Experimental Therapeutics. 315, 971-979.
Camargo F D, Gokhale S, Johnnidis J B, Fu D, Bell G W, Jaenisch R, Brummelkamp T R. 2007. YAP1 increases organ size and expands undifferentiated progenitor cells. Curr Biol 17, 2054-2060.
Carnahan J, Beltran P J, Babij C, Le Q, Rose M J, Vonderfecht S, Kim J L, Smith A L, Nagapudi K, Broome M A, Fernando M, Kha H, Belmontes B, Radinsky R, Kendall R, Burgess T L. 2010. Selective and potent Raf inhibitors paradoxically stimulate normal cell proliferation and tumor growth. Mol Cancer Ther 9, 2399-410.
Collins I & Workman P. 2006. New approaches to molecular cancer therapeutics. Nature Chem Biol 12, 689-700.
Cordenonsi M, Zanconato F, Azzolinl L, Forcato M, Rosato A, Frasson C, Inuil M, Montagner M, Parenti A R, Poletti A, Daidone M G, SDupont S, Basso G, Bicciato S, Piccolol S. 2011. The Hippo Transducer TAZ Confers Cancer Stem Cell-Related Traits on Breast Cancer Cells. Cell 147(4) 11 759-772.
Dong J, Feldmann G, Huang J, Wu S, Zhang N, Comerford S A, Gayyed M F, Anders R A, Maitra A, Pan D. 2007. Elucidation of a universal size-control mechanism in Drosophila and mammals. Cell 130, 1120-1133.
Engelman J A. 2009. Targeting PI3K signalling in cancer: opportunities, challenges and limitations' Nature Reviews Cancer 9, 550-562.
Fernandez-L A, Northcott P A, Dalton J, Fraga C, Ellison D, Angers S, Taylor M D, Kenney A M. 2009. YAP1 is amplified and up-regulated in hedgehog-associated medulloblastomas and mediates Sonic hedgehog-driven neural precursor proliferation. Genes Dev 23, 2729-41.
Fossdal R, Jonasson F, Kristjansdottir G T, Kong A, Stefansson H, Gosh S, Gulcher J R. 2004. A novel TEAD1 mutation is the causative allele in Sveinsson's chorioretinal atrophy helicjoid peripapillary chorioretinal degeneration. Hum Mol Genet 1, 975-981.
Goulev Y, Fauny J D, Gonzalez-Marti B, Flagiello D, Silber J, Zider A. 2008. SCALLOPED interacts with YORKIE, the nuclear effector of the hippo tumor-suppressor pathway in Drosophila. Curr Biol. 18, 435-41.
Hoelder S, Clarke P A, Workman P. 2012. Discovery of small molecule cancer drugs: Successes, challenges and opportunities. Molecular Oncology. 6, 2 155-176.
Huang J, Wu S, Barrera J, Matthews K, Pan D. 2005. The Hippo-YAP signaling pathway coordinately regulates cell proliferation and apoptosis by inactivating Yorkie, the Drosophila Homolog of YAP. Cell 122, 421-434.
Kawano T, Inazawa J. 2011. YAP is a candidate oncogene for esophageal squamous cell carcinoma. Carcinogenesis 32(3), 389-398.
Kim J, Woo A J, Chu J, Snow J W, Fujiwara Y, Kim C G, Cantor A B, Orkin S H. 2010. A Myc network accounts for similarities between embryonic stem and cancer cell transcription programs. Cell 143(2), 313-24.
Lian I, Kim J, Okazawa H, Zhao J, Zhao B, Yu J, Chinnaiyan A, Israel M A, Goldstein L S B, Abujarour R, Ding S, Guan K L. 2010. The role of YAP transcriptoin coactivator in regulating stem cell self-renewal and differentiation. Genes Dev 24, 1106-1118.
Lin J K, Zhang X, Harvey K F. 2011. The sterile 20-like kinase Tao-1 controls tissue growth by regulating the Salvador-Warts-Hippo-YAP pathway. Dev Cell 21, 896-906.
Liu A M, Xu M Z, Chen J, Poon R T, Luk J M. 2010. Targeting YAP and Hippo-YAP signaling pathway in liver cancer. Expert Opin Ther Targets. 8, 855-68.
Martis E A, Radhakrishnan R, Badve R R. 2010. High-Throughput Screening: The Hits and Leads of Drug Discovery—An Overview. Journal of Applied Pharmaceutical Science 01 (01); 2011: 02-10.
Neto-Silva R M, de Beco S, Johnston L A. 2010. Evidence for a growth-stabilizing regulatory feedback mechanism between Myc and Yorkie, the Drosophila homolog of Yap. Dev Cell 4, 507-520.
Noro R, Gemma A, Kosaihira S, Kokubo Y, Chen M, Seike M, Kataoka K, Matsuda K, Okano T, Minegishi Y, Yoshimura A, Kudoh S. 2006. Gefitinib (IRESSA) sensitive lung cancer cell lines show phosphorylation of Akt without ligand stimulation. BMC Cancer 6, 277.
Overholtzer M, Zhang J, Smolen G A, Muir B, Li W, Sgroi D C, Deng C X, Brugge J S, Haber D A. 2006. Transforming properties of YAP, a candidate oncogene on the chromosome 11q22 amplicon. Proc. Natl. Acad. Sci. 103, 12405-12410.
Ren F, Zhang L, Jiang. 2009. J. Hippo signaling regulates Yorkie nuclear localization and activity through 14-3-3 dependent and independent mechanisms. Dev Biol 337, 303-312.
Rich J, Bao S. 2011. Chemotherapy and Cancer Stem Cells. Cell Stem Cell 1(4) 11 353-355.
Saucedo L J, Edgar B A. 2007. Filling out the Hippo-YAP pathway. Nat. Rev. Mol. Cell Biol. 8, 613-21.
Tamm C, Bower N, Anneren C. 2011. Regulation of mouse embryonic stem cell self-renewal by a Yes-YAP-TEAD2 signaling pathway downstream of LIF. J. of Cell Sci. 124, 1136-1144.
Wei X, Shimizu T, Lai Z C. 2007. Mob as tumor suppressor is activated by Hippo kinase for growth inhibition in. Drosophila. EMBO J 26, 1772-81.
Xu M, Yao T J, Lee N, Ng I, MD Chan Y T, Zender L W, Lowe S W, Poon R, Luk J M. 2009. Yes-Associated Protein Is an Independent Prognostic Marker in Hepatocellular Carcinoma. Cancer 115, 19 4576-4585.
Zender L, Spector M S, Xue W, Flemming P, Cordon-Cardo C, Silke J, Fan S T, Luk J M, Wigler M, Hannon G J, et al. 2006. Identification and validation of oncogenes in liver cancer using an integrative oncogenomic approach. Cell 125, 1253-1267.
Zhang X, George J, Deb S, Degoutin J L, Takano E A, Fox, AOCS Study group, Bowtell D L, Harvey K F. 2011. The Hippo-YAP pathway transcriptional co-activator, YAP, is an ovarian cancer oncogene. Oncogene 30, 2810-2822.
Zhang L, Ren F, Zhangm Q, Chen Y, Wang B, Jiang J. 2008. The TEAD/TEF family of transcription factor Scalloped mediates Hippo signaling in organ size control. Dev Cell 14, 377-87.
Zhao B, Li L, Lei Q, Guan K L. 2010. The Hippo-YAP pathway in organ size control and tumorigenesis: an updated version. Genes Dev 24, 862-874.
Zhao B, Li L, Tumaneng K, Wang C U, Guan K L. 2010. A coordinated phosphorylation by Lats and CK1 regulates YAP stability through SCFβ-TRCP. Genes Dev 24, 72-85.
Zhao B, Li L, Wang L, Wang C U, Yu J, Guan K L. 2012. Cell detachment activates the Hippo-YAP pathway via cytoskeleton reorganization to induce anoikis. Genes & Dev. 26: 54-68.
Zhao B, Ye X, Yu J, Li L, Li W, Li S, Yu J, Lin J D, Wang C Y, Chinnaiyan A M, Lai Z C, Guan K L. 2008. TEAD mediates YAP-dependent gene induction and growth control. TEAD mediates YAP-dependent gene induction and growth control. Gene Dev 22, 1962-1971.

### Example 3

### Protein Kinase A Activates the Hippo-YAP Pathway to Modulate Cell Proliferation and Differentiation

Recently, we have demonstrated that extracellular diffusible signals modulate the Hippo-YAP pathway through G-protein coupled receptor (GPCR) signaling (Mo et al. 2012; Yu et al. 2012a; Yu et al. 2012b). GPCR is the largest family of cell surface receptors encoded in the human genome and has been implicated in almost every aspect of physiological regulation. We observed that hormonal factors like LPA, S1P, and Thrombin can activate Gα_{12/13} to stimulate YAP/TAZ, which mediate the effect of these signals on gene expression, cell proliferation and migration (Mo et al. 2012; Yu et al. 2012b). Similar observations were also reported by Wu and colleagues (Miller et al. 2012). In contrast, ligands of Gαₛ-coupled receptors, such as epinephrine and glucagon, stimulate Lats1/2 and result in inhibition of YAP/TAZ (Yu et al. 2012b). These findings suggest that the activity of YAP/TAZ can be positively or negatively modulated by a wide range of extracellular signals via GPCRs in a manner dependent on which intracellular signaling pathway is stimulated.

Activation of Gαₛ-coupled receptors usually results in accumulation of cyclic adenosine monophosphate (cAMP), an important second messenger with diverse physiological functions including cell proliferation and differentiation (Cho-Chung 1990). Despite extensive studies, the precise molecular mechanisms of how cAMP regulates cell proliferation and differentiation is not fully understood (Stork and Schmitt 2002). In this example, we demonstrate that cAMP acts through protein kinase A (PKA, cAMP-dependent protein kinase) and Rho GTPases to stimulate Lats kinase activity and inhibit YAP/TAZ. Inhibition of YAP/TAZ is critical for cAMP and PKA to promote adipogenesis and suppress growth, establishing the Hippo-YAP as a signaling branch downstream of cAMP and PKA.

### Materials and Methods

Cell Culture.

MDA-MB-231 cells were cultured in DMEM/F12 medium (Invitrogen). HEK239A, HEK293T, U2OS and MEF were cultured in DMEM medium (Hyclone). Primary hepatocytes were isolated from 12 weeks old male mice using a standard protocol and incubated in DMEM medium. All of the above cells were supplemented with 10% FBS (Omega Scientific) and 50 μg/mL penicillin/streptomycin (P/S). MCF10A cells were cultured in DMEM/F12 supplemented with 5% horse serum (Invitrogen), 20 ng/mL EGF, 0.5 μg/mL hydrocortisone, 10 μg/mL insulin, 100 ng/mL cholera toxin, and 50 μg/mL P/S. For serum starvation, cells were incubated in DMEM or DMEM/F12 without supplements. All cell lines were maintained at 37° C. with 5% CO₂.

Chemicals.

Epinephrine, glucagon, dexamethasone, troglitazone and rolipram were purchased from Sigma Aldrich. IBMX, forskolin, KT5720, ibudilast, theophylline were purchased from Tocris.

Transfection.

Cells were transfected with plasmid DNA using PolyJet™ DNA In Vitro Transfection Reagent (Signagen Laboratories) according to manufacturer's instruction. Dr. Mark Ginsberg (UCSD) generously provided GFP-GDI plasmid. pCMV-Flag-YAP, pCDNA3-MST2 K/R and pCDNA3-Lats2 K/R plasmids have been described elsewhere (Zhao et al. 2010; Zhao et al. 2012). RhoA, PKA catalytic subunit and Rap1b were in a pCDNA3 vector. PKA regulatory subunit mutants (Iα and IIα) were in a pEGFP-C1 vector.

RNAi.

Smartpool siRNAs were purchased from Dharmacon, siRNAs were delivered into cells using RNAiMAX (Invitrogen) according to manufacturer's instructions. Lentiviral shRNAs in pLKO.1 vector were purchased from Sigma Aldrich, and virus was made in HEK293T cells using pMD2.g and PsPAX2 as packaging plasmids. Virus was filtered and used to infect targeting cells. The TRC IDs for shRNAs used for PKA catalytic subunit (PRKACA) are TRCN0000001372 and TRCN0000001373.

Immunoblotting.

Immunoblotting was performed using standard protocol. Antibodies for pYAP (S127), YAP, TAZ (V386), pCREB, CREB, pMLC2 (S19), pMLC2 (T18/S19), MST1, MST2, and Lats1 were from Cell Signaling Technology. Lats2 antibody was from Bethyl laboratories. HA-HRP, GFP, and MLC2 antibodies were from Santa Cruz biotechnology. Tubulin, HSP90 and Flag-HRP were purchased from Sigma Aldrich. PKA antibody was obtained from BD biosciences. GAPDH antibody was a gift from Dr. Yan Luo. Yki antibody was a gift from Dr. Kenneth Irvine. The phos-tag reagents were purchased from Wako Chemicals, and gels containing phos-tag and MnC12 were prepared according to manufacturer's instructions. YAP proteins can be separated into multiple bands on phos-tag gels, with the phosphorylated form of YAP proteins migrating at a slower speed.

Immunoprecipitation and Lats Kinase Assay.

Cells were lysed using mild lysis buffer (50 mM HEPES at pH 7.5, 150 mM NaCl, 1 mM EDTA, 1% NP-40, 10 mM pyrophosphate, 10 mM glycerophosphate, 50 mM NaF, 1.5 mM Na3VO4, protease inhibitor cocktail [Roche], 1 mM PMSF). Cell lysates were cleared and used for immunoprecipitation. Lats1 antibody (Cell Signaling Technology) was mixed with cell lysates for 1 hr at 4° C., and then protein A agarose beads were added in for 1 hr. After four washes with lysis buffer, beads were washed once with wash buffer (40 mM HEPES, 200 mM NaCl) and once with kinase assay buffer (30 mM HEPES, 50 mM potassium acetate, 5 mM MgCl2). The immunoprecipitated Lats1 was then subjected to a kinase assay in the presence of 500 μM cold ATP, 10 μCi [γ-32P] ATP, and 1 μg of GST-YAP expressed and purified from Escherichia coli as substrate. The reaction mixtures were incubated for 30 min at 30° C., terminated with SDS sample buffer, and subjected to SDS-PAGE and immunoblotting.

RNA extraction, reverse transcription and real-time PCR. Following forskolin treatments or adipogenesis, cells were washed with cold phosphate-buffered saline and subjected to RNA extraction using an RNeasy Plus mini kit (Qiagen). RNA samples (1 μg) were reverse-transcribed to complementary DNA (cDNA) using iScript reverse transcriptase (Bio-Rad). After dilution, cDNA levels were quantified by real-time PCR using KAPA SYBR FAST qPCR master mix (Kapa Biosystems) and the 7300 real-time PCR system (Applied Biosystems). Primer pairs (h and m indicate human and mouse, respectively) used in this study are:

β-actin (h):
GCCGACAGGATGCAGAAGGAGATCA/
AAGCATTTGCGGTGGACGATGGA
CTGF (h):
CCAATGACAACGCCTCCTG/TGGTGCAGCCAGAAAGCTC
HPRT (m):
GCAGTACAGCCCCAAAATGG/ACAAAGTCCGGCCTGTATCCAA
C/EBPα (m):
GCAAAGCCAAGAAGTCGGTGGA/CCTTCTGTTGCGTCTCCACGTT
PPARγ (m):
CTGTCGGTTTCAGAAGTGCCT/CCCAAACCTGATGGCATTGTGAGACA
Adiposin (m):
TCCGCCCCTGAACCCTACAA/TAATGGTGACTACCCCGTCA
FABP4 (m):
CGATGAAATCACCGCAGACGA/AGTCACGCCTTTCATAACACA
Adoponectin (m):
AGATGGCACTCCTGGAGAGAAG/ACATAAGCGGCTTCTCCAGGCT

Adipogenesis. Murine 3T3-L1 cells were maintained in DMEM medium containing 10% calf serum (Hyclone). To initiate adipocyte differentiation, confluent 3T3-L1 cells switched into FBS containing DMEM medium; in addition, insulin, dexamethasone, troglitazone (Tro) were added; in selected samples, IBMX (250 μM) or forskolin (100 μM) were used to increase cAMP and PKA activity. For experiments using KT5720, 3T3-L1 cells were pre-treated with KT5720 (5 μM) overnight in advance and fresh KT5720 was added when adipogenesis was initiated. Two days later, medium was changed into DMEM containing 10% FBS and insulin. And after another two days, cells were incubated in DMEM with FBS. Cells were typically harvested on day 6 depending on the formation and maturation of lipid droplets. Cells were then subjected to RNA extraction or oil red (Sigma Aldrich) staining according to manufacturer's instructions.

Luciferase assay. S2R+ cells were cultured in 24-well plates at standard condition. PKA-C1 was down regulated by dsRNAs. All the samples were co-transfected with 10 ng of the copia-Renilla luciferase reporter as a normalization control and 200 ng of 3xSd_luc (gift from Dr. Jin Jiang) firefly luciferase reporter using Cellfectin II (Invitrogen). 50 ng of pUAST-Yki, pUAST-HA-sd and pAc-Gal4 each were used in each well to promote the firefly Luciferase expression. Luciferase activity was measured after 48 hr incubation using Dual-Glo™ luciferase assay kit (Promega) according to the manufacturer's protocol.

Drosophila stocks, quantitative RT-PCR and immunocytochemistry. All fruit flies were maintained under standard conditions. Fly stocks UAS-PKA-C1 (ID#35554) and UAS-PKA-C1-RNAi (ID#31277) were obtained from Bloomington Drosophila Stock Center. Total mRNA from Drosophila late third-instar larval wing discs was isolated using Qiagen RNAeasy Kit (Qiagen) and mRNA was reverse-transcribed using Quanta qScriptcDNAsuperMix (Qiagen). Real-time PCR was performed using PerfeCTA SYBR Green FastMix (Qiagen) and data was collected via Applied Biosystem StepOnePlus Real-Time PCR system (Life Technologies). The relative amount of specific mRNAs under each condition was calculated after normalization to the Histone 3 transcripts. Standard procedure for immunocytochemistry was followed in this study. Wing discs from late third-instar larvae were dissected in cold PBS, fixed in 4% PFA for 30 min at room temperature. Tissues were incubated in primary antibody at 4° C. overnight, followed by 2 hr secondary antibody incubation at room temperature. Primary antibodies anti-Diap1 mouse (1:200) (gift from Dr. Bruce Hay) and anti-Caspase-3 Rabbit (1:200) (Cell Signaling Technology) were used in this study. Images were collected with an Olympus Fluoview 1000 Confocal Laser Scanning Microscope.

### Results

cAMP Signaling Stimulates YAP Phosphorylation.

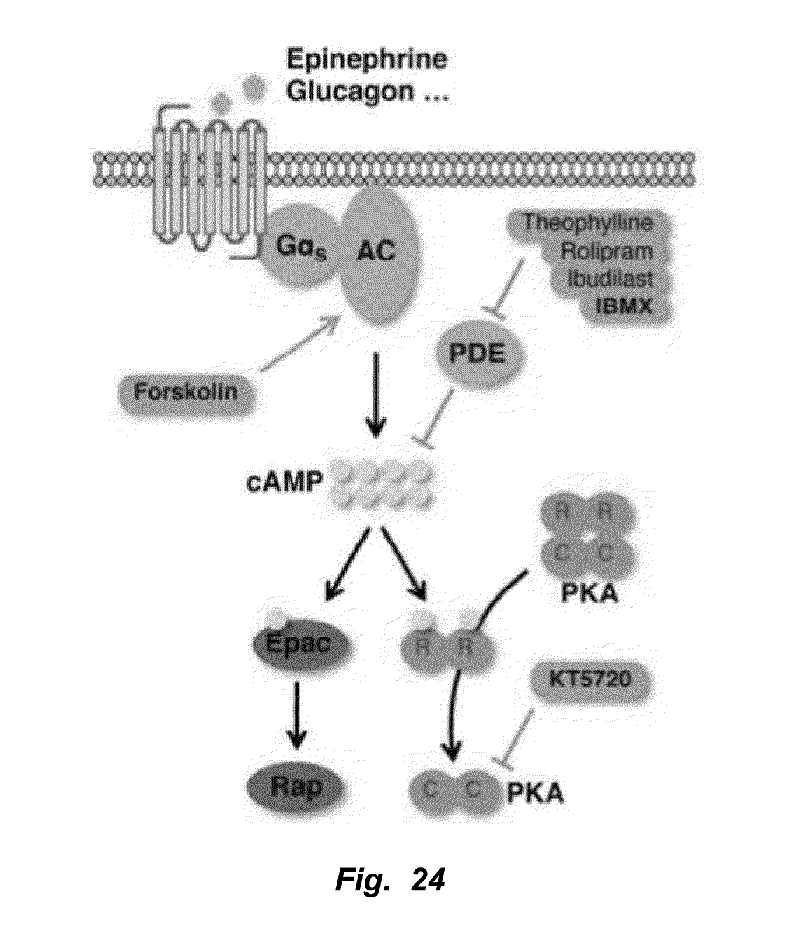

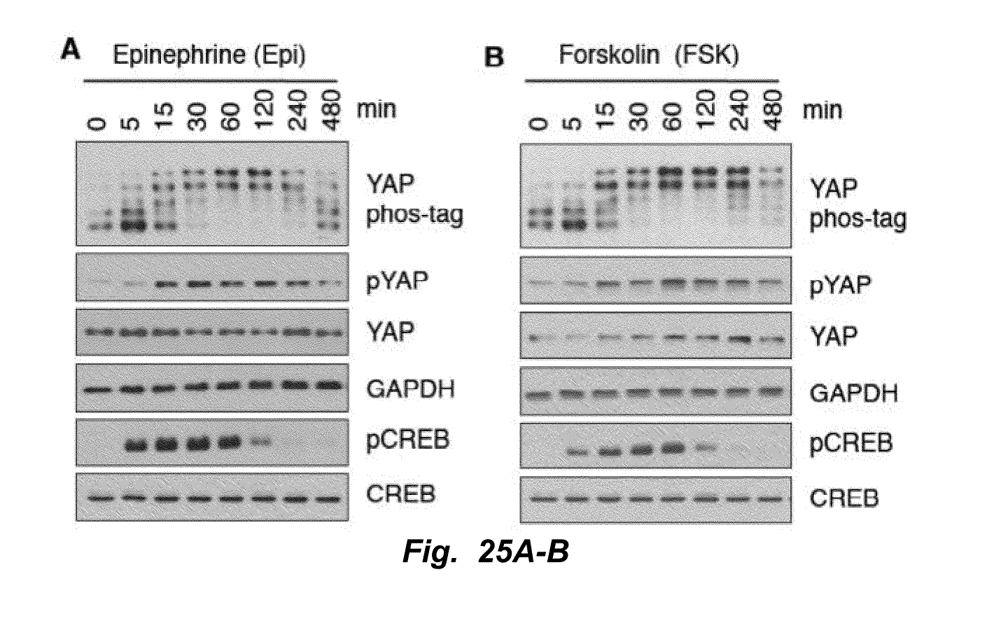

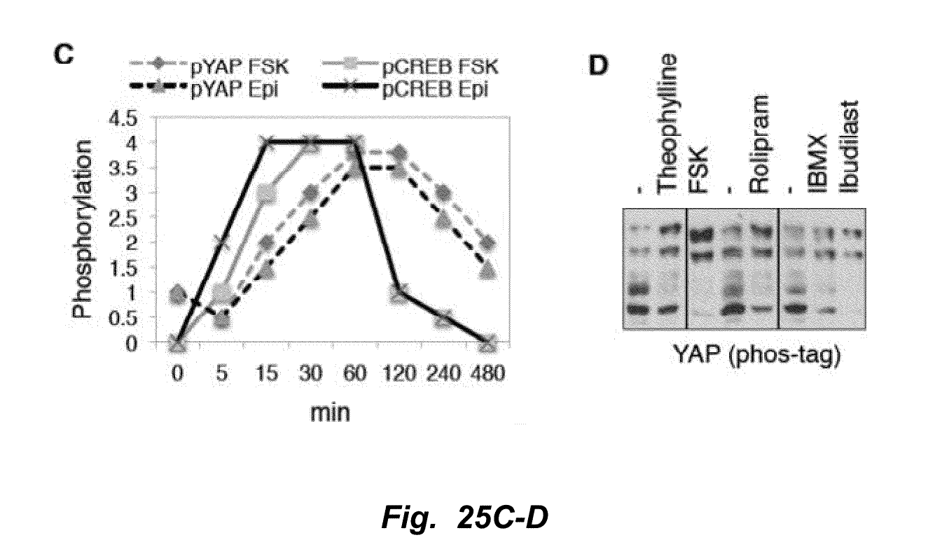

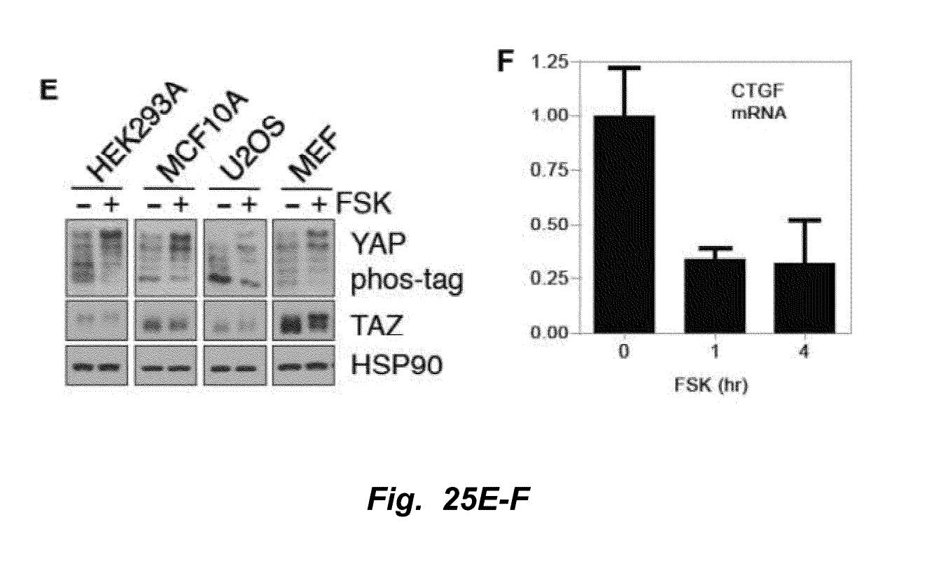

Activation of Gαₛ-coupled receptors can stimulate adenylyl cyclase (AC) and result in an increase of cAMP production (Sassone-Corsi 2012). We treated MDA-MB-231 breast cancer cells with epinephrine, a ligand for β2 adrenergic receptor that increases cAMP (FIG. 24). As anticipated, we observed a transient induction of phosphorylation of the cAMP response element-binding protein (CREB), a direct target of PKA and an indicator of cAMP accumulation and PKA activation (FIG. 25A). Interestingly, YAP phosphorylation was also transiently increased in response to epinephrine, as assessed by a phosphospecific antibody against Ser127, which is a direct Lats phosphorylation site responsible for cytoplasmic localization, or a phos-tag gel, which resolves YAP protein based on phosphorylation status (FIG. 25A). When cellular cAMP was induced by forskolin, a pharmacological activator of adenylyl cyclase (AC) (FIG. 24), phosphorylation of both YAP and CREB was similarly induced as seen with epinephrine treatment (FIG. 25B). The phosphorylation of YAP in response to cAMP was maximum at 1 hr and started to decline at 4 hr. Notably, the response of CREB to cAMP singling was swifter (FIG. 25C), suggesting that YAP and CREB might be regulated by different molecular mechanisms downstream of cAMP (see below).

Intracellular cAMP levels are controlled by both biosynthesis and degradation. In mammalian cells, multiple phosphodiesterases (PDEs) are able to breakdown cAMP (FIG. 24). Many pharmaceutical drugs are direct PDE inhibitors that can be used to increase cellular cAMP levels (FIG. 24) (Sassone-Corsi 2012). Several nonselective phosphodiesterase inhibitors (theophylline, IBMX and ibudilast) and PDE4 selective inhibitors (rolipram) all induced YAP phosphorylation (FIG. 25D), further supporting the role of cAMP in stimulating YAP phosphorylation. In addition, these data also suggest that PDE inhibitors might be useful tools for restricting YAP activity.

We have tested the effect of forskolin on YAP phosphorylation in multiple cell lines including U2OS, MCF10A, HEK293A and mouse embryonic fibroblast (MEF). In all cases, YAP phosphorylation was increased by forskolin treatment (FIG. 25E). TAZ is a YAP homolog similarly regulated by the Hippo-YAP pathway (Lei et al. 2008). As expected, TAZ phosphorylation was increased as indicated by the decreased electrophoretic mobility (FIG. 25E). Moreover, TAZ protein levels were modestly reduced in forskolin treated cells because TAZ is destabilized by phosphorylation (Liu et al. 2010). These results indicate that the crosstalk between cAMP signaling and the Hippo-YAP pathway is a conserved phenomenon in different cell types, and both Hippo-YAP pathway effectors, YAP and TAZ, are inactivated by cAMP.

YAP/TAZ are transcriptional co-activators. To determine the functional significance of intracellular cAMP on YAP activity, we determined expression of YAP/TAZ target genes. Indeed the expression of CTGF, which is a direct YAP/TAZ target gene, was inhibited by forskolin in MCF10A cells (FIG. 25F), further supporting the idea that cAMP inhibits YAP and TAZ activity.

cAMP Signals Through PKA to Stimulate YAP Phosphorylation.

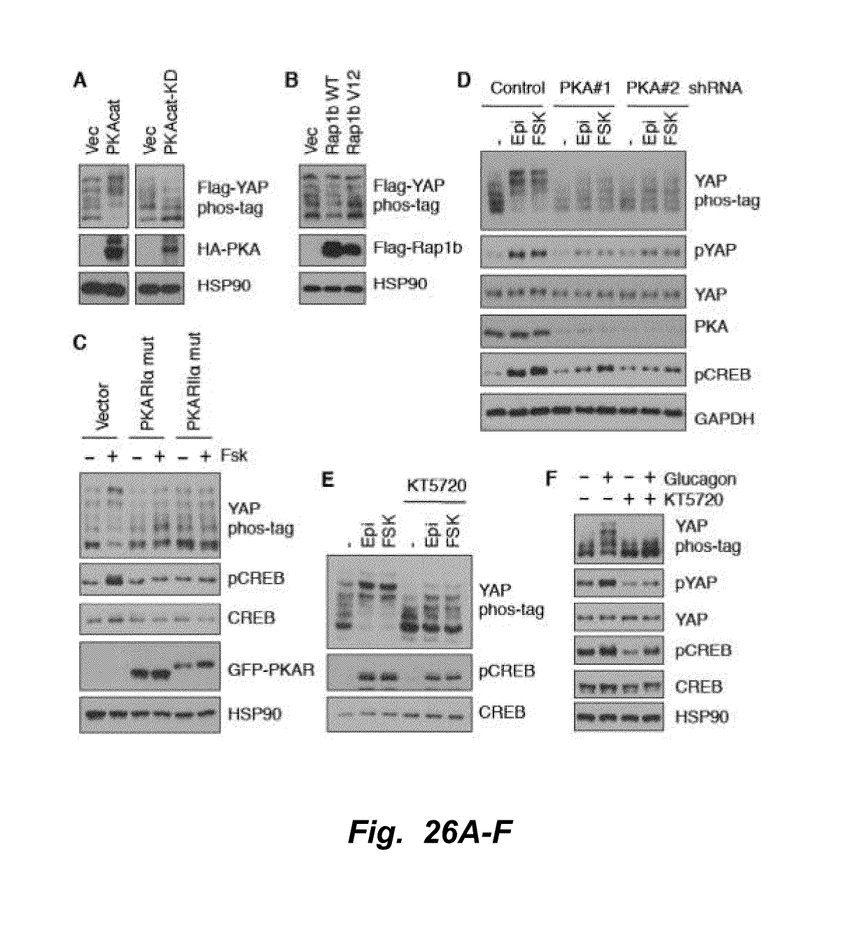

Exchange protein activated by cAMP (Epac) and PKA are two downstream effectors mediating most physiological functions of cAMP (FIG. 24). Epac proteins and the regulatory (R) subunits of PKA contain cAMP-binding domains that function as cAMP sensors (Gloerich and Bos 2010; Taylor et al. 2012). We investigated whether PKA or Epac signaling mediated the effect of cAMP on YAP phosphorylation. Overexpression of the catalytic (C) subunit alpha (PRKACA) induced YAP phosphorylation, whereas overexpression of the PKA kinase-dead mutant decreased YAP phosphorylation (FIG. 26A). In contrast, overexpression of wild type or constitutively active Rap1b, an effector of Epac (Gloerich and Bos 2010), did not show a significant effect on YAP phosphorylation (FIG. 26B). These results indicate PKA rather than Epac mediates the effect of cAMP on YAP inhibition.

PKA C subunits form a complex with R subunits under basal state and the kinase activity is restricted; cAMP binding to the R subunits induces a conformational change and releases the C subunits and therefore, results in PKA kinase activation (FIG. 24) (Taylor et al. 2012). To study the involvement of PKA on YAP inactivation, we employed mutant PKA R subunits that interact with PKA C subunits in a manner unresponsive to cAMP. When mutant PKA R subunits (RIα and RIIα) were overexpressed, forskolin induced YAP phosphorylation was completely blocked (FIG. 26C). We also used shRNA to knockdown the PKA C subunits, and when PKA Ca expression was down regulated, the induction of YAP phosphorylation by forskolin or epinephrine was strongly compromised (FIG. 26D). Moreover, when cells were treated with a PKA inhibitor, KT5720 (FIG. 24), YAP phosphorylation was decreased, and the effect of forskolin and epinephrine was largely blocked by KT5720 (FIG. 26E). We also examined primary hepatocytes isolated from mice. Stimulation with glucagon, a ligand known to activate PKA, increased YAP phosphorylation (FIG. 26F). Similar to that of epinephrine, PKA inhibitor KT5720 abolished the effect of glucagon on YAP phosphorylation (FIG. 26F). Collectively, these data establish that PKA is the key mediator of cAMP in stimulating YAP phosphorylation.

cAMP Stimulates Lats Kinases to Induce YAP Phosphorylation.

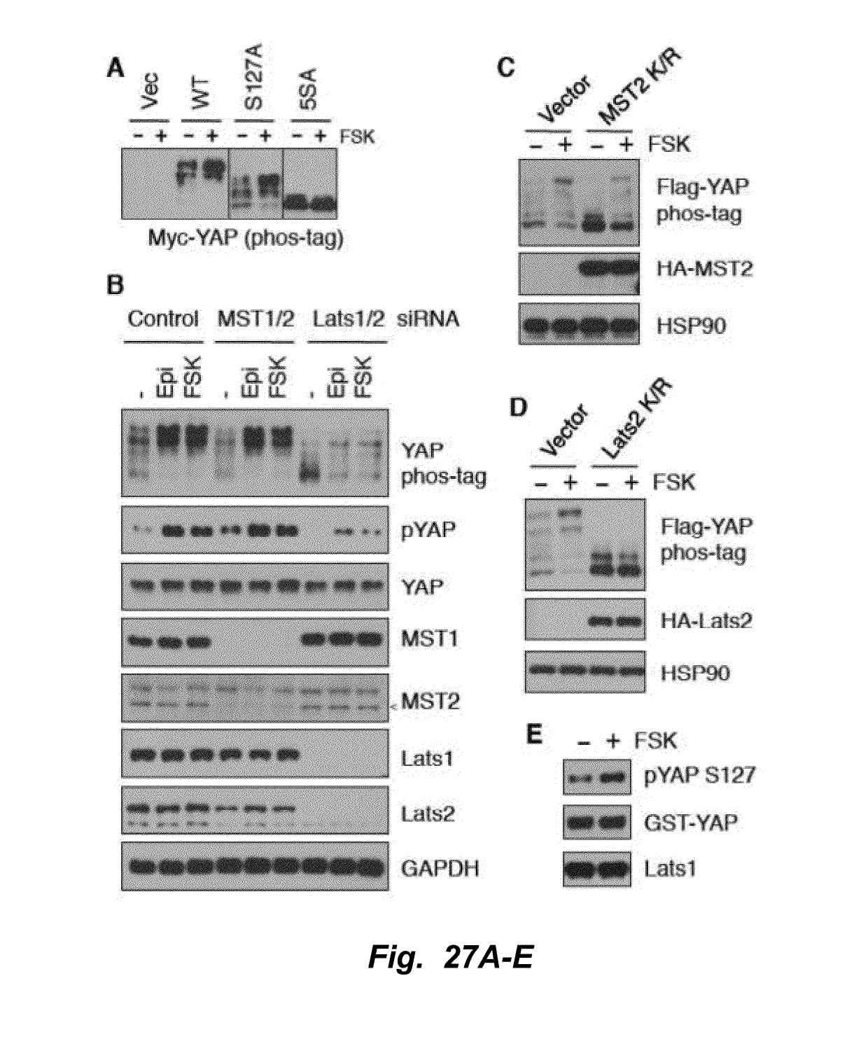

YAP is phosphorylated by the Lats1/2 kinases on five serine residues within the HXRXXS motifs, including S127, and can be phosphorylated on additional sites by other kinases (Zhao et al. 2010). To determine if cAMP regulates the Lats phosphorylation sites in YAP, a 5SA mutant YAP (with all five Lats targeting sites mutated to alanine) was transfected into cells and then treated with or without forskolin. Forskolin failed to induce a significant change in phosphorylation of the YAP-55A mutant, as assessed by phos-tag gel (FIG. 27A), suggesting that PKA likely acts through the Hippo-YAP pathway kinases to stimulate YAP phosphorylation. To test the function of MST kinases in cAMP induced YAP phosphorylation, MST1/2 expression was down regulated by siRNAs, and the phosphorylation status of YAP was relatively normal in response to forskolin treatment (FIG. 27B). Overexpression of MST2 K/R (lysine mutated to arginine), a kinase dead mutant, resulted in a lower basal YAP phosphorylation. However under this condition, forskolin was still capable to induce YAP phosphorylation (FIG. 27C), suggesting that MST may not be involved in cAMP response. In contrast, when Lats1/2 expression was down regulated by siRNAs, the basal YAP phosphorylation was lower, and notably forskolin induced YAP phosphorylation was significantly impaired (FIG. 27B). When Lats2 K/R, a kinase dead mutant, was overexpressed, the basal YAP phosphorylation was reduced, and importantly the effect of forskolin on YAP phosphorylation was abolished (FIG. 27D). These data indicate that Lats is involved in YAP phosphorylation in response to cAMP.

We next determined if cAMP could increase Lats kinase activity. Lats1 kinase was immunoprecipitated from cells treated with or without forskolin, and In vitro Lats kinase activity was measured using purified GST-YAP as a substrate. Our results indicate that Lats kinase activity is indeed induced by forskolin (FIG. 27E). Collectively, the above results reveal that Lats kinases are required for cAMP and PKA to induce YAP phosphorylation.

Rho GTPases are Required for PKA to Modulate YAP Phosphorylation.

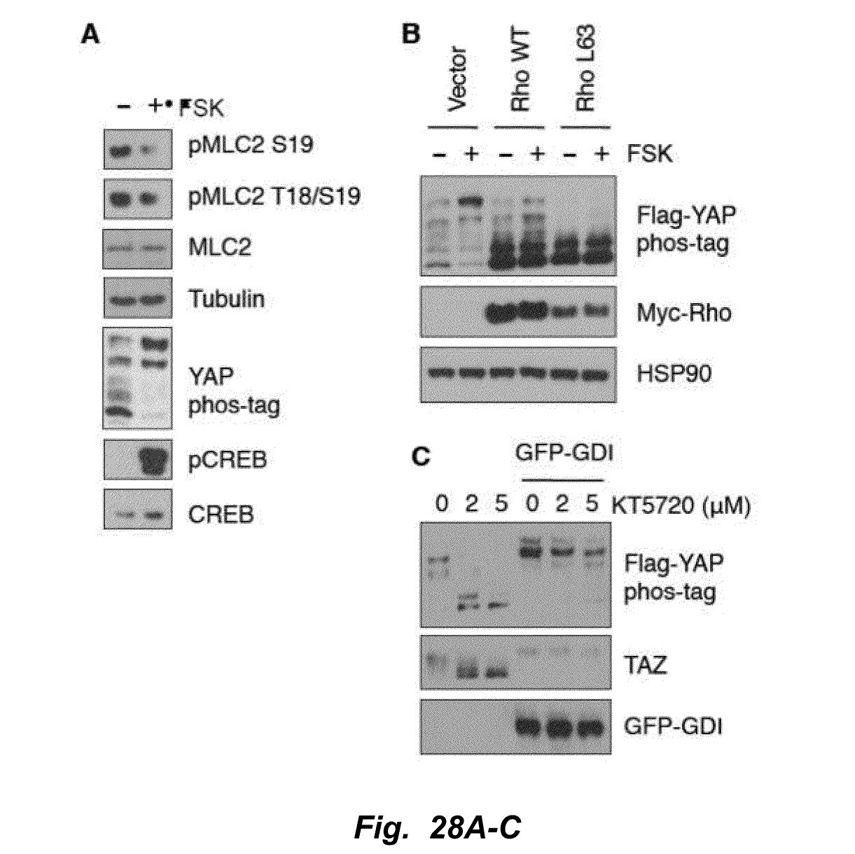

The response of YAP phosphorylation to cAMP is slower than that of CREB phosphorylation (FIG. 25C), suggesting that PKA may not directly phosphorylate a core component of the Hippo-YAP pathway. Consistently, we could not activate Lats1 in vitro using purified PKA (data not shown). Recently it has been reported that Rho GTPases can regulate the Hippo-YAP pathway and plays a major role from Ga12/13-coupled receptors to YAP phosphorylation (Dupont et al. 2011; Miller et al. 2012; Mo et al. 2012; Yu et al. 2012b; Zhao et al. 2012). Interestingly, PKA has been shown to modulate actin cytoskeleton by inhibition of RhoA, which is achieved by phosphorylation of RhoA, Rho GDP-dissociation inhibitor (RhoGDI), or Rho guanine nucleotide exchange factors (RhoGEF) (Qiao et al. 2008; Meiri et al. 2009; Tkachenko et al. 2011). PKA might induce Lats1/2 activity by repressing RhoA. Indeed, the phosphorylation of myosin light chain 2 (MLC2), a target of Rho-associated protein kinase (ROCK), was reduced when cells were treated with forskolin (FIG. 28A), indicating a decreased RhoA activity when PKA is activated. When cells were transfected with wild type or constitutively active RhoA, forskolin was unable to induce YAP phosphorylation (FIG. 28B). Complementarily, overexpression of RhoGDI, an inhibitor of Rho GTPases, the PKA inhibitor KT5720 was unable to induce YAP/TAZ dephosphorylation (FIG. 28C). Taken together, these observations suggest that RhoA is a major mediator for cAMP or PKA to regulate YAP phosphorylation, and we propose that PKA increases Lats1/2 activity and YAP phosphorylation by inhibiting Rho GTPases.

Hippo-YAP Pathway Activation is Required for cAMP- or PKA-Induced Adipogenesis.

PKA and cAMP play important roles in cell lineage specification during metazoan development (Lane and Kalderon 1993). For instance, PKA has been shown to promote adipogenesis (Rosen and MacDougald 2006), although molecular mechanisms underlying PKA regulated cell differentiation are not fully understood. Notably, TAZ displays activity opposite to PKA and can inhibit adipogenesis (Hong et al. 2005). As shown above, YAP and TAZ are negatively regulated by PKA, therefore the Hippo-YAP pathway might function downstream of PKA in regulating cell differentiation.

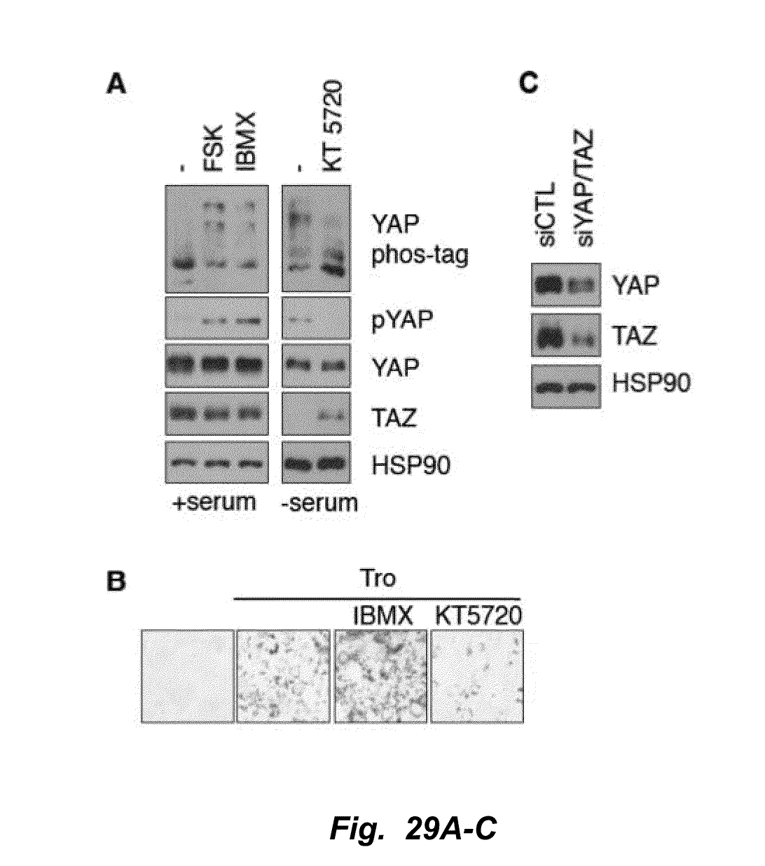

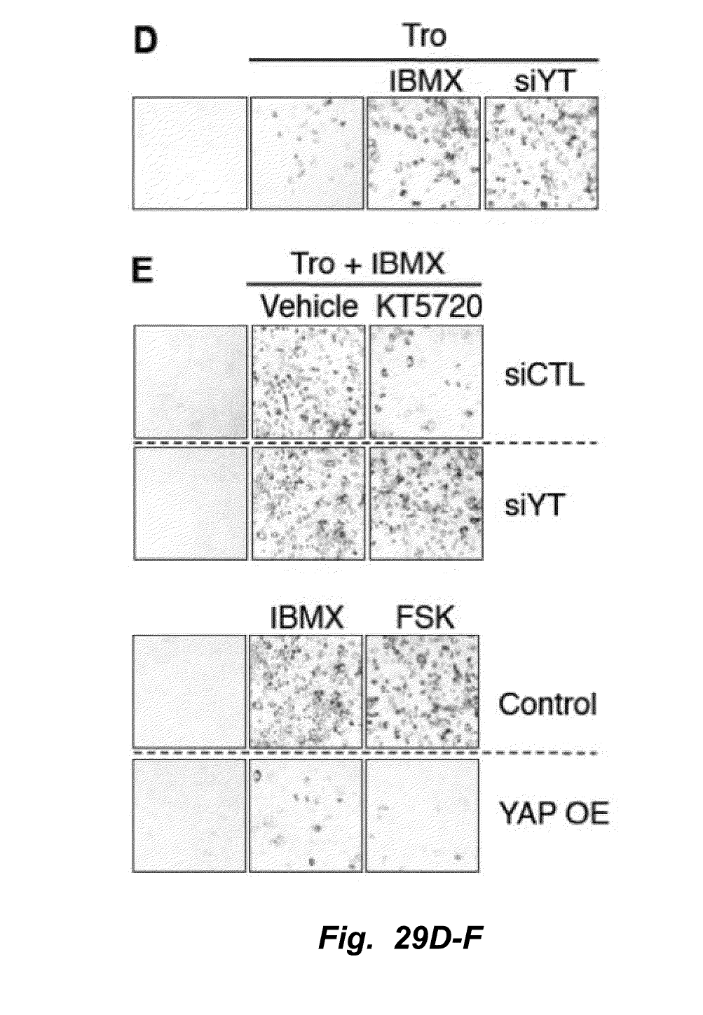

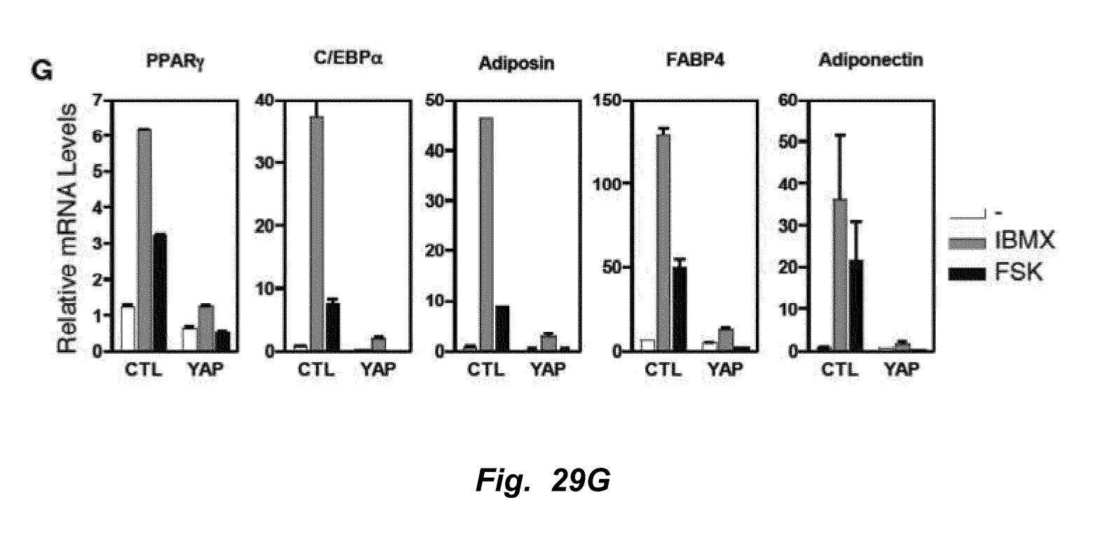

The effect of PKA or the Hippo-YAP pathway on adipocyte differentiation of murine fibroblast 3T3-L1 cells was examined. We found that YAP phosphorylation was repressed by KT5720 and induced by forskolin or IBMX in 3T3-L1 cells (FIG. 29A). TAZ protein level was increased by KT5720 and decreased by forskolin or IBMX (FIG. 29A), consistent with TAZ degradation upon phosphorylation (Liu et al. 2010). Adipogenesis was initiated by addition of insulin, dexamethasone and troglitazone (Tro), with or without PKA activator or inhibitor. The formation of lipid droplets was visualized by oil red staining. As expected, IBMX induced whereas KT5720 inhibited adipogenesis, indicating a stimulatory role of PKA in adipocyte differentiation (FIG. 29B). Next we examined the effect of YAP and TAZ on adipogenesis and found that knockdown of YAP and TAZ promoted adipogenesis, an effect similar to IBMX treatment (FIG. 29C,D). Interestingly, knockdown of YAP and TAZ also largely blocked the effect of PKA inhibitor KT5720 (FIG. 29E). On the other hand, YAP overexpression strongly inhibited the ability of IBMX or forskolin to induce adipogenesis (FIG. 29F). Furthermore the expression of multiple adipogenesis markers was abolished by YAP overexpression even in the presence of IBMX or forskolin (FIG. 29G). Therefore, YAP/TAZ activation is required for PKA inhibitor to suppress adipocyte differentiation whereas YAP/TAZ inhibition is crucial for IBMX to induce the differentiation program. Taken together, our data support a model that modulation of YAP and TAZ activity is required for PKA singling to regulate adipogenesis.

Inactivation of Yorki (Yki) by PKA in Drosophila.

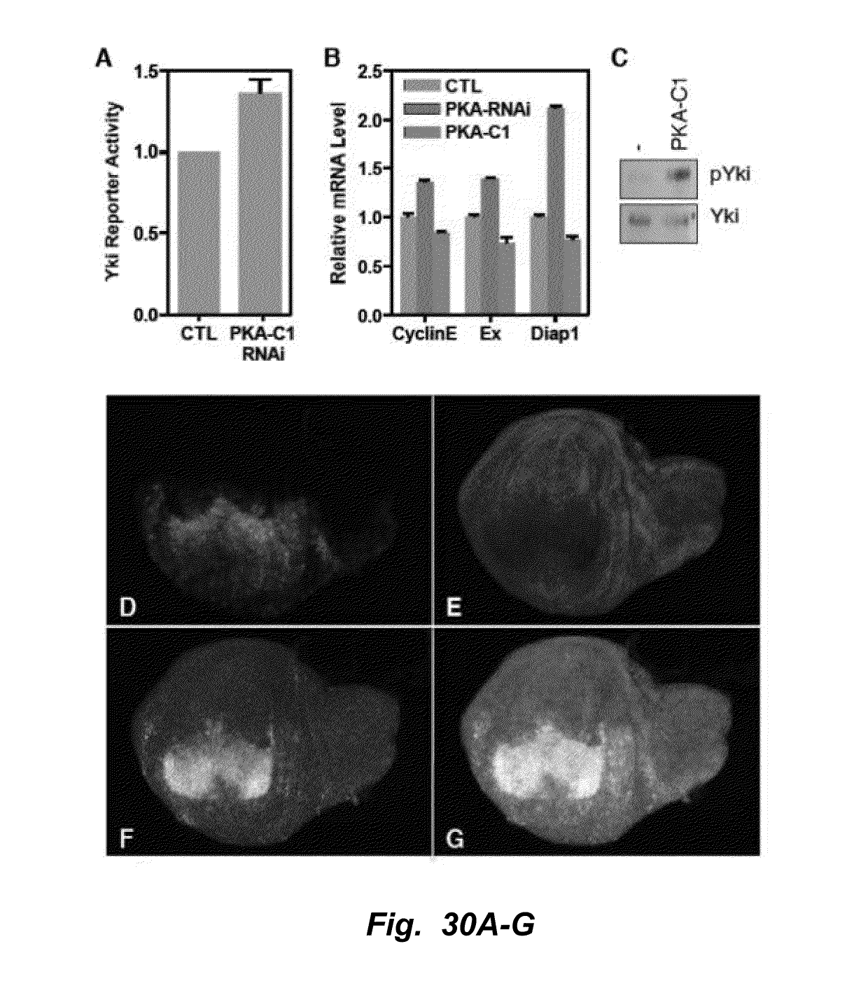

We next investigated whether the Drosophila YAP ortholog (Yki) is similarly regulated by PKA. In Drosophila S2R+ cells, when Drosophila PKA ortholog (PKA-C1) was knocked down by double stranded RNA (RNAi), the Yki transcription activity was significantly increased as assessed by luciferase assay (FIG. 30A). Therefore, the crosstalk between PKA and the Hippo-YAP pathway might be conserved in Drosophila. During Drosophila imaginal disc development, PKA-C1 has been shown to be a potent growth inhibitor and loss of PKA function leads to ectopic limb such as wing formation (Jiang and Struhl 1995; Lepage et al. 1995; Li et al. 1995; Pan and Rubin 1995). To test whether PKA-C1 regulates Hpo signaling for growth control, expression of several Yki target genes, including expanded (ex), Cyclin E (CycE) and Diap1 were determined at the transcript level. In larval wing discs, loss of PKA-C1 activity caused an increase of expression of ex, CycE and Diap1 (FIG. 30B). On the contrary, overexpression of PKA-C1 resulted in a moderate, yet significant, reduction of expression of these genes (FIG. 30B). Moreover, PKA-C1 overexpression induced Yki phosphorylation (FIG. 30C) and was sufficient to reduce the level of Diap1 protein and promote programmed cell death as revealed by increased caspase 3 staining (FIG. 30D-G). These results suggest that PKA inhibits Yki activity in developing tissues to restrict proliferation and promote apoptosis.

### Discussion

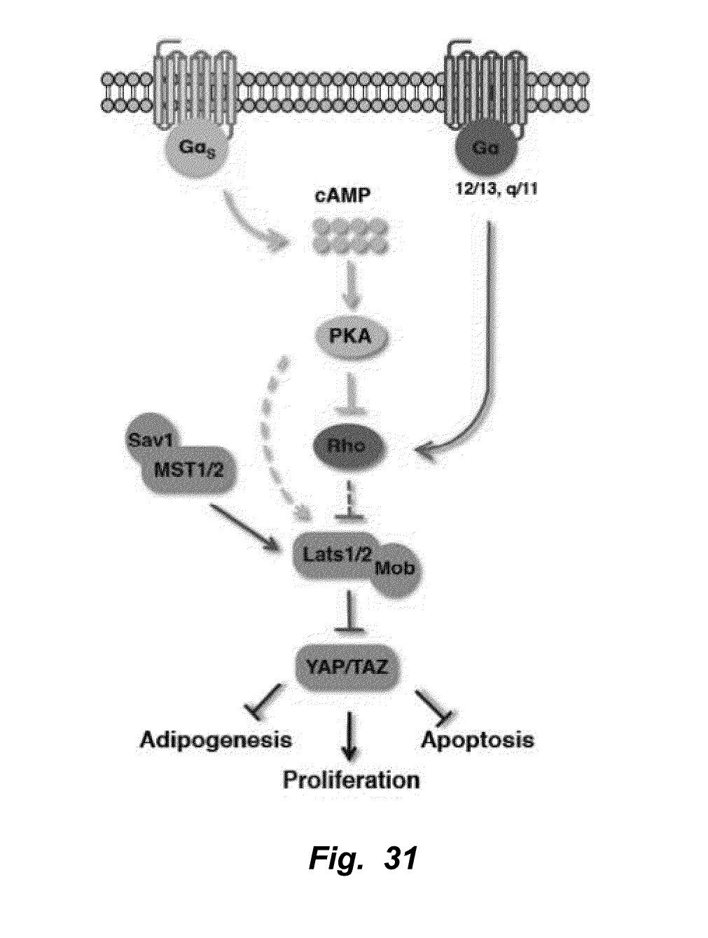

The signaling relay from cAMP to the Hippo-YAP pathway. In this report, we show that cAMP acts through PKA to stimulate Lats kinase activity and YAP phosphorylation, and the Rho GTPases likely mediate the effect of PKA to Hippo-YAP regulation (FIG. 31). Although the Hippo homolog MST1/2 may not be involved in YAP regulation in response to cAMP, we would still prefer to retain the name of Hippo given the fact that YAP/TAZ are the only known major functional output of the Hippo-YAP pathway. Our data establish Hippo-YAP as a physiologically relevant signaling branch downstream of PKA. The precise molecular mechanism connecting Rho to Lats kinase requires further investigation.

The kinase activity of endogenous Lats1 (FIG. 27E) and overexpressed Lats2 (data not shown) are increased upon PKA activation, and the effect of PKA activation on YAP phosphorylation is blocked by a kinase dead mutant Lats2, suggesting that the effect of PKA on YAP phosphorylation is mediated by Lats kinases. In contrast, MST1/2 are not required for PKA induced YAP phosphorylation, because the effect of forskolin and epinephrine on YAP phosphorylation is intact when both MST1 and MST2 are down-regulated by siRNA (FIG. 27B), and expression of kinase dead mutant MST2 does not block the effect of forskolin on YAP phosphorylation (FIG. 27C). Consistently, MST1 kinase activity and phosphorylation at the activation loop are not modulated by forskolin treatment (Yu et al. 2012b). However, phosphorylation of the hydrophobic motif of Lats1 is induced by cAMP signaling (Yu et al. 2012b), indicating that a kinase other than MST may phosphorylate the hydrophobic motif of Lats kinases upon PKA activation. Though unlikely, we cannot exclude the possibility that residual amount of MST kinase activity is sufficient to activate Lats in response to cAMP. Moreover, it is also possible that PKA may promote Lats phosphorylation by inhibiting a phosphatase.

PKA phosphorylates proteins containing RRXS/T consensus sequence and several components of the Hippo-YAP pathway with RRXS/T motif might be direct targets of PKA. Neurofibromin 2 (NF2, also known as merlin), a tumor suppressor and an upstream component of the Hippo-YAP pathway (McCartney et al. 2000; Hamaratoglu et al. 2006; Benhamouche et al. 2010; Zhang et al. 2010), has been shown as a direct target of PKA (Alfthan et al. 2004). Based on our data, NF2 is not critical for PKA to induce YAP phosphorylation, because the MDA-MB-231 cells have homozygous NF2 mutation (Dupont et al. 2011) while YAP phosphorylation is properly regulated by cAMP. PKA can also phosphorylate mouse Lats2 at 5171 and 5362 following forskolin treatment, with 5171 site conserved in Lats1 and warts (Drosophila Lats ortholog). However mutation of S171 or S362 of mouse Lats2 cannot block forskolin induced Lats2 activation (unpublished observations), indicating that Lats1/2 are unlikely to be direct targets of PKA responsible for cAMP-induced YAP phosphorylation. Our data are consistent with a model that Rho functions between PKA and Lats1/2 kinases (FIG. 31).

RhoA regulates the Hippo-YAP pathway by modulating actin cytoskeleton. Formation of actin filaments or generation of cellular tension results in YAP dephosphorylation, nuclear localization, and activation (Dupont et al. 2011; Fernandez et al. 2011; Sansores-Garcia et al. 2011; Wada et al. 2011; Mo et al. 2012; Yu et al. 2012b; Zhao et al. 2012). In addition to RhoA, other Rho family members, such as Rac and Cdc42, can also regulate the Hippo-YAP pathway kinases (Zhao et al. 2012). Therefore, the effect of PKA on the Hippo-YAP pathway may not be solely mediated by RhoA. Other Rho GTPases or their effectors may participate in the signaling pathway from PKA to Lats (FIG. 31). In support, PKA has been shown to phosphorylate PAK (Howe and Juliano 2000), which in principle can lead to rearrangements of actin cytoskeleton.

Gα_{12/13}- and Gαq/11-mediated signaling activate Rho GTPases and YAP (Yu et al. 2012b). The importance of Rho GTPases in PKA mediated YAP inactivation suggests that Gαₛ-mediated signals antagonize with Gα12/13- and Gαq/11-mediated signals on the activity of Rho GTPases, which in turn results in induction or repression of YAP phosphorylation. Therefore, differential regulations of Rho GTPases by numerous extracellular molecules will fine-tune the activity of the Hippo-YAP pathway and determine cellular responses such as cell proliferation, apoptosis, and differentiation (FIG. 31). This also provides a mechanism of signal integration when cells have to respond to a wide range of extracellular signals.

YAP/TAZ Inhibition Mediates Cellular Functions of PKA.

PKA is the first protein kinase purified and it is involved in a wide range of physiological regulations (Taylor et al. 2012). This report indicates that inhibition of YAP/TAZ contributes to the physiological function of cAMP or PKA. For example, cAMP can promote adipocyte differentiation and this process is dependent on inhibition of YAP and TAZ (FIG. 29). Besides adipocyte differentiation, PKA has been shown to induce neuronal differentiation and inhibit osteoblast differentiation (Ravni et al. 2006; Yang et al. 2008). YAP/TAZ plays a role in neurogenesis or osteogenesis in response to cAMP signals. Consistent with this notion, YAP or TAZ has been shown to promote osteogenesis and inhibit neuronal differentiation (Hong et al. 2005; Cao et al. 2008; Zhang et al. 2012) Interestingly, RhoA has similar functions as YAP/TAZ during various cell differentiation processes (McBeath et al. 2004). Therefore, inhibition of Rho GTPases and YAP/TAZ may serve as a common mechanism in PKA regulated cell differentiation.

PKA exerts growth inhibitory effect on most cell and tissue types. YAP and TAZ are putative oncoproteins and their activation stimulates cell proliferation and inhibits apoptosis. Therefore, PKA may inhibit cell growth by inactivating YAP/TAZ. This notion is supported by the functional analyses in Drosophila, in which PKA inhibits the expression of cyclin E and Diap1. Based on the data presented in this report, we propose that inhibition of YAP/TAZ plays a key role in mediating the growth inhibitory effect of PKA. YAP/TAZ activation, either by increased protein expression or reduced phosphorylation, is associated with a large number of human cancers (Chan et al. 2008; Steinhardt et al. 2008). Many pharmaceutical drugs directly target cellular cAMP levels. Elevation of cAMP by either phosphodiesterase inhibitors or adenylate cyclase activators may suppress tumor growth, particularly for those with high activity of YAP or TAZ.

### REFERENCES

Alfthan, K., Heiska, L., Gronholm, M., Renkema, G. H., and Carpen, O. 2004. Cyclic AMP-dependent protein kinase phosphorylates merlin at serine 518 independently of p21-activated kinase and promotes merlin-ezrin heterodimerization. J Biol Chem 279(18): 18559-18566.
Benhamouche, S., Curto, M., Saotome, I., Gladden, A. B., Liu, C. H., Giovannini, M., and McClatchey, A. I. 2010. Nf2/Merlin controls progenitor homeostasis and tumorigenesis in the liver. Genes Dev 24(16): 1718-1730.
Callus, B. A., Verhagen, A. M., and Vaux, D. L. 2006. Association of mammalian sterile twenty kinases, Mst1 and Mst2, with hSalvador via C-terminal coiled-coil domains, leads to its stabilization and phosphorylation. The FEBS journal 273(18): 4264-4276.
Cao, X., Pfaff, S. L., and Gage, F. H. 2008. YAP regulates neural progenitor cell number via the TEA domain transcription factor. Genes Dev 22(23): 3320-3334.
Chan, E. H., Nousiainen, M., Chalamalasetty, R. B., Schafer, A., Nigg, E. A., and Sillje, H. H. 2005. The Ste20-like kinase Mst2 activates the human large tumor suppressor kinase Lats1. Oncogene 24(12): 2076-2086.
Chan, S. W., Lim, C. J., Guo, K., Ng, C. P., Lee, I., Hunziker, W., Zeng, Q., and Hong, W. 2008. A role for TAZ in migration, invasion, and tumorigenesis of breast cancer cells. Cancer Res 68(8): 2592-2598.
Cho-Chung, Y. S. 1990. Role of cyclic AMP receptor proteins in growth, differentiation, and suppression of malignancy: new approaches to therapy. Cancer Res 50(22): 7093-7100.
Dong, J., Feldmann, G., Huang, J., Wu, S., Zhang, N., Comerford, S. A., Gayyed, M. F., Anders, R. A., Maitra, A., and Pan, D. 2007. Elucidation of a universal size-control mechanism in Drosophila and mammals. Cell 130(6): 1120-1133.
Dupont, S., Morsut, L., Aragona, M., Enzo, E., Giulitti, S., Cordenonsi, M., Zanconato, F., Le Digabel, J., Forcato, M., Bicciato, S. et al. 2011. Role of YAP/TAZ in mechanotransduction. Nature 474(7350): 179-183.
Fernandez, B. G., Gaspar, P., Bras-Pereira, C., Jezowska, B., Rebelo, S. R., and Janody, F. 2011. Actin-Capping Protein and the Hippo-YAP pathway regulate F-actin and tissue growth in Drosophila. Development 138(11): 2337-2346.
Gloerich, M. and Bos, J. L. 2010. Epac: defining a new mechanism for cAMP action. Annu Rev Pharmacol Toxicol 50: 355-375.
Goulev, Y., Fauny, J. D., Gonzalez-Marti, B., Flagiello, D., Silber, J., and Zider, A. 2008. SCALLOPED interacts with YORKIE, the nuclear effector of the hippo tumor-suppressor pathway in Drosophila. Current biology: CB 18(6): 435-441.
Hamaratoglu, F., Willecke, M., Kango-Singh, M., Nolo, R., Hyun, E., Tao, C., Jafar-Nejad, H., and Halder, G. 2006. The tumour-suppressor genes NF2/Merlin and Expanded act through Hippo signalling to regulate cell proliferation and apoptosis. Nature cell biology 8(1): 27-36.
Harvey, K. F., Pfleger, C. M., and Hariharan, I. K. 2003. The Drosophila Mst ortholog, hippo, restricts growth and cell proliferation and promotes apoptosis. Cell 114(4): 457-467.
Hong, J. H., Hwang, E. S., McManus, M. T., Amsterdam, A., Tian, Y., Kalmukova, R., Mueller, E., Benjamin, T., Spiegelman, B. M., Sharp, P. A. et al. 2005. TAZ, a transcriptional modulator of mesenchymal stem cell differentiation. Science 309(5737): 1074-1078.
Howe, A. K. and Juliano, R. L. 2000. Regulation of anchorage-dependent signal transduction by protein kinase A and p21-activated kinase. Nature cell biology 2(9): 593-600.
Huang, J., Wu, S., Barrera, J., Matthews, K., and Pan, D. 2005. The Hippo-YAP signaling pathway coordinately regulates cell proliferation and apoptosis by inactivating Yorkie, the Drosophila Homolog of YAP. Cell 122(3): 421-434.
Jia, J., Zhang, W., Wang, B., Trinko, R., and Jiang, J. 2003. The Drosophila Ste20 family kinase dMST functions as a tumor suppressor by restricting cell proliferation and promoting apoptosis. Genes Dev 17(20): 2514-2519.
Jiang, J. and Struhl, G. 1995. Protein kinase A and hedgehog signaling in Drosophila limb development. Cell 80(4): 563-572.
Justice, R. W., Zilian, O., Woods, D. F., Noll, M., and Bryant, P. J. 1995. The Drosophila tumor suppressor gene warts encodes a homolog of human myotonic dystrophy kinase and is required for the control of cell shape and proliferation. Genes Dev 9(5): 534-546.
Kanai, F., Marignani, P. A., Sarbassova, D., Yagi, R., Hall, R. A., Donowitz, M., Hisaminato, A., Fujiwara, T., Ito, Y., Cantley, L. C. et al. 2000. TAZ: a novel transcriptional co-activator regulated by interactions with 14-3-3 and PDZ domain proteins. EMBO J 19(24): 6778-6791.
Kango-Singh, M., Nolo, R., Tao, C., Verstreken, P., Hiesinger, P. R., Bellen, H. J., and Halder, G. 2002. Shar-pei mediates cell proliferation arrest during imaginal disc growth in Drosophila. Development 129(24): 5719-5730.
Lai, Z. C., Wei, X., Shimizu, T., Ramos, E., Rohrbaugh, M., Nikolaidis, N., Ho, L. L., and Li, Y. 2005. Control of cell proliferation and apoptosis by mob as tumor suppressor, mats. Cell 120(5): 675-685.
Lane, M. E. and Kalderon, D. 1993. Genetic investigation of cAMP-dependent protein kinase function in Drosophila development. Genes Dev 7(7A): 1229-1243.
Lei, Q. Y., Zhang, H., Zhao, B., Zha, Z. Y., Bai, F., Pei, X. H., Zhao, S., Xiong, Y., and Guan, K. L. 2008. TAZ promotes cell proliferation and epithelial-mesenchymal transition and is inhibited by the Hippo-YAP pathway. Molecular and cellular biology 28(7): 2426-2436.
Lepage, T., Cohen, S. M., Diaz-Benjumea, F. J., and Parkhurst, S. M. 1995. Signal transduction by cAMP-dependent protein kinase A in Drosophila limb patterning. Nature 373(6516): 711-715.
Li, W., Ohlmeyer, J. T., Lane, M. E., and Kalderon, D. 1995. Function of protein kinase A in hedgehog signal transduction and Drosophila imaginal disc development. Cell 80(4): 553-562.
Liu, C. Y., Zha, Z. Y., Zhou, X., Zhang, H., Huang, W., Zhao, D., Li, T., Chan, S. W., Lim, C. J., Hong, W. et al. 2010. The hippo tumor pathway promotes TAZ degradation by phosphorylating a phosphodegron and recruiting the SCF{beta}-TrCP E3 ligase. J Biol Chem 285(48): 37159-37169.
McBeath, R., Pirone, D. M., Nelson, C. M., Bhadriraju, K., and Chen, C. S. 2004. Cell shape, cytoskeletal tension, and RhoA regulate stem cell lineage commitment. Developmental cell 6(4): 483-495.
McCartney, B. M., Kulikauskas, R. M., LaJeunesse, D. R., and Fehon, R. G. 2000. The neurofibromatosis-2 homologue, Merlin, and the tumor suppressor expanded function together in Drosophila to regulate cell proliferation and differentiation. Development 127(6): 1315-1324.
Meiri, D., Greeve, M. A., Brunet, A., Finan, D., Wells, C. D., LaRose, J., and Rottapel, R. 2009. Modulation of Rho guanine exchange factor Lfc activity by protein kinase A-mediated phosphorylation. Molecular and cellular biology 29(21): 5963-5973.
Miller, E., Yang, J., Deran, M., Wu, C., Su, A. I., Bonamy, G. M., Liu, J., Peters, E. C., and Wu, X. 2012. Identification of Serum-Derived Sphingosine-1-Phosphate as a Small Molecule Regulator of YAP. Chem Biol 19(8): 955-962.
Mo, J. S., Yu, F. X., Gong, R., Brown, J. H., and Guan, K. L. 2012. Regulation of the Hippo-YAP pathway by protease activated receptor PAR. Genes Dev 29(19): doi:10.1101/gad.197582.197112.
Oh, H. and Irvine, K. D. 2008. In vivo regulation of Yorkie phosphorylation and localization. Development 135(6): 1081-1088.
Pan, D. and Rubin, G. M. 1995. cAMP-dependent protein kinase and hedgehog act antagonistically in regulating decapentaplegic transcription in Drosophila imaginal discs. Cell 80(4): 543-552.
Pantalacci, S., Tapon, N., and Leopold, P. 2003. The Salvador partner Hippo promotes apoptosis and cell-cycle exit in Drosophila. Nature cell biology 5(10): 921-927.
Qiao, J., Holian, O., Lee, B. S., Huang, F., Zhang, J., and Lum, H. 2008. Phosphorylation of GTP dissociation inhibitor by PKA negatively regulates RhoA. Am J Physiol Cell Physiol 295(5): C1161-1168.
Ravni, A., Bourgault, S., Lebon, A., Chan, P., Galas, L., Fournier, A., Vaudry, H., Gonzalez, B., Eiden, L. E., and Vaudry, D. 2006. The neurotrophic effects of PACAP in PC12 cells: control by multiple transduction pathways. J Neurochem 98(2): 321-329.
Ren, F., Zhang, L., and Jiang, J. 2010. Hippo signaling regulates Yorkie nuclear localization and activity through 14-3-3 dependent and independent mechanisms. Developmental biology 337(2): 303-312.
Rosen, E. D. and MacDougald, O. A. 2006. Adipocyte differentiation from the inside out. Nat Rev Mol Cell Biol 7(12): 885-896.
Sansores-Garcia, L., Bossuyt, W., Wada, K., Yonemura, S., Tao, C., Sasaki, H., and Halder, G. 2011. Modulating F-actin organization induces organ growth by affecting the Hippo-YAP pathway. EMBO J 30(12): 2325-2335.
Sassone-Corsi, P. 2012. The cyclic AMP pathway. Cold Spring Harb Perspect Biol 4(12).
Steinhardt, A. A., Gayyed, M. F., Klein, A. P., Dong, J., Maitra, A., Pan, D., Montgomery, E. A., and Anders, R. A. 2008. Expression of Yes-associated protein in common solid tumors. Human pathology 39(11): 1582-1589.
Stork, P. J. and Schmitt, J. M. 2002. Crosstalk between cAMP and MAP kinase signaling in the regulation of cell proliferation. Trends Cell Biol 12(6): 258-266.
Tapon, N., Harvey, K. F., Bell, D. W., Wahrer, D. C., Schiripo, T. A., Haber, D. A., and Hariharan, I. K. 2002. salvador Promotes both cell cycle exit and apoptosis in Drosophila and is mutated in human cancer cell lines. Cell 110(4): 467-478.
Taylor, S. S., Ilouz, R., Zhang, P., and Kornev, A. P. 2012. Assembly of allosteric macromolecular switches: lessons from PKA. Nat Rev Mol Cell Biol 13(10): 646-658.
Tkachenko, E., Sabouri-Ghomi, M., Pertz, O., Kim, C., Gutierrez, E., Machacek, M., Groisman, A., Danuser, G., and Ginsberg, M. H. 2011. Protein kinase A governs a RhoA-RhoGDI protrusion-retraction pacemaker in migrating cells. Nature cell biology 13(6): 660-667.
Udan, R. S., Kango-Singh, M., Nolo, R., Tao, C., and Halder, G. 2003. Hippo promotes proliferation arrest and apoptosis in the Salvador/Warts pathway. Nature cell biology 5(10): 914-920.
Wada, K., Itoga, K., Okano, T., Yonemura, S., and Sasaki, H. 2011. Hippo-YAP pathway regulation by cell morphology and stress fibers. Development 138(18): 3907-3914.
Wu, S., Huang, J., Dong, J., and Pan, D. 2003. hippo encodes a Ste-20 family protein kinase that restricts cell proliferation and promotes apoptosis in conjunction with salvador and warts. Cell 114(4): 445-456.
Wu, S., Liu, Y., Zheng, Y., Dong, J., and Pan, D. 2008. The TEAD/TEF family protein Scalloped mediates transcriptional output of the Hippo growth-regulatory pathway. Developmental cell 14(3): 388-398.
Xu, T., Wang, W., Zhang, S., Stewart, R. A., and Yu, W. 1995. Identifying tumor suppressors in genetic mosaics: the Drosophila lats gene encodes a putative protein kinase. Development 121(4): 1053-1063.
Yang, D. C., Tsay, H. J., Lin, S. Y., Chiou, S. H., Li, M. J., Chang, T. J., and Hung, S. C. 2008. cAMP/PKA regulates osteogenesis, adipogenesis and ratio of RANKL/OPG mRNA expression in mesenchymal stem cells by suppressing leptin. PLoS One 3(2): e1540.
Yu, F. X. and Guan, K. L. 2013. The Hippo-YAP pathway: regulators and regulations. Genes Dev 27(4): 355-371.
Yu, F. X., Mo, J. S., and Guan, K. L. 2012a. Upstream regulators of the Hippo-YAP pathway. Cell Cycle 11(22): Epub.
Yu, F. X., Zhao, B., Panupinthu, N., Jewell, J. L., Lian, I., Wang, L. H., Zhao, J., Yuan, H., Tumaneng, K., Li, H. et al. 2012b. Regulation of the Hippo-YAP Pathway by G-Protein-Coupled Receptor Signaling. Cell 150(4): 780-791.
Zhang, H., Deo, M., Thompson, R. C., Uhler, M. D., and Turner, D. L. 2012. Negative regulation of Yap during neuronal differentiation. Developmental biology 361(1): 103-115.
Zhang, L., Ren, F., Zhang, Q., Chen, Y., Wang, B., and Jiang, J. 2008. The TEAD/TEF family of transcription factor Scalloped mediates Hippo signaling in organ size control. Developmental cell 14(3): 377-387.
Zhang, N., Bai, H., David, K. K., Dong, J., Zheng, Y., Cai, J., Giovannini, M., Liu, P., Anders, R. A., and Pan, D. 2010. The Merlin/NF2 tumor suppressor functions through the YAP oncoprotein to regulate tissue homeostasis in mammals. Developmental cell 19(1): 27-38.
Zhao, B., Li, L., Tumaneng, K., Wang, C. Y., and Guan, K. L. 2010. A coordinated phosphorylation by Lats and CK1 regulates YAP stability through SCF(beta-TRCP). Genes Dev 24(1): 72-85.
Zhao, B., Li, L., Wang, L., Wang, C. Y., Yu, J., and Guan, K. L. 2012. Cell detachment activates the Hippo-YAP pathway via cytoskeleton reorganization to induce anoikis. Genes Dev 26(1): 54-68.
Zhao, B., Wei, X., Li, W., Udan, R. S., Yang, Q., Kim, J., Xie, J., Ikenoue, T., Yu, J., Li, L. et al. 2007. Inactivation of YAP oncoprotein by the Hippo-YAP pathway is involved in cell contact inhibition and tissue growth control. Genes Dev 21(21): 2747-2761.
Zhao, B., Ye, X., Yu, J., Li, L., Li, W., Li, S., Yu, J., Lin, J. D., Wang, C. Y., Chinnaiyan, A. M. et al. 2008. TEAD mediates YAP-dependent gene induction and growth control. Genes Dev 22(14): 1962-1971.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of preventing, reducing, delaying or inhibiting the proliferation, growth, migration and/or metastasis of a cancer cell or tumor comprising contacting the cancer cell or tumor with an effective amount of an inhibitor of transcriptional coactivator with PDZ binding motif (TAZ)/Yes-associated protein (YAP) transcription co-activator, wherein the inhibitor of TAZ/YAP comprises a 9H-Fluoren-9-one, oxime pharmacophore of Formula II:

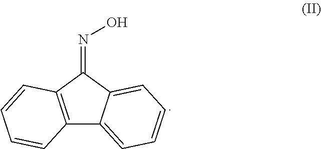 [SMILES: ON=C1c2ccccc2-c2ccccc21]

2. The method of claim 1, wherein the proliferation and/or growth of the cancer cell is mediated by unphosphorylated TAZ/YAP.

3. A method of preventing, reducing, delaying or inhibiting the proliferation, growth, migration and/or metastasis of a cancer cell or tumor mediated by activation of transcriptional coactivator with PDZ binding motif (TAZ)/Yes-associated protein (YAP) transcription co-activator in a subject in need thereof, comprising administering to the subject an effective amount of an inhibitor of TAZ/YAP, wherein the inhibitor of TAZ/YAP comprises a 9H-Fluoren-9-one, oxime pharmacophore of Formula II:

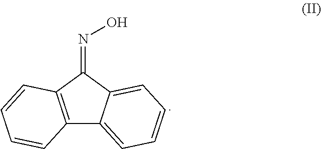 [SMILES: ON=C1c2ccccc2-c2ccccc21]

4. A method of preventing, reducing and/or inhibiting the dephosphorylation of transcriptional coactivator with PDZ binding motif (TAZ)/Yes-associated protein (YAP) transcription co-activator and/or promoting and/or increasing the ubiquitination and/or degradation of TAZ/YAP in a cancer cell or tumor, comprising contacting the cancer cell or tumor with an effective amount of an inhibitor of TAZ/YAP, wherein the inhibitor of TAZ/YAP comprises a 9H-Fluoren-9-one, oxime pharmacophore of Formula II:

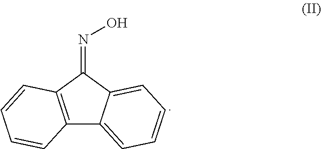 [SMILES: ON=C1c2ccccc2-c2ccccc21]

5. The method of any one of claims 1 to 4, wherein the inhibitor of TAZ/YAP comprises an oxime derivative of 9-fluorenone bearing two piperidinylsulfonyl groups.

6. The method of any one of claims 1 to 5, wherein the inhibitor of TAZ/YAP comprises 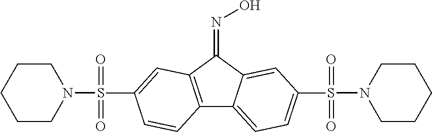 [SMILES: O=S(=O)(c1ccc2c(c1)C(=NO)c1cc(S(=O)(=O)N3CCCCC3)ccc1-2)N1CCCCC1]
2,7-bis(piperidin-1-yl-sulfonyl)-9H-fluoren-9-one oxime.

7. The method of any one of claims 1 to 6, wherein the inhibitor of TAZ/YAP prevents, reduces or inhibits TAZ/YAP protein levels and/or the dephosphorylation and/or nuclear translocation and/or localization of TAZ/YAP.

8. The method of any one of claims 1 to 7, wherein the cancer cell or tumor is in vitro.

9. The method of any one of claims 1 to 7, wherein the cancer cell or tumor is in vivo.

10. The method of any one of claims 1 to 9, wherein the cancer cell or tumor is in a human subject.

11. The method of any one of claims 1 to 10, wherein the inhibitor of TAZ/YAP is administered orally, intravenously, inhalationally, transdermally, subcutaneously or intramuscularly.

12. A method of preventing, reducing, delaying or inhibiting the proliferation, growth, migration and/or metastasis of a cancer cell or tumor comprising contacting the cancer cell or tumor with an effective amount of an inhibitor of a Gα-protein selected from the group consisting of G12, G13, Gq, G11, Gi and Go or an antagonist of a G-protein-coupled receptor (GPCR) coupled to a Gα-protein selected from the group consisting of G12, G13, Gq, G11, Gi and Go.

13. A method of preventing, reducing, delaying or inhibiting the proliferation, growth, migration and/or metastasis of a cancer or tumor mediated by activation of TAZ/YAP in a subject in need thereof, comprising administering to the subject an effective amount of an inhibitor of a Gα-protein selected from the group consisting of G12, G13, Gq, G11, Gi and Go or an inhibitor of a G-protein-coupled receptor (GPCR) coupled to a Gα-protein selected from the group consisting of G12, G13, Gq, G11, Gi and Go.

14. The method of any one of claims 12 to 13, wherein the G-protein-coupled receptor (GPCR) is selected from the group consisting of lysophosphatidic acid receptor 1-5 (LPAR1-5), sphingosine 1-phosphate receptors, coagulation factor II (thrombin) receptors, estrogen receptor 1 (GPR30), frizzled homolog D4, bombesin-like receptor 3, adrenergic receptor alpha 1B, a lysophosphatidic acid receptor, purinergic receptor 1, purinergic receptor type A, 5-hydroxytryptamine receptor 4, muscarinic acetylcholine receptor M1, adenosine receptor A1A, angiotensin II receptor, free fatty acid receptor 1, platelet-activating factor receptor, thromboxane a2 receptor, complement component 3a receptor 1, glutamate receptor metabotropic 2, opioid receptor delta 1, secretin receptor, thyroid stimulating hormone receptor, gastrin-releasing peptide receptor, melanocortin receptor 1, somatostatin receptor 1 and prostaglandin E receptor 2.

15. A method of preventing, reducing, delaying or inhibiting the proliferation, growth, migration and/or metastasis of a cancer cell or tumor comprising contacting the cancer cell or tumor with an effective amount of an activator of a Gs Gα-protein or an agonist of a G-protein-coupled receptor (GPCR) coupled to a Gs Gα-protein.

16. A method of preventing, reducing, delaying or inhibiting the proliferation, growth, migration and/or metastasis of a cancer or tumor mediated by activation of TAZ/YAP in a subject in need thereof, comprising administering to the subject an effective amount of an activator of a Gs Gα-protein or an agonist of a G-protein-coupled receptor (GPCR) coupled to a Gs Gα-protein.

17. The method of any one of claim 15 or 16, wherein the Gs G-protein-coupled receptor (GPCR) is selected from the group consisting of endothelin receptor type A, chemokine (C—X—C motif) receptor 4, CXCR2, adrenergic receptor beta 2, dopamine receptor D1, glucagon receptor, and epinephrine receptor.

18. A method of preventing, reducing, delaying or inhibiting the proliferation, growth, migration and/or metastasis of a cancer cell or tumor comprising contacting the cancer cell or tumor with an effective amount of an activator of adenylyl cyclase (AC) and/or an inhibitor of phosphodiesterase (PDE).

19. A method of preventing, reducing, delaying or inhibiting the proliferation, growth, migration and/or metastasis of a cancer or tumor mediated by activation of TAZ/YAP in a subject in need thereof, comprising administering to the subject an effective amount of an activator of adenylyl cyclase (AC) and/or an inhibitor of phosphodiesterase (PDE).

20. The method of any one of claims 18 to 19, wherein the inhibitor of phosphodiesterase is an inhibitor of PDE4.

21. The method of claim 20, wherein the inhibitor of PDE4 is selected from the group consisting of rolipram, roflumilast, cilomilast, ariflo, HT0712, ibudilast, mesembrine, pentoxifylline, piclamilast, and combinations thereof.

22. The method of claim 20, wherein the inhibitor of phosphodiesterase is an inhibitor of PDE5.

23. The method of any one of claims 1 to 22, wherein the cancer is selected from the group consisting of melanoma, uveal melanoma, breast cancer, liver cancer, hepatocellular carcinoma, lung adenocarcinoma, glioma, colon cancer, colorectal cancer, mesothelioma, gastric cancer, medulloblastoma, ovarian cancer, esophageal cancer, esophageal squamous cell carcinoma, sarcoma, Ewing sarcoma, head and neck cancer, prostate cancer, and meningioma.

24. A method of preventing, reducing and/or inhibiting signaling through the HIPPO-YAP/TAZ cell signaling pathway and/or preventing, reducing and/or inhibiting YAP/TAZ activation and/or dephosphorylation in a cell, comprising contacting the cell with a ligand selected from the group consisting of lysophosphatidic acid, sphingosine 1-phosphate (S1P) and thrombin.

25. A method of preventing, reducing and/or inhibiting signaling through the HIPPO-YAP/TAZ cell signaling pathway and/or preventing, reducing and/or inhibiting YAP/TAZ activation and/or dephosphorylation in a cell, comprising contacting the cell with an antagonist of a G-protein-coupled receptor (GPCR) selected from the group consisting of lysophosphatidic acid receptor 1-5 (LPAR1-5), sphingosine 1-phosphate receptors, coagulation factor II (thrombin) receptors, estrogen receptor 1 (GPR30), frizzled homolog D4, bombesin-like receptor 3, adrenergic receptor alpha 1B, a lysophosphatidic acid receptor, purinergic receptor 1, purinergic receptor type A, 5-hydroxytryptamine receptor 4, muscarinic acetylcholine receptor M1, adenosine receptor A1A, angiotensin II receptor, free fatty acid receptor 1, platelet-activating factor receptor, thromboxane a2 receptor, complement component 3a receptor 1, glutamate receptor metabotropic 2, opioid receptor delta 1, secretin receptor, thyroid stimulating hormone receptor, gastrin-releasing peptide receptor, melanocortin receptor 1, somatostatin receptor 1 and prostaglandin E receptor 2.

26. A method of preventing, reducing and/or inhibiting signaling through the HIPPO-YAP/TAZ cell signaling pathway and/or preventing, reducing and/or inhibiting YAP/TAZ activation and/or dephosphorylation in a cell, comprising contacting the cell with an agonist of a G-protein-coupled receptor (GPCR) selected from the group consisting of endothelin receptor type A, chemokine (C—X—C motif) receptor 4, CXCR2, adrenergic receptor beta 2, dopamine receptor D1, glucagon receptor, and epinephrine receptor.

27. A method of preventing, reducing and/or inhibiting signaling through the HIPPO-YAP/TAZ cell signaling pathway and/or preventing, reducing and/or inhibiting YAP/TAZ activation and/or dephosphorylation in a cell, comprising contacting the cell with an actin disrupting agent.

28. A method of preventing, reducing and/or inhibiting signaling through the HIPPO-YAP/TAZ cell signaling pathway and/or preventing, reducing and/or inhibiting YAP/TAZ activation and/or dephosphorylation in a cell, comprising contacting the cell with an activator of adenylyl cyclase (AC) and/or an inhibitor of phosphodiesterase (PDE).

29. The method of claim 28, wherein the inhibitor of phosphodiesterase is an inhibitor of PDE4.

30. The method of claim 29, wherein the inhibitor of PDE4 is selected from the group consisting of rolipram, roflumilast, cilomilast, ariflo, HT0712, ibudilast, mesembrine, pentoxifylline, piclamilast, and combinations thereof.

31. The method of claim 28, wherein the inhibitor of phosphodiesterase is an inhibitor of PDE5.

32. A method of preventing, reducing and/or inhibiting signaling through the HIPPO-YAP/TAZ cell signaling pathway and/or preventing, reducing and/or inhibiting YAP/TAZ activation and/or dephosphorylation in a cell, comprising contacting the cell with a ligand selected from the group consisting of glucagon, epinephrine and a dopamine receptor agonist.

33. The method of any one of claims 24 to 31, wherein the cell is a cancer cell.

34. A method of activating, promoting and/or increasing signaling through the HIPPO-YAP/TAZ cell signaling pathway and/or activating, promoting and/or increasing signaling through the HIPPO-YAP/TAZ cell signaling pathway in a cell, comprising contacting the cell with a ligand selected from the group consisting of glucagon, epinephrine and a dopamine receptor antagonist.

35. A method of activating, promoting and/or increasing signaling through the HIPPO-YAP/TAZ cell signaling pathway and/or activating, promoting and/or increasing signaling through the HIPPO-YAP/TAZ cell signaling pathway in a cell, comprising contacting the cell with an agonist of a G-protein-coupled receptor (GPCR) selected from the group consisting of lysophosphatidic acid receptor 1-5 (LPAR1-5), sphingosine 1-phosphate receptors, coagulation factor II (thrombin) receptors, estrogen receptor 1 (GPR30), frizzled homolog D4, bombesin-like receptor 3, adrenergic receptor alpha 1B, a lysophosphatidic acid receptor, purinergic receptor 1, purinergic receptor type A, 5-hydroxytryptamine receptor 4, muscarinic acetylcholine receptor M1, adenosine receptor A1A, angiotensin II receptor, free fatty acid receptor 1, platelet-activating factor receptor, thromboxane a2 receptor, complement component 3a receptor 1, glutamate receptor metabotropic 2, opioid receptor delta 1, secretin receptor, thyroid stimulating hormone receptor, gastrin-releasing peptide receptor, melanocortin receptor 1, somatostatin receptor 1 and prostaglandin E receptor 2.

36. A method of activating, promoting and/or increasing signaling through the HIPPO-YAP/TAZ cell signaling pathway and/or activating, promoting and/or increasing signaling through the HIPPO-YAP/TAZ cell signaling pathway in a cell, comprising contacting the cell with an antagonist of a G-protein-coupled receptor (GPCR) selected from the group consisting of endothelin receptor type A, chemokine (C—X—C motif) receptor 4, CXCR2, adrenergic receptor beta 2, dopamine receptor D1, glucagon receptor, and epinephrine receptor.

37. The method of any one of claims 24 to 36, wherein the cell is in vivo.

38. The method of any one of claims 24 to 36, wherein the cell is in vitro.

39. A method of reducing or inhibiting the proliferation, growth, invasiveness and/or migration of a cell, comprising contacting the cell with an effective amount of an inhibitor of TAZ/YAP, wherein the inhibitor of TAZ/YAP comprises a 9H-Fluoren-9-one, oxime pharmacophore of Formula II:

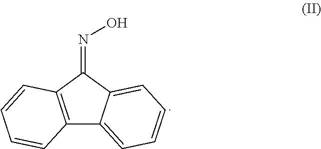 [SMILES: ON=C1c2ccccc2-c2ccccc21]

40. A method of preventing, reducing and/or inhibiting the dephosphorylation of transcriptional coactivator with PDZ binding motif (TAZ)/Yes-associated protein (YAP) transcription co-activator and/or promoting and/or increasing the ubiquitination and/or degradation of TAZ/YAP in a cell, comprising contacting the cell with an effective amount of an inhibitor of TAZ/YAP, wherein the inhibitor of TAZ/YAP comprises a 9H-Fluoren-9-one, oxime pharmacophore of Formula II:

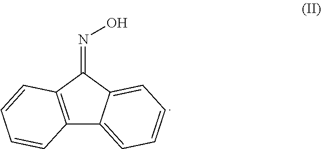 [SMILES: ON=C1c2ccccc2-c2ccccc21]

41. The method of any one of claims 39 to 40, wherein the inhibitor of TAZ/YAP comprises an oxime derivative of 9-fluorenone bearing two piperidinylsulfonyl groups.

42. The method of any one of claims 39 to 41, wherein the inhibitor of TAZ/YAP comprises 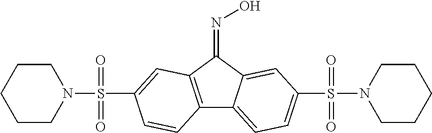 [SMILES: O=S(=O)(c1ccc2c(c1)C(=NO)c1cc(S(=O)(=O)N3CCCCC3)ccc1-2)N1CCCCC1]
2,7-bis(piperidin-1-yl-sulfonyl)-9H-fluoren-9-one oxime.

43. The method of any one of claims 39 to 42, wherein the inhibitor of TAZ/YAP prevents, reduces or inhibits YAP/TAZ protein levels and/or the dephosphorylation and/or nuclear localization of TAZ/YAP.

44. The method of any one of claims 39 to 43, wherein the cell is in vivo.

45. The method of any one of claims 39 to 43, wherein the cell is in vitro.

46. The method of any one of claims 39 to 45, wherein the cell is in a human subject.

47. The method of any one of claims 39 to 46, wherein the inhibitor of TAZ/YAP is administered orally, intravenously, inhalationally, transdermally, subcutaneously or intramuscularly.
